SEQUENCE LISTING (46 sequences)

SEQ ID NO: 1 - DNA, 885 bp, Homo sapiens
  source 1..885: mol_type = other DNA
atggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacagaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaa

SEQ ID NO: 2 - DNA, 885 bp, synthetic construct
  misc_feature 1..885: note = CoSMN2
  source 1..885: mol_type = other DNA
atggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaa

SEQ ID NO: 3 - DNA, 885 bp, synthetic construct
  misc_feature 1..885: note = CoSMN3
  source 1..885: mol_type = other DNA
atggcaatgtcttccggtggatctgggggcggagtcccagaacaggaagactccgtattgttcagaagagggacagggcaatctgacgactccgatatttgggatgacacggccctcattaaggcatatgacaaggcagtggccagtttcaaacatgctctgaaaaatggcgacatttgcgaaacatccgggaagccgaagacaacaccaaaacgtaagcccgcaaagaaaaataagtctcagaagaagaacactgctgccagtcttcagcagtggaaagtgggagacaaatgttccgcaatctggtctgaggacgggtgtatttaccccgccacgatcgcttctatcgactttaagagagaaacttgtgtggtggtgtataccggctacgggaaccgcgaagagcaaaacctgtccgacctgctgtcccccatctgcgaggtggccaacaatatcgagcagaacgctcaggagaacgaaaacgagagtcaagtgtctacagatgaaagcgagaattctcggtctccaggaaataaatctgacaatatcaaaccgaaaagcgccccctggaattcctttttgccccctccacctccaatgcctgggcctagactgggtcctgggaagcccggcctcaagttcaatggaccgcctcccccacccccccctcccccgccccatctcctcagttgttggctcccccccttcccatccgggcccccaatcattcctccgccgcccccaatttgtccagacagcctggacgacgctgacgctttagggtccatgctcatctcttggtatatgagtggctatcataccggttattacatgggtttcaggcagaaccagaaggagggccggtgttcgcatagcctaaattag

SEQ ID NO: 4 - DNA, 885 bp, synthetic construct
  misc_feature 1..885: note = CoSMN4
  source 1..885: mol_type = other DNA
atggccatgagcagcggcggatctggcggcggcgtgcctgagcaggaggatagcgtgctgttccggagaggcaccggacagtccgatgattctgacatctgggacgacaccgccctgatcaaggcctacgacaaggccgtggcttccttcaagcacgctctgaaaaatggcgacatctgtgaaaccagcggcaagcccaagacaacccctaagagaaagcctgccaagaaaaacaagtctcaaaagaagaacaccgccgcttctctgcagcagtggaaggtgggcgataagtgcagcgctatctggtccgaggacggctgtatctaccctgccaccatcgccagcatcgacttcaagcgggaaacatgcgtggtggtctacacaggctacggcaaccgggaagaacaaaacctgagcgatctgctcagccctatctgcgaggtggccaacaacatcgagcagaatgcccaggaaaacgagaatgaaagccaggtttccaccgacgagtctgagaacagccgcagccccggaaacaagagcgacaacatcaagcctaagtccgccccttggaacagctttctgcctcctccgccccccatgcctggccctagactgggccccggcaaacctggactgaaattcaacggcccccccccacccccccctcctcctccacctcacctgctgagctgctggctgcctcccttcccttctgggcctccaattatccctccacctcctcctatctgccccgacagcctggacgacgccgacgccctgggcagcatgctgatcagctggtacatgagcggctaccacacaggctactacatgggctttagacagaaccagaaagagggcagatgcagccattctctgaattga

SEQ ID NO: 5 - DNA, 885 bp, synthetic construct
  misc_feature 1..885: note = CoSMN5
  source 1..885: mol_type = other DNA
atggccatgtcctccggtgggtcaggtgggggggtccctgaacaggaagacagtgtcttgtttcgacgcgggacgggccaaagtgacgattctgatatttgggacgataccgcactcattaaggcttacgataaggctgtggcgagcttcaagcacgctttgaagaacggagatatttgcgagacgtctggcaagccgaaaacaaccccgaagcgcaagcccgctaagaagaataagtcccagaaaaaaaacacagctgccagtcttcagcagtggaaggtgggagataaatgctctgccatatggtctgaggatgggtgtatttacccggcaacaatcgccagcatcgacttcaaacgagaaacgtgcgtggtcgtttataccgggtatgggaacagggaggaacagaacctcagcgatcttctttctcccatttgtgaggtggccaacaacatcgaacaaaatgcgcaggaaaatgagaatgagagtcaagtgtctaccgatgaaagtgaaaattctcgcagcccgggaaacaagtctgacaatatcaagcccaagtctgcaccatggaacagtttcttgccgcctccgccaccgatgccggggcctagattggggccgggcaaaccgggacttaagttcaacggaccccctccaccgccccctcctccgccaccgcacttgttgtcatgttggctccctccatttcccagtggtcctcctatcataccacctcctccgccgatttgtcccgatagccttgatgacgctgatgctttgggatccatgcttatctcttggtatatgtcaggataccataccgggtattacatgggattcagacagaaccagaaagaggggcggtgcagccactccctcaactga

SEQ ID NO: 6 - DNA, 885 bp, synthetic construct
  misc_feature 1..885: note = CoSMN6
  source 1..885: mol_type = other DNA
atggccatgtctagtggcggatctggtggcggagtgcccgagcaagaagatagcgtcctgttcagaagaggcaccggccagagcgacgacagcgacatctgggatgatacagccctgatcaaggcctacgacaaggccgtggccagctttaagcacgccctgaagaacggcgatatctgcgagacaagcggcaagcccaagaccacacctaagagaaagcccgccaagaagaacaagagccagaagaagaataccgccgccagcctgcagcagtggaaagtgggcgataagtgcagcgccatttggagcgaggacggctgtatctaccctgccacaatcgccagcatcgacttcaagcgggaaacctgcgtggtggtgtacacaggctacggcaacagagaggaacagaacctgagcgacctgctgagcccaatttgcgaggtggccaacaacatcgagcagaacgcccaagagaacgagaacgagtcccaggtgtccaccgacgagagcgagaatagcagaagccccggcaacaagagcgacaacatcaagcctaagagcgccccttggaacagcttcctgcctcctcctccaccaatgcctggacctagactcggacctggaaagcccggcctgaagttcaatggacctccaccaccgccaccacctccgcctccacatcttctgtcttgttggctgcctccatttcctagcggccctccaatcatcccgccacctccacctatctgccccgacagtctggatgatgctgatgccctgggctccatgctgatctcttggtacatgagcggctaccacaccggctactacatgggcttcagacagaaccagaaagagggccgttgcagccacagcctgaactga

SEQ ID NO: 7 - DNA, 885 bp, synthetic construct
  misc_feature 1..885: note = CoSMN7
  source 1..885: mol_type = other DNA
atggctatgagcagcggaggaagcggaggcggagtgcccgaacaagaggattccgtgctgtttagaaggggaaccggacagtccgatgatagcgacatctgggacgacaccgctctgatcaaggcctacgacaaagccgtggcctccttcaagcatgctctgaagaacggcgacatctgcgaaaccagcggcaagcccaaaacaacccccaagaggaagcccgccaagaagaacaagtcccagaagaagaacaccgccgcttctctgcagcagtggaaggtgggcgacaagtgtagcgccatctggtccgaggatggctgcatctatcccgccaccattgcctccatcgacttcaagagggagacatgcgtggtggtgtacaccggctacggcaatagagaggaacagaatctgtccgatctgctgagccctatctgcgaggtggccaacaacattgagcagaacgcccaagagaacgagaacgaaagccaagtgtccaccgacgagagcgaaaacagcagaagccccggcaacaagagcgacaacatcaagcccaagtccgccccttggaactcctttctgccccctcctccccctatgcccggccctagactgggacccggcaagcccggactgaagttcaacggacccccccctcccccccccccccctcctcctcatctgctgagctgttggctccctcctttccccagcggccctcctatcatccctcctcctcctcccatctgccccgattctctggatgatgccgatgctctgggcagcatgctgatcagctggtacatgagcggctatcacaccggctactacatgggcttcagacagaaccagaaggagggcagatgctcccactctctgaactga

SEQ ID NO: 8 - DNA, 885 bp, synthetic construct
  misc_feature 1..885: note = CoSMN8
  source 1..885: mol_type = other DNA
atggctatgtcctctggagggtccggcggaggcgtgccggagcaggaagactccgttctgttccgccgtggcacagggcagtcagacgattccgacatctgggatgacactgccctcatcaaggcctacgataaggctgtcgctagctttaagcatgccctgaagaacggagacatctgtgagacatccggtaagcccaagaccacgcccaagcggaagcccgccaaaaagaacaagagtcagaaaaagaacaccgctgcatctcttcaacagtggaaagtgggcgacaagtgctccgctatctggagcgaggacgggtgcatctatccagctaccatcgcatctatcgactttaagcgcgagacctgcgtagtggtctatactggttacggcaatagggaggaacagaacttgtctgatcttctgtccccgatctgcgaagtggccaataacattgaacaaaacgcccaggagaacgagaacgagagccaggtctccacggacgagagcgagaacagtcgctcccctggcaacaagtccgacaacatcaaaccgaagtctgcgccctggaactcattcctcccacctccgcccccaatgcctggtccccgcctgggccccggcaagccggggctcaagtttaatggacctcccccgcccccacccccaccccctccccacctgctctcttgctggttgcccccattcccctccgggccacctatcattcccccaccgcctccaatctgcccagactctctggacgatgccgatgccctgggctctatgctgatctcctggtatatgtccggctatcataccggctattacatgggattccgccagaaccaaaaggagggccgctgtagccactccctgaactga

SEQ ID NO: 9 - DNA, 885 bp, synthetic construct; identical to SEQ ID NO: 1
  misc_feature 1..885: note = CoSMN9
  source 1..885: mol_type = other DNA

SEQ ID NO: 10 - DNA, 885 bp, synthetic construct
  misc_feature 1..885: note = CoSMN10
  source 1..885: mol_type = other DNA
atggccatgtcgtccggaggctccggtggtggggtgcctgagcaagaggattccgtcctcttcaggcgcgggaccgggcaaagtgatgactcggatatctgggacgatactgccttaatcaaagcctacgataaagccgttgctagctttaaacacgctctgaagaatggcgacatctgcgagacaagtgggaagcccaagaccaccccaaagcggaagcctgcaaaaaagaataagtcacagaaaaaaaacacagccgcgagcctgcagcagtggaaggtcggcgataagtgttctgccatttggtctgaggacggttgtatctaccctgcgaccatcgcctcaattgactttaaacgggagacctgcgtggtagtctacacaggctacggcaaccgcgaggaacagaacctgtcagatttgcttagcccgatctgtgaggtggccaataacattgaacagaatgcacaggaaaatgaaaatgaatcccaggtgagcaccgacgagtctgaaaattctcggtcccccggtaataaatcggacaatataaaaccaaaaagcgccccgtggaactctttcctcccacctcctccccccatgccagggccacgccttggcccaggtaagccgggcctcaagttcaacggccccccaccccccccccctccccctcccccgcacttactgagctgttggctacccccatttccatcaggcccacccatcattccgccaccccctcccatctgtcctgattcactagatgacgcggatgccctcggctctatgctaatctcgtggtatatgagcggctatcacacaggttactacatgggcttccggcaaaatcagaaagagggccgctgctcgcactctctgaactaa

SEQ ID NO: 11 - DNA, 885 bp, synthetic construct
  misc_feature 1..885: note = CoSMN11
  source 1..885: mol_type = other DNA
atggccatgagcagcggcggcagcggcggcggcgtgcctgagcaggaggacagcgtgctgttcaggaggggcaccggccagagcgacgacagcgacatctgggacgacaccgccctgatcaaggcctacgacaaggccgtggccagcttcaagcacgccctgaagaacggcgacatctgcgagaccagcggcaagcctaagaccacccctaagaggaagcctgccaagaagaacaagagccagaagaagaacaccgccgccagcctgcagcagtggaaggtgggcgacaagtgcagcgccatctggagcgaggacggctgcatctaccctgccaccatcgccagcatcgacttcaagagggagacctgcgtggtggtgtacaccggctacggcaacagggaggagcagaacctgagcgacctgctgagccctatctgcgaggtggccaacaacatcgagcagaacgcccaggagaacgagaacgagagccaggtgagcaccgacgagagcgagaacagcaggagccctggcaacaagagcgacaacatcaagcctaagagcgccccttggaacagcttcctgcctcctcctcctcctatgcctggccctaggctgggccctggcaagcctggcctgaagttcaacggccctcctcctcctcctcctcctcctcctcctcacctgctgagctgctggctgcctcctttccctagcggccctcctatcatccctcctcctcctcctatctgccctgacagcctggacgacgccgacgccctgggcagcatgctgatcagctggtacatgagcggctaccacaccggctactacatgggcttcaggcagaaccagaaggagggcaggtgcagccacagcctgaactga

SEQ ID NO: 12 - DNA, 885 bp, synthetic construct
  misc_feature 1..885: note = CoSMN12
  source 1..885: mol_type = other DNA
atggctatgtccagcggtggatctgggggcggcgttcccgagcaggaggactccgtgctgttccgcaggggtactggccagagtgatgactctgacatatgggatgacaccgccctcattaaggcttacgacaaagcagtggccagtttcaagcacgctctgaaaaacggtgacatttgtgagacttctggcaaacctaaaaccactcctaaaagaaaacccgctaagaagaacaaatcacaaaagaagaatacagccgcctccttacagcagtggaaggtgggcgacaagtgctcagctatctggtccgaggacgggtgcatttaccctgccaccatcgcttcaatcgatttcaaaagggagacctgcgtggttgtgtatactgggtacggaaatcgagaagaacagaatctgtccgatctgctctcccccatatgcgaggtcgcaaacaacatagaacagaatgcacaggagaacgaaaatgagtcacaggtttcaactgatgaaagtgagaatagtaggtcccctggtaataaaagcgacaacataaagcccaaaagcgcaccatggaatagcttcctgccaccccccccaccaatgccagggccaaggctggggccaggcaagccgggtctaaagtttaatggtccgcccccaccgccccccccacccccaccccacttgctgtcatgttggcttccgccattcccttccggtccacctatcattcctccacccccaccaatttgcccagacagcttagacgacgcagacgccttgggctcgatgctgatcagctggtatatgtcgggataccatacaggctactatatgggtttccgtcagaaccaaaaggaaggccggtgttcccattccctgaattga

SEQ ID NO: 13 - DNA, 885 bp, synthetic construct
  misc_feature 1..885: note = CoSMN13
  source 1..885: mol_type = other DNA
atggccatgtcttcaggaggatcaggtgggggcgtccctgaacaggaggactctgtgctcttccggcgaggtacgggccaaagcgatgacagcgacatttgggatgacaccgcgctcatcaaggcctatgataaagctgttgcatccttcaagcacgctcttaaaaatggtgacatttgtgaaacttccgggaaacccaaaaccacacccaagaggaagcccgccaagaagaataaatctcagaagaaaaacactgctgcctccctgcagcagtggaaggtgggggacaagtgcagcgctatatggtctgaagatggctgcatctatccagcaaccattgcgtccattgacttcaaaagagagacctgtgtggtggtatacacaggatatggcaaccgggaggagcaaaacctgagcgacctgctatcacccatttgtgaggtcgccaacaacatcgagcagaatgcccaggaaaacgagaatgaaagccaagtttccacagatgaaagtgagaactccaggagtccagggaacaagtcagacaatataaaaccaaaaagtgcaccttggaatagctttctccctccaccaccacccatgcctgggccccgtttgggtcctggcaagcctggcctgaagtttaatggacctcctcccccaccaccaccaccgccccctcatttattgagctgctggctgcccccctttcctagtggaccgcccatcatccccccgccacctccaatctgccctgattctctggatgatgctgatgcattaggcagcatgctgatatcctggtacatgagtggctaccacactggttactatatggggttccgccagaaccagaaagaaggcagatgttcgcattcacttaattga

SEQ ID NO: 14 - DNA, 885 bp, synthetic construct
  misc_feature 1..885: note = CoSMN14
  source 1..885: mol_type = other DNA
atggccatgagctcagggggttctggaggcggtgtaccagaacaagaagactctgttctcttcaggagaggaacagggcaatctgatgacagcgacatctgggacgacactgctttaataaaggcgtatgacaaggctgtcgcttccttcaagcatgcacttaaaaatggagacatttgtgagactagtgggaaacctaagacaactccaaagaggaagcctgccaagaagaacaaaagccagaaaaaaaacacagcagcatccttgcagcagtggaaagtgggtgataaatgttcagcaatatggagcgaagatggctgcatctacccagccaccattgccagcattgattttaaaagagagacctgtgtggtcgtttacaccggctatggcaaccgggaggagcaaaacctgagtgacctgttgtctccgatctgtgaagtggccaacaacattgagcagaatgctcaggagaatgaaaatgagtcccaggtgtccaccgatgaatctgagaactcccgttcacccggaaataaatcggacaacatcaagcccaagagtgcgccctggaattccttccttcctccgccgcctcccatgcctgggcctcgattaggccctgggaagccagggctcaagttcaatggcccacccccaccaccacctccaccgcccccccacctactgtcgtgctggctgcctccttttccttctggtccccccattatccctcctcccccacccatctgcccagacagtctggatgatgctgatgccctgggaagcatgctgatctcctggtacatgagtggatatcacacgggttactacatgggctttcggcagaaccagaaggagggccgctgcagtcatagcctcaactga

SEQ ID NO: 15 - DNA, 885 bp, synthetic construct
  misc_feature 1..885: note = CoSMN15
  source 1..885: mol_type = other DNA
atggccatgagctccgggggaagtggagggggagttcctgagcaagaagacagcgtcctctttaggaggggtacaggccaatccgatgattcagatatttgggacgacactgctctgattaaagcatacgacaaagcggtggcaagtttcaagcatgcattgaaaaatggtgatatatgcgagacttcaggcaagcctaaaacgacacccaagagaaagccagctaagaaaaacaagagccaaaagaagaacacagccgccagcctccagcagtggaaggtcggtgataaatgttcagctatatggtcagaggacgggtgtatttatccagcaaccatcgccagcatcgacttcaaacgggagacctgtgtagtggtttacaccggatacggcaaccgggaagagcagaacctgtccgaccttctgtctccaatctgtgaggtggctaacaatatcgagcagaatgcccaggaaaatgagaatgaatcccaggtgtcgacggatgagtcggaaaactctcgaagtcccgggaataagtccgataatatcaaaccaaaaagtgccccctggaactctttcctcccacctcctccacctatgcccgggccccgcctgggccccgggaagcctggattgaagtttaacggcccccctccgccacccccgcccccaccgcctcatctgctaagttgctggctgcctccgtttccgtctggcccaccgattatcccccctccgccacccatttgcccagacagccttgacgatgcggacgccttaggttccatgttaatcagctggtatatgtccggctatcacaccggctactacatgggattcagacagaatcagaaggaaggccgctgcagccactctctgaactga

SEQ ID NO: 16 - DNA, 885 bp, synthetic construct
  misc_feature 1..885: note = CoSMN16
  source 1..885: mol_type = other DNA
atggcaatgagtagtggtggtagtggtggtggagtgcctgagcaggaagattccgtgctgttccggagaggcacaggccagtctgacgactctgatatctgggatgacaccgctctgatcaaggcctacgacaaggccgtggccagctttaagcacgccctgaagaatggcgacatttgtgaaacctctggcaaacccaagaccacccctaagcggaagcctgccaagaagaacaagtcccagaagaaaaacaccgccgcttctctgcagcaatggaaggtgggcgacaagtgctctgccatctggtccgaggacggctgcatctaccctgctaccatcgcctctatcgacttcaagagagagacatgtgtggtcgtgtacaccggctacggcaaccgggaggaacagaacctgtccgacctgctctctcccatctgcgaggtggctaacaacatcgagcaaaacgcccaggaaaacgagaacgagtcccaggtgtccaccgacgaatccgagaattcccgctctcctggcaataagtccgacaacatcaagcctaaaagcgctccttggaactccttcctgcctccacctcctccaatgcctggtcctagactgggccccggcaagcccggcctgaagttcaacggccctcctccacctcccccgcccccaccccctcacctgctgtcctgctggctgcctcctttcccttctggcccccccatcatccctccacctcctcctatctgccctgatagcctggacgatgccgacgctctgggatctatgctgatctcctggtatatgtccggataccacaccggctactacatgggcttcagacagaaccagaaagagggcagatgcagccattccctgaactga

SEQ ID NO: 17 - DNA, 885 bp, synthetic construct
  misc_feature 1..885: note = CoSMN17
  source 1..885: mol_type = other DNA
atggcaatgagttccggggggtcaggtggaggtgtgccagaacaagaagatagtgtgctcttccgtcggggcacaggccaatcagatgatagtgacatctgggatgacaccgcacttatcaaagcatatgataaagcagtagcttctttcaagcatgctctaaaaaatggagacatatgtgaaacgtcagggaaaccaaaaacaactcctaaaaggaagcctgcgaagaaaaacaagtctcagaagaagaacactgcagccagtttgcagcagtggaaagtgggagacaagtgcagtgctatttggtctgaggatggctgcatttaccctgctaccatagcatccattgattttaagagagagacttgtgtcgttgtctacacaggttatggcaacagagaagagcagaacctctcagacttacttagccccatttgtgaggttgccaacaatattgaacagaatgctcaagagaatgaaaatgagagtcaggtatctacagatgaatctgagaacagcaggagccctggaaataaatcagacaatataaagcccaaaagcgccccctggaattcttttttgcctcctccgcccccaatgccaggtccacgactgggcccaggaaaacctgggctaaagtttaatgggcctcctccacccccccctccgccaccgcctcatctgctgtcctgctggcttccaccatttccaagtggacctcccatcattcctcccccaccaccaatctgtccagattcgttagacgatgcagatgccctgggatctatgctgatatcctggtacatgtctggataccacacagggtattatatgggtttcagacaaaaccagaaagaaggacgctgctcacacagcttgaactga

SEQ ID NO: 18 - DNA, 885 bp, synthetic construct
  misc_feature 1..885: note = CoSMN18
  source 1..885: mol_type = other DNA
atggccatgagctccggtggtagtggcggaggcgtacctgaacaagaagactctgttttgtttcggaggggcaccggccaaagcgatgattcagatatatgggacgacaccgcgttaatcaaggcctacgataaagctgtggcatctttcaaacatgcattgaagaatggagacatctgcgagaccagcggaaaacccaagacaacgccaaagcgcaaacctgccaagaagaataagagccagaagaaaaatactgctgcctcactgcagcagtggaaggtgggggataaatgtagcgccatatggagcgaggatggctgcatctatccagctacaattgcatccattgacttcaagagggaaacttgcgtggtggtctacaccggatatggtaacagagaggaacagaatctctctgaccttctgtccccaatttgcgaggtcgccaacaacatagagcagaatgcccaggagaacgaaaatgaatcacaagtttcgacagatgaaagtgagaacagccgatccccggggaacaaatctgacaacatcaaaccaaagagtgcgccctggaattcatttctgcccccaccccctcccatgcctggaccacgtctaggtcctgggaaacctggccttaagttcaatggcccccctcctccacccccacctccaccgcctcacctgctgtcttgttggctgccgcccttccccagtgggccgcccatcattcctccgccccccccaatctgtccagacagcctggatgacgctgatgcactaggctccatgctcatttcctggtatatgtctgggtatcacactggatactacatggggtttcggcagaaccagaaagagggcagatgttcccattctctcaactga

SEQ ID NO: 19 - DNA, 885 bp, synthetic construct
  misc_feature 1..885: note = CoSMN19
  source 1..885: mol_type = other DNA
atggccatgtcttcgggcggctcgggtggcggtgtccccgaacaggaggactccgtgctgttccggcgcgggactggacagagcgatgacagtgacatatgggatgacaccgccctgatcaaggcgtacgacaaagccgtagcttctttcaagcacgccctgaagaatggggacatctgcgagacaagcggcaagcccaaaaccactcctaagcgcaagcctgccaagaaaaacaagtcccagaagaagaacacggcagccagcttgcaacagtggaaggtgggtgacaagtgttctgctatctggtctgaagacggctgtatttatccagcgaccatcgcatccattgactttaagagagagacctgcgtggtggtgtacaccggctacggcaaccgcgaggagcagaacctgtccgacctgctgtcgcccatctgcgaggtggccaacaacattgagcagaatgcgcaggagaatgagaacgagtcccaggtgtccaccgacgagagcgagaactcgcgtagcccaggcaacaagtccgacaacatcaaacccaagtcagctccgtggaactccttcctgcctcctccaccgccaatgcctggaccccgcctgggtccgggcaagcccggtctcaaatttaacggccctccacccccccccccgcctccccctccccacctgctttcatgctggcttccgcccttcccgtctgggccgcccatcatcccgcctccgccccccatctgcccagattccttggatgacgcggatgctctgggatctatgttaatcagttggtacatgagcggctaccacacgggctattacatgggcttccgccagaaccagaaggagggcaggtgtagccactccctcaactga

SEQ ID NO: 20 - DNA, 885 bp, synthetic construct
  misc_feature 1..885: note = CoSMN20
  source 1..885: mol_type = other DNA
atggccatgtcgtcagggggctcaggaggtggagttcctgagcaggaggatagtgtcctgttccgaaggggcaccggacaaagtgatgactctgacatttgggatgacaccgcgttgataaaggcatatgacaaagcagtggctagcttcaaacatgctcttaaaaatggtgacatctgtgaaacttctgggaagccaaaaacgacacccaaaaggaagccggccaagaagaataaatctcaaaagaaaaacactgcagcgtccctgcagcagtggaaggttggagacaagtgttcagccatctggagcgaagatggctgcatctatcctgccaccatcgcctccattgactttaaacgggagacctgtgtagtggtctacactggctatggtaatagagaggagcaaaatctttcagacctcctctctcccatttgtgaggtggccaacaacatcgagcagaatgcccaggaaaatgagaatgaaagccaggtgagtacagatgaaagtgaaaactcccgctctcccggcaacaagagtgacaacattaagcccaagtcagctccctggaactcctttttaccaccacctccgcccatgcctgggcctcggctaggcccaggtaaacccggtctgaagtttaatgggcctccacctccacccccgcctcccccaccccacctgctcagctgctggctgccccctttcccttcgggaccgcccataattccacctccaccaccgatctgccccgacagcttagatgatgctgatgcactggggagcatgctgatctcctggtacatgagcggctaccacacaggatactacatgggcttcagacagaaccagaaagagggccgctgctcccattctttgaactga

SEQ ID NO: 21 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 22 - DNA, 382 bp, synthetic construct
  misc_feature 1..382: note = CMV IE enhancer
  source 1..382: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 23 - DNA, 282 bp, synthetic construct
  misc_feature 1..282: note = CB promoter
  source 1..282: mol_type = other DNA
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 24 - DNA, 133 bp, synthetic construct
  misc_feature 1..133: note = PCI intron
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 25 - DNA, 127 bp, synthetic construct
  misc_feature 1..127: note = RGB polyA signal
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 26 - DNA, 105 bp, synthetic construct
  misc_feature 1..105: note = 5' ITR105
  source 1..105: mol_type = other DNA
ctgcgcgctcgctcgctcactgaggccgcccgggcaaagcccgggcgtcgggcgacctttggtcgcccggcctcagtgagcgagcgagcgcgcagagagggagtg

SEQ ID NO: 27 - DNA, 130 bp, synthetic construct
  misc_feature 1..130: note = 3' ITR130
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 28 - DNA, 511 bp, synthetic construct
  misc_feature 1..511: note = hPGK promoter
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 29 - DNA, 302 bp, synthetic construct
  misc_feature 1..302: note = chimeric intron CBA+rabbit globin
  source 1..302: mol_type = other DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattt

SEQ ID NO: 30 - DNA, 232 bp, synthetic construct
  misc_feature 1..232: note = bGH polyA signal
  source 1..232: mol_type = other DNA
ctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 31 - DNA, 469 bp, synthetic construct
  misc_feature 1..469: note = hSyn promoter
  source 1..469: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 32 - protein, 736 aa, Adeno-associated virus9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 621 aa, Adeno-associated virus 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 34 - DNA, 2211 bp, Adeno-associated virus9
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 35 - DNA, 1866 bp, Adeno-associated virus 2
  source 1..1866: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 36 - DNA, 720 bp, synthetic construct
  misc_feature 1..720: note = GFP coding sequence
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 37 - DNA, 9331 bp, synthetic construct
  misc_feature 1..9331: note = the transgene plasmid comprising CoSMN2
  source 1..9331: mol_type = other DNA
ttaattaacaattgacattattgaagcatttatcagggttattgtctcagactgcgcgctcgctcgctcactgaggccgcccgggcaaagcccgggcgtcgggcgacctttggtcgcccggcctcagtgagcgagcgagcgcgcagagagggagtgtgtagttaatgattaacccgccatgctacttatctaccagggtaatggggatcctctagaactatagctagtcgacataacgcgtcaattgctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggaagcttgtaagtatcaaggttacaagacaggtttaaggagaccaatagaaactgggcttgtcgagacagagaagactcttgcgtttctgataggcacctattggtcttactgacatccactttgcctttctctccacagggcctcgaggacggggtgaactacgcctgaggatccgccaccatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggtttcagacaaaatcaaaaagaaggaaggtgctcacattccttaaattaatgtacaggtacctcgatggatttccaaggttaattcattggaattgaaaattaacagagatctagagctgaattcctgcagccagggggatcagcctgatctttttccctctgccaaaaattatggggacatcatgaagccccttgagcatctgacttctggctaataaaggaaatttattttcattgcaatagtgtgttggaattttttgtgtctctcactcgcggaccgagcggccgcgcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccctgcctgcaggcatttaattaacctaattcggcagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 38 - DNA, 20 bp, synthetic construct
  misc_feature 1..20: note = Forward primer for ddPCR
  source 1..20: mol_type = other DNA
cccacttggcagtacatcaa

SEQ ID NO: 39 - DNA, 22 bp, synthetic construct
  misc_feature 1..22: note = Reverse primer for ddPCR
  source 1..22: mol_type = other DNA
gccaagtaggaaagtcccataa

SEQ ID NO: 40 - DNA, 24 bp, synthetic construct
  misc_feature 1..24: note = Probe for ddPCR
  source 1..24: mol_type = other DNA
cataatgccaggcgggccatttac

SEQ ID NO: 41 - DNA, 20 bp, synthetic construct
  misc_feature 1..20: note = Forward primer for qPCR of mouse GAPDH
  source 1..20: mol_type = other DNA
ccactcacggcaaattcaac

SEQ ID NO: 42 - DNA, 21 bp, synthetic construct
  misc_feature 1..21: note = Reverse primer for qPCR of mouse GAPDH
  source 1..21: mol_type = other DNA
accagtagactccacgacata

SEQ ID NO: 43 - DNA, 24 bp, synthetic construct
  misc_feature 1..24: note = Probe for qPCR of mouse GAPDH
  source 1..24: mol_type = other DNA
ttgtcatcaacgggaagcccatca

SEQ ID NO: 44 - DNA, 21 bp, synthetic construct
  misc_feature 1..21: note = Forward primer for qPCR of CoSMN11
  source 1..21: mol_type = other DNA
ctatcatccctcctcctcctc

SEQ ID NO: 45 - DNA, 20 bp, synthetic construct
  misc_feature 1..20: note = Reverse primer for qPCR of CoSMN11
  source 1..20: mol_type = other DNA
gcctgaagcccatgtagtag

SEQ ID NO: 46 - DNA, 24 bp, synthetic construct
  misc_feature 1..24: note = Probe for qPCR of CoSMN11
  source 1..24: mol_type = other DNA
ctggtacatgagcggctaccacac

(19) United States
(12) Patent Application Publication
(10) Pub. No.: US 2025/0369012 A1
(43) Pub. Date: Dec. 4, 2025

(54) RECOMBINANT AAV FOR THE GENE THERAPY OF SMA DISEASE

(71) Applicant: Skyline Therapeutics Limited, Cayman Islands (GB)

(72) Inventors: Yanqun SHU, Shanghai (CN); Carol HUANG, Shanghai (CN); Jinzhao HOU, Shanghai (CN); Wenting ZHU, Shanghai (CN); Ruojie WANG, Shanghai (CN); Yu WU, Shanghai (CN); Lingling ZHAO, Shanghai (CN)

(73) Assignee: Skyline Therapeutics Limited, Cayman Islands (GB)

(21) Appl. No.: 18/877,654

(22) PCT Filed: Jun. 20, 2023
(86) PCT No.: PCT/CN2023/101244

§ 371 Date: Dec. 20, 2024

(30) Foreign Application Priority Data
Jun. 21, 2022 (WO) PCT/CN2022/100050

(51) Int. Cl. C12N 15/86; A61K 48/00; A61P 21/00

(52) U.S. Cl. CPC C12N 15/86; A61P 21/00; A61K 48/00; C12N 2750/14143

(57) ABSTRACT

The present invention provides a polynucleotide and recombinant AAV encoding hSMN1. The present invention also provides a method of treating SMA disease comprising administering the recombinant AAV to a subject in need thereof.

### TECHNICAL FIELD

The present invention is related to gene therapy. In particular, the present invention involves the recombinant adeno-associated virus (AAV) for the gene therapy of SMA disease.

### BACKGROUND

Spinal Muscular Atrophy (SMA) is an autosome recessive disorder resulted from mutations in the SMN1 gene on chromosome 5, which results in death of neurons in anterior horn of spinal cord, and subsequent atrophy. A prevalence of approximately 1-2 per 100,000 persons has been reported with type I SMA. The prevalence in China is estimated to 1.2k-1.5k type I cases and 20k cases for other subtypes.

Type I SMA is a devastating disease. Patients' age of onset is 0-6 months, and they can never sit. The life-span is normally less than 2 years.

Several therapies for SMA have been approved, including i) a gene therapy (Zolgensma), which was approved by the U.S. FDA on May 24, 2019 for the treatment of pediatric patients less than 2 years old, and was also approved by the EMA in May 2020; ii) Nusinersen (antisense oligonucleotide, ASO), which was approved by FDA in December 2016 and by EMA in May 2017; and iii) Evrysdi, which was approved by FDA in August 2020. However, the approved therapies have some drawbacks limiting the wide use of them.

Zolgensman is very expensive for sure (about 2 million USD/IV injection). High dose injection may cause high levels of liver enzymes, which can lead to serious liver damage (https://www.medicalnewstoday.com/articles/drugs-zolgensma).

Nusinersen is directly delivered to the central nervous system (CNS) intrathecally (that is, through a lumbar puncture for delivery into the cerebrospinal fluid, to reach targets in the central nervous system where motor neuron loss begins). After 4 initial loading doses, Nusinersen is given 3 times a year (https://www.spinraza.com and https://medlineplus.gov/druginfo/meds/a617010.html). Nusinersen requires continuous administration in a traumatic and complicated manner with a higher risky, and needs well trained doctor/nurse to perform the operation.

Evrysdi is supplied as an oral solution, but needs to be administered once daily after a meal, and must be constituted by a pharmacist prior to dispensing. Evrysdi can be administered orally using the provided oral syringe. If the patient is unable to swallow and has a nasogastric or gastrostomy tube, Evrysdi can be administered via the tube. The tube should be flushed with water after delivering Evrysdi (https://www.centerwatch.com/directories/1067-fda-approved-drugs/listing/4644-evrysdi-risdiplam). The administration of Evrysdi is inconvenient. The young patients (2 years old or less) or disabled patients need help from parents/nurses because they apparently cannot orally taken by themselves.

Therefore, there is a need of a new therapy for SMA especially Type I SMA, such as a gene therapy that can provided persistent expression of functional SMN1 at a desired level.

### SUMMARY OF INVENTION

In order to meet the need, the inventors have developed a gene therapy for SMA disease which offers a persistent and endogenous production of SMN1 following the transfer of a functional copy of the SMN1 gene such as a SMN1 gene encoding a wild-type SMN1 polypeptide to a patient.

In a first aspect, the present invention provides a polynucleotide of interest comprising a nucleotide sequence selected from the group consisting of SEQ ID NOs: 2, 3, 4, 5, 6, 7, 8, 9, 10, 11, 12, 13, 14, 15, 16, 17, 18, 19 and 20.

In a second aspect, the present invention provides an expression construct comprising a polynucleotide of interest that comprises a nucleotide sequence selected from the group consisting of SEQ ID NOs: 2, 3, 4, 5, 6, 7, 8, 9, 10, 11, 12, 13, 14, 15, 16, 17, 18, 19 and 20, and is operably linked to a promoter. In some embodiments, the nucleotide sequence is SEQ ID NO: 11.

In some embodiments, the construct further comprises an intron. In some embodiments, the intron is between the promoter and the polynucleotide of interest. In some embodiments, the intron comprises a nucleotide sequence selected from the group consisting of SEQ ID NOs: 24 and 29. In some embodiments, the construct further comprises an enhancer. In some embodiments, the enhancer is upstream of the promoter. In some embodiments, the enhancer comprises a nucleotide sequence set forth in SEQ ID NO: 22. In some embodiments, the promoter comprises a nucleotide sequence selected from a group consisting of SEQ ID NOs: 23, 28 and 31.

In a third aspect, the present invention provides a recombinant adeno-associated virus (rAAV) comprising a genome comprising the expression construct of the present invention. In some embodiments, the rAAV is prepared by a system containing a transgene plasmid comprising the genome of the rAAV, a packaging plasmid encoding the REP and/or CAP proteins, and a helper plasmid. Therefore, the present invention further provides a vector comprising the expression construct of the invention.

In a fourth aspect, the present invention provides a pharmaceutical composition comprising the rAAV of the present invention.

In a fifth aspect, the present invention provides a host cell comprising the polynucleotide, the expression construct or the rAAV of the present invention.

In a sixth aspect, the present invention provides a method of treating a disease associated with the deficiency of SMN1, comprising administering the rAAV or the pharmaceutical composition of the present invention to a subject in need thereof.

The present invention also provides use of the polynucleotide, the expression construct, the rAAV, the pharmaceutical composition and/or the host cell of the present invention in the preparation of a medicament for treating a disease associated with the deficiency of SMN1 in a subject in need thereof.

In some embodiments, the disease is SMA, such as Type I SMA.

### BRIEF DESCRIPTION OF THE DRAWINGS

FIG. 1 shows the structures of the constructs of the invention (A), the GFP control construct (B) and the constructs of Zolgensma (C) and Zolgensma GFP (D).

FIG. 2 shows the maps of the transgene plasmid (A), helper plasmid (B), packaging plasmid (C) and fLuc2 plasmid (D).

FIG. 3 shows the Western Blot for detecting the expression of hSMN1 by the transgene plasmids in 293T cell, alpha tubulin was used as reference. Lanes 2-20 represent samples from cells transfected with the transgene plasmids comprising CoSMN2-20, respectively; Lanes C represent samples from non-transfected cells; Lanes Zol represent samples from cells transfected with the transgene plasmids comprising the Zolgensma construct; and Lanes GFP represent samples from cells transfected with the transgene plasmids encoding GFP.

FIG. 4 shows the luciferase activity in the transfected cells, columns 2-20 represent samples from cells co-transfected with the transgene plasmids comprising CoSMN2-20, respectively; columns “cells” represent samples from non-transfected cells; columns Zol represent samples from cells co-transfected with the transgene plasmids comprising the Zolgensma construct; and columns GFP represent samples from cells co-transfected with the transgene plasmids encoding GFP.

FIG. 5 shows the relative hSMN1 expression in 293T cells, the results of the Western Blot was corrected by luciferase activity, and normalized to the Zol samples.

FIG. 6 shows the relative hSMN1 expression in 293T cells by the rAAVs of the invention.

FIG. 7 shows the relative hSMN1 expression in wildtype mice by the rAAVs of the invention.

FIG. 8 shows the relative hSMN1 expression in SMA models by the rAAVs of the invention.

FIG. 9 shows the relative hSMN1 expression in wildtype mice by the rAAVs of the invention comprising various combinations of promoter and intron.

### DETAILED DESCRIPTION

### 1. Definitions

Unless otherwise indicated, all terms used herein have the same meaning as they would to one skilled in the art, and the practice of the present invention will employ conventional techniques of microbiology and recombinant DNA technology, which are within the knowledge of those of skill in the art.

As used herein, “SMN1”, when referring to a gene, means survival motor neuron gene 1, and means the protein encoded by the SMN1 gene when referring to a protein. “hSMN1” means human “SMN1” gene or protein.

Adeno-associated virus (AAV) is a member of Parvoviridac family. It is a simple single-stranded DNA virus, and requires a helper virus (such as adenovirus) for replication. The genome of a wildtype AAV contains approximately 4.7 kilobases (kb), comprising the cap and rep genes between two inverted terminal repeat (ITR) sequences, approximately 145 nucleotides in length, with interrupted palindromic sequences that can fold into hairpin structures that function as primers during initiation of DNA replication. The cap gene encodes the viral capsid protein, and the rep gene is involved in the replication and integration of AAV. AAV can infect a variety of cells, and the viral DNA can be integrated into human chromosome 19 in the presence of the rep product.

As used herein, the term “inverted terminal repeats” or “ITRs” as used herein refers to AAV viral cis-elements named due to their symmetry. These elements are essential for efficient multiplication of an AAV genome. In the present invention, the term “ITR” refers to ITRs of known natural AAV serotypes, to chimeric ITRs formed by the fusion of ITR elements derived from different serotypes, and to functional variant thereof.

The production of a recombinant AAV particle may involve three plasmids, a transgene plasmid comprising an expression construct for expressing an exogenous polynucleotide, a packaging plasmid encoding the REP and/or CAP proteins, and a helper plasmid.

As used herein, the term “expression construct” refers to a single-stranded or double-stranded polynucleotide, which is isolated from a naturally occurring gene or modified to contain a nucleic acid segment that does not naturally occur. The expression construct may contain the control sequences required to express the coding sequence of the present invention.

As used herein, the term “polynucleotide” usually refers to generally a nucleic acid molecule (e.g., 100 bases and up to 30 kilobases in length) and a sequence that is either complementary (antisense) or identical (sense) to the sequence of a messenger RNA (mRNA) or miRNA fragment or molecule. The term can also refer to DNA or RNA molecules that are either transcribed or non-transcribed.

The term “exogenous polynucleotide” as used herein refers to a nucleotide sequence that does not originate from the host in which it is placed. It may be identical to the host's DNA or heterologous. An example is a sequence of interest inserted into a vector. Such exogenous DNA sequences may be derived from a variety of sources including DNA, cDNA, synthetic DNA, and RNA. Exogenous polynucleotides also encompass DNA sequences that encode antisense oligonucleotides.

As used herein, the term “expression” includes any step involved in the production of a polypeptide, including but not limited to transcription, post-transcriptional modification, translation, post-translational modification, and secretion.

A “control sequence” includes all elements necessary or beneficial for the expression of the polynucleotide encoding the polypeptide of the present invention. Each control sequence may be natural or foreign to the nucleotide sequence encoding the polypeptide, or natural or foreign to each other. Such control sequences include, but are not limited to, leader sequence, polyadenylation sequence, propeptide sequence, promoter, enhancer, signal peptide sequence, and transcription terminator. At a minimum, control sequences include a promoter and signals for the termination of transcription and translation.

For example, the control sequence may be a suitable promoter sequence, a nucleotide sequence recognized by the host cell to express the polynucleotide encoding the polypeptide of the present invention. The promoter sequence contains a transcription control sequence that mediates the expression of the polypeptide. The promoter may be any nucleotide sequence that exhibits transcriptional activity in the selected host cell, for example, lac operon of E. coli. The promoters also include mutant, modified and hybrid promoters, and can be obtained from genes encoding extracellular or intracellular polypeptides, which are homologous or heterologous to the host cell.

In some cases, an intron can be included in the construct to improve the expression of the coding sequence. A “modified” intron comprises a modification such as substitution, insertion or deletion of one or more nucleotides in an internal region of an initial intron.

As used herein, the term “operably linked” herein refers to a configuration in which a control sequence is placed at an appropriate position relative to the coding sequence of the polynucleotide sequence, whereby the control sequence directs the expression of the polypeptide coding sequence.

The polynucleotide encoding the SMN1 protein can be subjected to various manipulations to improve the expression of the polypeptide. Before the insertion thereof into a vector, manipulation of the polynucleotide according to the expression vector or the host, such as codon optimization, is desirable or necessary.

The term “recombinant” as used herein refers to nucleic acids, vectors, polypeptides, or proteins that have been generated using DNA recombination (cloning) methods and are distinguishable from native or wild-type nucleic acids, vectors, polypeptides, or proteins.

The terms “polypeptide” and “protein” are used interchangeably herein and refer to a polymer of amino acids and includes full-length proteins and fragments thereof.

As used herein, the term “host cell” refers to, for example microorganisms, yeast cells, insect cells, and mammalian cells, that can be, or have been, used as recipients of rAAV vectors. The term includes the progeny of the original cell which has been transduced. Thus, a “host cell” as used herein generally refers to a cell which has been transduced with an exogenous DNA sequence. It is understood that the progeny of a single parental cell may not necessarily be completely identical in morphology or in genomic or total DNA complement to the original parent, due to natural, accidental, or deliberate mutation.

The term “pharmaceutically acceptable” as used herein refers to molecular entities and compositions that are physiologically tolerable and do not typically produce toxicity or an allergic or similar untoward reaction, such as gastric upset, dizziness and the like, when administered to a human.

The term “subject” as used herein includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

### 2. The Polynucleotide or Construct for Expressing hSMN1

Gene therapy aims to correct defective genes that underlie the development of diseases, and to introduce exogenous gene into the cell of interest in a subject to express the product of the exogenous gene that is useful for treating a certain disease, such as SMA disease. A common approach for this purpose involves the delivery of a functional gene such as SMN1 to the nucleus. This gene may then be inserted into the genome of the cell of interest or may remain episomal. Delivery of a functional gene to a subject's target cells can be carried out via numerous methods, including the use of viral vectors. Among the many viral vectors available (e.g, retrovirus, lentivirus, adenovirus, and the like), AAV is gaining popularity as a versatile vector in gene therapy.

Vectors derived from AAV are particularly attractive for delivering genetic material because (i) they are able to infect (transduce) a wide variety of non-dividing and dividing cell types including muscle fibers and neurons; (ii) they are devoid of the virus structural genes, thereby eliminating the natural host cell responses to virus infection, e.g., interferon-mediated responses; (iii) wild-type viruses have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected). AAV vectors can also be produced at high titer and it has been reported that intra-arterial, intra-venous, or intra-peritoneal injections allow gene transfer to significant muscle regions in rodents through a single injection.

The present invention thus intends to provide a polynucleotide of interest, an expression construct, or a vector comprising the polynucleotide of interest, for expressing SMN1 in a subject. In some embodiments, the SMN1 can be from any animal species, including but not limited to, human, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like.

In some embodiments, the SMN1 is human SMN1. In some embodiments, the hSMN1 comprise the amino acid sequence of SEQ ID NO: 21 or an allelic variant thereof. In some embodiments, the SMN1 is a functional SMN1 polypeptide, such as a SMN1 polypeptide having at least 10%, 20%, 30%, 40%, 50%, 60% 70%, 80%, 90%, 100% or more activity of the polypeptide of SEQ ID NO: 21.

In some embodiments, the polynucleotide encoding SMN1 is codon-optimized to improve the expression.

In some embodiments, the polynucleotide comprises a nucleotide sequence selected from SEQ ID NOs: 2, 3, 4, 5, 6, 7, 8, 9, 10, 11, 12, 13, 14, 15, 16, 17, 18, 19 and 20. Surprisingly, the inventors found that the polynucleotide with a nucleotide sequence of SEQ ID NOs: 4, 5, 6, 7, 8, 11, 16 and 19 achieves a higher expression of hSMN1 (1.5-4 folds) than SEQ ID NO: 1 in cells; while in the in vivo tests, SEQ ID NOs: 3, 6, 8 and 11 achieves higher expression in spinal cord, but lower expression in heart and liver, as compared to SEQ ID NO: 1.

In some embodiments, the polynucleotide comprises SEQ ID NO: 2 or a nucleotide sequence at least 80%, 85%, 90%, 95%, 96%, 97%, 98%, or 99% identical to SEQ ID NO: 2. In some embodiments, the polynucleotide comprises a nucleotide sequence at least 80%, 85%, 90%, 95%, 96%, 97%, 98%, or 99% identical to SEQ ID NO: 2, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 2, and encodes the polypeptide of SEQ ID NO: 21 or an allelic variant thereof.

In some embodiments, the polynucleotide comprises SEQ ID NO: 3 or a nucleotide sequence at least 80%, 85%, 90%, 95%, 96%, 97%, 98%, or 99% identical to SEQ ID NO: 3. In some embodiments, the polynucleotide comprises a nucleotide sequence at least 80%, 85%, 90%, 95%, 96%, 97%, 98%, or 99% identical to SEQ ID NO: 3, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 3, and encodes the polypeptide of SEQ ID NO: 21 or an allelic variant thereof.

In some embodiments, the polynucleotide comprises SEQ ID NO: 4 or a nucleotide sequence at least 80%, 85%, 90%, 95%, 96%, 97%, 98%, or 99% identical to SEQ ID NO: 4. In some embodiments, the polynucleotide comprises a nucleotide sequence at least 80%, 85%, 90%, 95%, 96%, 97%, 98%, or 99% identical to SEQ ID NO: 4, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 4 and encodes the polypeptide of SEQ ID NO: 21 or an allelic variant thereof.

In some embodiments, the polynucleotide comprises SEQ ID NO: 5 or a nucleotide sequence at least 80%, 85%, 90%, 95%, 96%, 97%, 98%, or 99% identical to SEQ ID NO: 5. In some embodiments, the polynucleotide comprises a nucleotide sequence at least 80%, 85%, 90%, 95%, 96%, 97%, 98%, or 99% identical to SEQ ID NO: 5, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 5, and encodes the polypeptide of SEQ ID NO: 21 or an allelic variant thereof.

In some embodiments, the polynucleotide comprises SEQ ID NO: 6 or a nucleotide sequence at least 80%, 85%, 90%, 95%, 96%, 97%, 98%, or 99% identical to SEQ ID NO: 6. In some embodiments, the polynucleotide comprises a nucleotide sequence at least 80%, 85%, 90%, 95%, 96%, 97%, 98%, or 99% identical to SEQ ID NO: 6, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 6, and encodes the polypeptide of SEQ ID NO: 21 or an allelic variant thereof.

In some embodiments, the polynucleotide comprises SEQ ID NO: 7 or a nucleotide sequence at least 80%, 85%, 90%, 95%, 96%, 97%, 98%, or 99% identical to SEQ ID NO: 7. In some embodiments, the polynucleotide comprises a nucleotide sequence at least 80%, 85%, 90%, 95%, 96%, 97%, 98%, or 99% identical to SEQ ID NO: 7, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 7, and encodes the polypeptide of SEQ ID NO: 21 or an allelic variant thereof.

In some embodiments, the polynucleotide comprises SEQ ID NO: 8 or a nucleotide sequence at least 80%, 85%, 90%, 95%, 96%, 97%, 98%, or 99% identical to SEQ ID NO: 8. In some embodiments, the polynucleotide comprises a nucleotide sequence at least 80%, 85%, 90%, 95%, 96%, 97%, 98%, or 99% identical to SEQ ID NO: 8, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 8, and encodes the polypeptide of SEQ ID NO: 21 or an allelic variant thereof.

In some embodiments, the polynucleotide comprises SEQ ID NO: 9 or a nucleotide sequence at least 80%, 85%, 90%, 95%, 96%, 97%, 98%, or 99% identical to SEQ ID NO: 9. In some embodiments, the polynucleotide comprises a nucleotide sequence at least 80%, 85%, 90%, 95%, 96%, 97%, 98%, or 99% identical to SEQ ID NO: 9, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 9, and encodes the polypeptide of SEQ ID NO: 21 or an allelic variant thereof.

In some embodiments, the polynucleotide comprises SEQ ID NO: 10 or a nucleotide sequence at least 80%, 85%, 90%, 95%, 96%, 97%, 98%, or 99% identical to SEQ ID NO: 10. In some embodiments, the polynucleotide comprises a nucleotide sequence at least 80%, 85%, 90%, 95%, 96%, 97%, 98%, or 99% identical to SEQ ID NO: 10, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 10, and encodes the polypeptide of SEQ ID NO: 21 or an allelic variant thereof.

In some embodiments, the polynucleotide comprises SEQ ID NO: 11 or a nucleotide sequence at least 80%, 85%, 90%, 95%, 96%, 97%, 98%, or 99% identical to SEQ ID NO: 11. In some embodiments, the polynucleotide comprises a nucleotide sequence at least 80%, 85%, 90%, 95%, 96%, 97%, 98%, or 99% identical to SEQ ID NO: 11, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 11, and encodes the polypeptide of SEQ ID NO: 21 or an allelic variant thereof.

In some embodiments, the polynucleotide comprises SEQ ID NO: 12 or a nucleotide sequence at least 80%, 85%, 90%, 95%, 96%, 97%, 98%, or 99% identical to SEQ ID NO: 12. In some embodiments, the polynucleotide comprises a nucleotide sequence at least 80%, 85%, 90%, 95%, 96%, 97%, 98%, or 99% identical to SEQ ID NO: 12, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 12, and encodes the polypeptide of SEQ ID NO: 21 or an allelic variant thereof.

In some embodiments, the polynucleotide comprises SEQ ID NO: 13 or a nucleotide sequence at least 80%, 85%, 90%, 95%, 96%, 97%, 98%, or 99% identical to SEQ ID NO: 13. In some embodiments, the polynucleotide comprises a nucleotide sequence at least 80%, 85%, 90%, 95%, 96%, 97%, 98%, or 99% identical to SEQ ID NO: 13, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 13, and encodes the polypeptide of SEQ ID NO: 21 or an allelic variant thereof

In some embodiments, the polynucleotide comprises SEQ ID NO: 14 or a nucleotide sequence at least 80%, 85%, 90%, 95%, 96%, 97%, 98%, or 99% identical to SEQ ID NO: 14. In some embodiments, the polynucleotide comprises a nucleotide sequence at least 80%, 85%, 90%, 95%, 96%, 97%, 98%, or 99% identical to SEQ ID NO: 14, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 14, and encodes the polypeptide of SEQ ID NO: 21 or an allelic variant thereof.

In some embodiments, the polynucleotide comprises SEQ ID NO: 15 or a nucleotide sequence at least 80%, 85%, 90%, 95%, 96%, 97%, 98%, or 99% identical to SEQ ID NO: 15. In some embodiments, the polynucleotide comprises a nucleotide sequence at least 80%, 85%, 90%, 95%, 96%, 97%, 98%, or 99% identical to SEQ ID NO: 15, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 15, and encodes the polypeptide of SEQ ID NO: 21 or an allelic variant thereof.

In some embodiments, the polynucleotide comprises SEQ ID NO: 16 or a nucleotide sequence at least 80%, 85%, 90%, 95%, 96%, 97%, 98%, or 99% identical to SEQ ID NO: 16. In some embodiments, the polynucleotide comprises a nucleotide sequence at least 80%, 85%, 90%, 95%, 96%, 97%, 98%, or 99% identical to SEQ ID NO: 16, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 16, and encodes the polypeptide of SEQ ID NO: 21 or an allelic variant thereof.

In some embodiments, the polynucleotide comprises SEQ ID NO: 17 or a nucleotide sequence at least 80%, 85%, 90%, 95%, 96%, 97%, 98%, or 99% identical to SEQ ID NO: 17. In some embodiments, the polynucleotide comprises a nucleotide sequence at least 80%, 85%, 90%, 95%, 96%, 97%, 98%, or 99% identical to SEQ ID NO: 17, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 17, and encodes the polypeptide of SEQ ID NO: 21 or an allelic variant thereof.

In some embodiments, the polynucleotide comprises SEQ ID NO: 18 or a nucleotide sequence at least 80%, 85%, 90%, 95%, 96%, 97%, 98%, or 99% identical to SEQ ID NO: 18. In some embodiments, the polynucleotide comprises a nucleotide sequence at least 80%, 85%, 90%, 95%, 96%, 97%, 98%, or 99% identical to SEQ ID NO: 18, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 18, and encodes the polypeptide of SEQ ID NO: 21 or an allelic variant thereof.

In some embodiments, the polynucleotide comprises SEQ ID NO: 19 or a nucleotide sequence at least 80%, 85%, 90%, 95%, 96%, 97%, 98%, or 99% identical to SEQ ID NO: 19. In some embodiments, the polynucleotide comprises a nucleotide sequence at least 80%, 85%, 90%, 95%, 96%, 97%, 98%, or 99% identical to SEQ ID NO: 19, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 19, and encodes the polypeptide of SEQ ID NO: 21 or an allelic variant thereof.

In some embodiments, the polynucleotide comprises SEQ ID NO: 20 or a nucleotide sequence at least 80%, 85%, 90%, 95%, 96%, 97%, 98%, or 99% identical to SEQ ID NO: 20. In some embodiments, the polynucleotide comprises a nucleotide sequence at least 80%, 85%, 90%, 95%, 96%, 97%, 98%, or 99% identical to SEQ ID NO: 20, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 20, and encodes the polypeptide of SEQ ID NO: 21 or an allelic variant thereof.

The present invention also provides an expression construct for expressing SMN1, which comprises a polynucleotide of interest encoding SMN1 operably linked to a promoter. In some embodiments, the SMN1 is human SMN1. In some embodiments, the hSMN1 comprises the amino acid sequence of SEQ ID NO: 21 or an allelic variant thereof.

In some embodiment, the expression construct achieves a higher expression of SMN1 polypeptide and/or mRNA in spinal cord, and/or a lower expression of SMN1 polypeptide and/or mRNA in non-spinal tissues, such as heart and/or liver, as compared to a control expression construct, e.g., that comprises a nucleotide sequence of SEQ ID NO: 1.

In some embodiments, the polynucleotide comprises a nucleotide sequence selected from SEQ ID NOs: 2, 3, 4, 5, 6, 7, 8, 9, 10, 11, 12, 13, 14, 15, 16, 17, 18, 19 and 20. Surprisingly, the inventors found that the polynucleotide with a nucleotide sequence of SEQ ID NOs: 4, 5, 6, 7, 8, 11, 16 and 19 achieves a higher expression of hSMN1 (1.5-4 folds) than SEQ ID NO: 1 in cells; while in the in vivo tests, SEQ ID NOs: 3, 6, 8 and 11 achieves higher expression in spinal cord, but lower expression in heart and liver, as compared to SEQ ID NO: 1.

In some embodiments, the polynucleotide comprises SEQ ID NO: 2 or a nucleotide sequence at least 80%, 85%, 90%, 95%, 96%, 97%, 98%, or 99% identical to SEQ ID NO: 2. In some embodiments, the polynucleotide comprises a nucleotide sequence at least 80%, 85%, 90%, 95%, 96%, 97%, 98%, or 99% identical to SEQ ID NO: 2, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 2, and encodes the polypeptide of SEQ ID NO: 21 or an allelic variant thereof.

In some embodiments, the polynucleotide comprises SEQ ID NO: 3 or a nucleotide sequence at least 80%, 85%, 90%, 95%, 96%, 97%, 98%, or 99% identical to SEQ ID NO: 3. In some embodiments, the polynucleotide comprises a nucleotide sequence at least 80%, 85%, 90%, 95%, 96%, 97%, 98%, or 99% identical to SEQ ID NO: 3, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 3, and encodes the polypeptide of SEQ ID NO: 21 or an allelic variant thereof.

In some embodiments, the polynucleotide comprises SEQ ID NO: 4 or a nucleotide sequence at least 80%, 85%, 90%, 95%, 96%, 97%, 98%, or 99% identical to SEQ ID NO: 4. In some embodiments, the polynucleotide comprises a nucleotide sequence at least 80%, 85%, 90%, 95%, 96%, 97%, 98%, or 99% identical to SEQ ID NO: 4, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 4 and encodes the polypeptide of SEQ ID NO: 21 or an allelic variant thereof.

In some embodiments, the polynucleotide comprises SEQ ID NO: 5 or a nucleotide sequence at least 80%, 85%, 90%, 95%, 96%, 97%, 98%, or 99% identical to SEQ ID NO: 5. In some embodiments, the polynucleotide comprises a nucleotide sequence at least 80%, 85%, 90%, 95%, 96%, 97%, 98%, or 99% identical to SEQ ID NO: 5, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 5, and encodes the polypeptide of SEQ ID NO: 21 or an allelic variant thereof.

In some embodiments, the polynucleotide comprises SEQ ID NO: 6 or a nucleotide sequence at least 80%, 85%, 90%, 95%, 96%, 97%, 98%, or 99% identical to SEQ ID NO: 6. In some embodiments, the polynucleotide comprises a nucleotide sequence at least 80%, 85%, 90%, 95%, 96%, 97%, 98%, or 99% identical to SEQ ID NO: 6, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 6, and encodes the polypeptide of SEQ ID NO: 21 or an allelic variant thereof.

In some embodiments, the polynucleotide comprises SEQ ID NO: 7 or a nucleotide sequence at least 80%, 85%, 90%, 95%, 96%, 97%, 98%, or 99% identical to SEQ ID NO: 7. In some embodiments, the polynucleotide comprises a nucleotide sequence at least 80%, 85%, 90%, 95%, 96%, 97%, 98%, or 99% identical to SEQ ID NO: 7, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 7, and encodes the polypeptide of SEQ ID NO: 21 or an allelic variant thereof.

In some embodiments, the polynucleotide comprises SEQ ID NO: 8 or a nucleotide sequence at least 80%, 85%, 90%, 95%, 96%, 97%, 98%, or 99% identical to SEQ ID NO: 8. In some embodiments, the polynucleotide comprises a nucleotide sequence at least 80%, 85%, 90%, 95%, 96%, 97%, 98%, or 99% identical to SEQ ID NO: 8, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 8, and encodes the polypeptide of SEQ ID NO: 21 or an allelic variant thereof.

In some embodiments, the polynucleotide comprises SEQ ID NO: 9 or a nucleotide sequence at least 80%, 85%, 90%, 95%, 96%, 97%, 98%, or 99% identical to SEQ ID NO: 9. In some embodiments, the polynucleotide comprises a nucleotide sequence at least 80%, 85%, 90%, 95%, 96%, 97%, 98%, or 99% identical to SEQ ID NO: 9, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 9, and encodes the polypeptide of SEQ ID NO: 21 or an allelic variant thereof.

In some embodiments, the polynucleotide comprises SEQ ID NO: 10 or a nucleotide sequence at least 80%, 85%, 90%, 95%, 96%, 97%, 98%, or 99% identical to SEQ ID NO: 10. In some embodiments, the polynucleotide comprises a nucleotide sequence at least 80%, 85%, 90%, 95%, 96%, 97%, 98%, or 99% identical to SEQ ID NO: 10, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 10, and encodes the polypeptide of SEQ ID NO: 21 or an allelic variant thereof.

In some embodiments, the polynucleotide comprises SEQ ID NO: 11 or a nucleotide sequence at least 80%, 85%, 90%, 95%, 96%, 97%, 98%, or 99% identical to SEQ ID NO: 11. In some embodiments, the polynucleotide comprises a nucleotide sequence at least 80%, 85%, 90%, 95%, 96%, 97%, 98%, or 99% identical to SEQ ID NO: 11, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 11, and encodes the polypeptide of SEQ ID NO: 21 or an allelic variant thereof.

In some embodiments, the polynucleotide comprises SEQ ID NO: 12 or a nucleotide sequence at least 80%, 85%, 90%, 95%, 96%, 97%, 98%, or 99% identical to SEQ ID NO: 12. In some embodiments, the polynucleotide comprises a nucleotide sequence at least 80%, 85%, 90%, 95%, 96%, 97%, 98%, or 99% identical to SEQ ID NO: 12, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 12, and encodes the polypeptide of SEQ ID NO: 21 or an allelic variant thereof.

In some embodiments, the polynucleotide comprises SEQ ID NO: 13 or a nucleotide sequence at least 80%, 85%, 90%, 95%, 96%, 97%, 98%, or 99% identical to SEQ ID NO: 13. In some embodiments, the polynucleotide comprises a nucleotide sequence at least 80%, 85%, 90%, 95%, 96%, 97%, 98%, or 99% identical to SEQ ID NO: 13, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 13, and encodes the polypeptide of SEQ ID NO: 21 or an allelic variant thereof.

In some embodiments, the polynucleotide comprises SEQ ID NO: 14 or a nucleotide sequence at least 80%, 85%, 90%, 95%, 96%, 97%, 98%, or 99% identical to SEQ ID NO: 14. In some embodiments, the polynucleotide comprises a nucleotide sequence at least 80%, 85%, 90%, 95%, 96%, 97%, 98%, or 99% identical to SEQ ID NO: 14, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 14, and encodes the polypeptide of SEQ ID NO: 21 or an allelic variant thereof.

In some embodiments, the polynucleotide comprises SEQ ID NO: 15 or a nucleotide sequence at least 80%, 85%, 90%, 95%, 96%, 97%, 98%, or 99% identical to SEQ ID NO: 15. In some embodiments, the polynucleotide comprises a nucleotide sequence at least 80%, 85%, 90%, 95%, 96%, 97%, 98%, or 99% identical to SEQ ID NO: 15, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 15, and encodes the polypeptide of SEQ ID NO: 21 or an allelic variant thereof.

In some embodiments, the polynucleotide comprises SEQ ID NO: 16 or a nucleotide sequence at least 80%, 85%, 90%, 95%, 96%, 97%, 98%, or 99% identical to SEQ ID NO: 16. In some embodiments, the polynucleotide comprises a nucleotide sequence at least 80%, 85%, 90%, 95%, 96%, 97%, 98%, or 99% identical to SEQ ID NO: 16, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 16, and encodes the polypeptide of SEQ ID NO: 21 or an allelic variant thereof.

In some embodiments, the polynucleotide comprises SEQ ID NO: 17 or a nucleotide sequence at least 80%, 85%, 90%, 95%, 96%, 97%, 98%, or 99% identical to SEQ ID NO: 17. In some embodiments, the polynucleotide comprises a nucleotide sequence at least 80%, 85%, 90%, 95%, 96%, 97%, 98%, or 99% identical to SEQ ID NO: 17, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 17, and encodes the polypeptide of SEQ ID NO: 21 or an allelic variant thereof.

In some embodiments, the polynucleotide comprises SEQ ID NO: 18 or a nucleotide sequence at least 80%, 85%, 90%, 95%, 96%, 97%, 98%, or 99% identical to SEQ ID NO: 18. In some embodiments, the polynucleotide comprises a nucleotide sequence at least 80%, 85%, 90%, 95%, 96%, 97%, 98%, or 99% identical to SEQ ID NO: 18, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 18, and encodes the polypeptide of SEQ ID NO: 21 or an allelic variant thereof.

In some embodiments, the polynucleotide comprises SEQ ID NO: 19 or a nucleotide sequence at least 80%, 85%, 90%, 95%, 96%, 97%, 98%, or 99% identical to SEQ ID NO: 19. In some embodiments, the polynucleotide comprises a nucleotide sequence at least 80%, 85%, 90%, 95%, 96%, 97%, 98%, or 99% identical to SEQ ID NO: 19, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 19, and encodes the polypeptide of SEQ ID NO: 21 or an allelic variant thereof.

In some embodiments, the polynucleotide comprises SEQ ID NO: 20 or a nucleotide sequence at least 80%, 85%, 90%, 95%, 96%, 97%, 98%, or 99% identical to SEQ ID NO: 20. In some embodiments, the polynucleotide comprises a nucleotide sequence at least 80%, 85%, 90%, 95%, 96%, 97%, 98%, or 99% identical to SEQ ID NO: 20, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 20, and encodes the polypeptide of SEQ ID NO: 21 or an allelic variant thereof.

The construct will generally be transferred to mammalian cells (such as human cells) for expression. Such constructs often include promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene.

In some embodiments, the promoter is a constitutive promoter. In some embodiments, the promoter comprises SEQ ID NO: 23 or a nucleotide sequence at least 70%, 75%, 80%, 85%, 90%, 95%, 96%, 97%, 98%, or 99% identical to SEQ ID NO: 23. In some embodiments, the promoter comprises SEQ ID NO: 28 or a nucleotide sequence at least 70%, 75%, 80%, 85%, 90%, 95%, 96%, 97%, 98%, or 99% identical to SEQ ID NO: 28. In some embodiments, the promoter comprises SEQ ID NO: 31 or a nucleotide sequence at least 70%, 75%, 80%, 85%, 90%, 95%, 96%, 97%, 98%, or 99% identical to SEQ ID NO: 31.

In some embodiments, the expression construct further comprises an enhancer. In some embodiments, the enhancer is upstream of the promoter. In some embodiments, the enhancer is a cytomegalovirus (CMV) early enhancer. In some embodiments, the enhancer comprises SEQ ID NO: 22, or a nucleotide sequence at least 70%, 75%, 80%, 85%, 90%, 95%, 96%, 97%, 98%, or 99% identical to SEQ ID NO: 22.

It is also desirable to include an intron (e.g., an intron derived from a eukaryotic organism or an artificial intron) in the construct to increase the expression in eukaryotic cells. In some embodiments, the expression construct further comprises an intron. In some embodiments, the intron is upstream of the nucleotide sequence encoding SMN1. In some embodiments, the intron is downstream of the promoter.

In some embodiments, the intron is a PCI intron set forth in SEQ ID NO: 24. In some embodiments, the intron is chimeric intron (chicken beta-actin (CBA intron and exon)+rabbit globin intron) set forth in SEQ ID NO: 29.

In some embodiments, the intron comprises SEQ ID NO: 24 or 29, or a nucleotide sequence at least 70%, 75%, 80%, 85%, 90%, 95%, 96%, 97%, 98%, or 99% identical to SEQ ID NO: 24 or 29.

The expression construct further comprises a polyadenylation signal sequence for the processing of the transcript. In some embodiments, the construct comprises a polyadenylation signal sequence downstream of the nucleotide sequence encoding hSMN1. In some embodiments, the polyadenylation signal sequence comprises SEQ ID NO: 25 or 30, or a nucleotide sequence at least 70%, 75%, 80%, 85%, 90%, 95%, 96%, 97%, 98%, or 99% identical to SEQ ID NO: 25 or 30.

In some embodiments, the expression construct comprises, in the order of 5′ to 3′, an enhancer, a promoter, an intron, a polynucleotide of interest encoding hSMN1, and a polyadenylation signal sequence.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 2 or a nucleotide sequence at least 80%, 85%, 90%, 95%, 96%, 97%, 98%, or 99% identical to SEQ ID NO: 2, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 3 or a nucleotide sequence at least 80%, 85%, 90%, 95%, 96%, 97%, 98%, or 99% identical to SEQ ID NO: 3, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 4 or a nucleotide sequence at least 80%, 85%, 90%, 95%, 96%, 97%, 98%, or 99% identical to SEQ ID NO: 4, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 5 or a nucleotide sequence at least 80%, 85%, 90%, 95%, 96%, 97%, 98%, or 99% identical to SEQ ID NO: 5, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 6 or a nucleotide sequence at least 80%, 85%, 90%, 95%, 96%, 97%, 98%, or 99% identical to SEQ ID NO: 6, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 7 or a nucleotide sequence at least 80%, 85%, 90%, 95%, 96%, 97%, 98%, or 99% identical to SEQ ID NO: 7, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 8 or a nucleotide sequence at least 80%, 85%, 90%, 95%, 96%, 97%, 98%, or 99% identical to SEQ ID NO: 8, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 9 or a nucleotide sequence at least 80%, 85%, 90%, 95%, 96%, 97%, 98%, or 99% identical to SEQ ID NO: 9, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 10 or a nucleotide sequence at least 80%, 85%, 90%, 95%, 96%, 97%, 98%, or 99% identical to SEQ ID NO: 10, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 12 or a nucleotide sequence at least 80%, 85%, 90%, 95%, 96%, 97%, 98%, or 99% identical to SEQ ID NO: 12, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 13 or a nucleotide sequence at least 80%, 85%, 90%, 95%, 96%, 97%, 98%, or 99% identical to SEQ ID NO: 13, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 14 or a nucleotide sequence at least 80%, 85%, 90%, 95%, 96%, 97%, 98%, or 99% identical to SEQ ID NO: 14, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 15 or a nucleotide sequence at least 80%, 85%, 90%, 95%, 96%, 97%, 98%, or 99% identical to SEQ ID NO: 15, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 16 or a nucleotide sequence at least 80%, 85%, 90%, 95%, 96%, 97%, 98%, or 99% identical to SEQ ID NO: 16, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 17 or a nucleotide sequence at least 80%, 85%, 90%, 95%, 96%, 97%, 98%, or 99% identical to SEQ ID NO: 17, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 18 or a nucleotide sequence at least 80%, 85%, 90%, 95%, 96%, 97%, 98%, or 99% identical to SEQ ID NO: 18, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 19 or a nucleotide sequence at least 80%, 85%, 90%, 95%, 96%, 97%, 98%, or 99% identical to SEQ ID NO: 19, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 20 or a nucleotide sequence at least 80%, 85%, 90%, 95%, 96%, 97%, 98%, or 99% identical to SEQ ID NO: 20, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 2 or a nucleotide sequence at least 80%, 85%, 90%, 95%, 96%, 97%, 98%, or 99% identical to SEQ ID NO: 2, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 3 or a nucleotide sequence at least 80%, 85%, 90%, 95%, 96%, 97%, 98%, or 99% identical to SEQ ID NO: 3, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 4 or a nucleotide sequence at least 80%, 85%, 90%, 95%, 96%, 97%, 98%, or 99% identical to SEQ ID NO: 4, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 5 or a nucleotide sequence at least 80%, 85%, 90%, 95%, 96%, 97%, 98%, or 99% identical to SEQ ID NO: 5, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 6 or a nucleotide sequence at least 80%, 85%, 90%, 95%, 96%, 97%, 98%, or 99% identical to SEQ ID NO: 6, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 7 or a nucleotide sequence at least 80%, 85%, 90%, 95%, 96%, 97%, 98%, or 99% identical to SEQ ID NO: 7, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 8 or a nucleotide sequence at least 80%, 85%, 90%, 95%, 96%, 97%, 98%, or 99% identical to SEQ ID NO: 8, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 9 or a nucleotide sequence at least 80%, 85%, 90%, 95%, 96%, 97%, 98%, or 99% identical to SEQ ID NO: 9, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 10 or a nucleotide sequence at least 80%, 85%, 90%, 95%, 96%, 97%, 98%, or 99% identical to SEQ ID NO: 10, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 12 or a nucleotide sequence at least 80%, 85%, 90%, 95%, 96%, 97%, 98%, or 99% identical to SEQ ID NO: 12, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 13 or a nucleotide sequence at least 80%, 85%, 90%, 95%, 96%, 97%, 98%, or 99% identical to SEQ ID NO: 13, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 14 or a nucleotide sequence at least 80%, 85%, 90%, 95%, 96%, 97%, 98%, or 99% identical to SEQ ID NO: 14, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 15 or a nucleotide sequence at least 80%, 85%, 90%, 95%, 96%, 97%, 98%, or 99% identical to SEQ ID NO: 15, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 16 or a nucleotide sequence at least 80%, 85%, 90%, 95%, 96%, 97%, 98%, or 99% identical to SEQ ID NO: 16, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 17 or a nucleotide sequence at least 80%, 85%, 90%, 95%, 96%, 97%, 98%, or 99% identical to SEQ ID NO: 17, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 18 or a nucleotide sequence at least 80%, 85%, 90%, 95%, 96%, 97%, 98%, or 99% identical to SEQ ID NO: 18, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 19 or a nucleotide sequence at least 80%, 85%, 90%, 95%, 96%, 97%, 98%, or 99% identical to SEQ ID NO: 19, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 20 or a nucleotide sequence at least 80%, 85%, 90%, 95%, 96%, 97%, 98%, or 99% identical to SEQ ID NO: 20, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the GLA of SEQ ID NO: 21, and
SEQ ID NO: 30 or a nucleotide sequence at least 70%, 75%, 80%, 85%, 90%, 95%, 96%, 97%, 98%, or 99% identical to SEQ ID NO: 30.

### 3. Recombinant AAV, Pharmaceutical Composition and Host Cell

The present invention further provides a recombinant AAV (rAAV) comprising a genome comprising the expression construct of the invention. In some embodiments, the expression construct flanked by 5′ and 3′ inverted terminal repeats (ITRs) of AAV.

In some embodiments, the rAAV achieves a higher expression of SMN1 polypeptide and/or mRNA in spinal cord, and/or a lower expression of SMN1 polypeptide and/or mRNA in non-spinal tissues, such as heart and/or liver, as compared to a control rAAV, e.g., that comprises a nucleotide sequence of SEQ ID NO: 1.

In some embodiments, the rAAV of the invention can be selected from human serotype 1 AAV (hAAV1), hAAV2, hAAV3, hAAV4, hAAV5, hAAV6, hAAV7, hAAV8, hAAV9, hAAV10, and hAAV11. In some embodiments, the rAAV is hAAV9.

In some embodiment, the genome of the rAAV comprises an expression construct flanked by 5′ and 3′ ITRs of AAV.

In some embodiment, the expression construct comprises a nucleotide sequence encoding SMN1 that is operably linked to a promoter. In some embodiments, the SMN1 is human SMN1. In some embodiments, the hSMN1 comprises the amino acid sequence of SEQ ID NO: 21 or an allelic variant thereof.

In some embodiments, the construct comprises a nucleotide sequence selected from SEQ ID NOs: 2, 3, 4, 5, 6, 7, 8, 9, 10, 11, 12, 13, 14, 15, 16, 17, 18, 19 and 20. Surprisingly, the inventors found that the polynucleotide with a nucleotide sequence of SEQ ID NOs: 4, 5, 6, 7, 8, 11, 16 and 19 achieves a higher expression of hSMN1 (1.5-4 folds) than SEQ ID NO: 1 in cells; while in the in vivo tests, SEQ ID NOs: 3, 6, 8 and 11 achieves higher expression in spinal cord, but lower expression in heart and liver, as compared to SEQ ID NO: 1.

In some embodiments, the nucleotide sequence is set forth in SEQ ID NO: 2 or a nucleotide sequence at least 80%, 85%, 90%, 95%, 96%, 97%, 98%, or 99% identical to SEQ ID NO: 2. In some embodiments, the polynucleotide comprises a nucleotide sequence at least 80%, 85%, 90%, 95%, 96%, 97%, 98%, or 99% identical to SEQ ID NO: 2, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 2, and encodes the polypeptide of SEQ ID NO: 21 or an allelic variant thereof.

In some embodiments, the nucleotide sequence is set forth in SEQ ID NO: 3 or a nucleotide sequence at least 80%, 85%, 90%, 95%, 96%, 97%, 98%, or 99% identical to SEQ ID NO: 3. In some embodiments, the polynucleotide comprises a nucleotide sequence at least 80%, 85%, 90%, 95%, 96%, 97%, 98%, or 99% identical to SEQ ID NO: 3, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 3, and encodes the polypeptide of SEQ ID NO: 21 or an allelic variant thereof.

In some embodiments, the nucleotide sequence is set forth in SEQ ID NO: 4 or a nucleotide sequence at least 80%, 85%, 90%, 95%, 96%, 97%, 98%, or 99% identical to SEQ ID NO: 4. In some embodiments, the polynucleotide comprises a nucleotide sequence at least 80%, 85%, 90%, 95%, 96%, 97%, 98%, or 99% identical to SEQ ID NO: 4, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 4 and encodes the polypeptide of SEQ ID NO: 21 or an allelic variant thereof.

In some embodiments, the nucleotide sequence is set forth in SEQ ID NO: 5 or a nucleotide sequence at least 80%, 85%, 90%, 95%, 96%, 97%, 98%, or 99% identical to SEQ ID NO: 5. In some embodiments, the polynucleotide comprises a nucleotide sequence at least 80%, 85%, 90%, 95%, 96%, 97%, 98%, or 99% identical to SEQ ID NO: 5, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 5, and encodes the polypeptide of SEQ ID NO: 21 or an allelic variant thereof.

In some embodiments, the nucleotide sequence is set forth in SEQ ID NO: 6 or a nucleotide sequence at least 80%, 85%, 90%, 95%, 96%, 97%, 98%, or 99% identical to SEQ ID NO: 6. In some embodiments, the polynucleotide comprises a nucleotide sequence at least 80%, 85%, 90%, 95%, 96%, 97%, 98%, or 99% identical to SEQ ID NO: 6, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 6, and encodes the polypeptide of SEQ ID NO: 21 or an allelic variant thereof.

In some embodiments, the nucleotide sequence is set forth in SEQ ID NO: 7 or a nucleotide sequence at least 80%, 85%, 90%, 95%, 96%, 97%, 98%, or 99% identical to SEQ ID NO: 7. In some embodiments, the polynucleotide comprises a nucleotide sequence at least 80%, 85%, 90%, 95%, 96%, 97%, 98%, or 99% identical to SEQ ID NO: 7, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 7, and encodes the polypeptide of SEQ ID NO: 21 or an allelic variant thereof.

In some embodiments, the nucleotide sequence is set forth in SEQ ID NO: 8 or a nucleotide sequence at least 80%, 85%, 90%, 95%, 96%, 97%, 98%, or 99% identical to SEQ ID NO: 8. In some embodiments, the polynucleotide comprises a nucleotide sequence at least 80%, 85%, 90%, 95%, 96%, 97%, 98%, or 99% identical to SEQ ID NO: 8, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 8, and encodes the polypeptide of SEQ ID NO: 21 or an allelic variant thereof.

In some embodiments, the nucleotide sequence is set forth in SEQ ID NO: 9 or a nucleotide sequence at least 80%, 85%, 90%, 95%, 96%, 97%, 98%, or 99% identical to SEQ ID NO: 9. In some embodiments, the polynucleotide comprises a nucleotide sequence at least 80%, 85%, 90%, 95%, 96%, 97%, 98%, or 99% identical to SEQ ID NO: 9, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 9, and encodes the polypeptide of SEQ ID NO: 21 or an allelic variant thereof.

In some embodiments, the nucleotide sequence is set forth in SEQ ID NO: 10 or a nucleotide sequence at least 80%, 85%, 90%, 95%, 96%, 97%, 98%, or 99% identical to SEQ ID NO: 10. In some embodiments, the polynucleotide comprises a nucleotide sequence at least 80%, 85%, 90%, 95%, 96%, 97%, 98%, or 99% identical to SEQ ID NO: 10, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 10, and encodes the polypeptide of SEQ ID NO: 21 or an allelic variant thereof.

In some embodiments, the nucleotide sequence is set forth in SEQ ID NO: 11 or a nucleotide sequence at least 80%, 85%, 90%, 95%, 96%, 97%, 98%, or 99% identical to SEQ ID NO: 11. In some embodiments, the polynucleotide comprises a nucleotide sequence at least 80%, 85%, 90%, 95%, 96%, 97%, 98%, or 99% identical to SEQ ID NO: 11, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 11, and encodes the polypeptide of SEQ ID NO: 21 or an allelic variant thereof.

In some embodiments, the nucleotide sequence is set forth in SEQ ID NO: 12 or a nucleotide sequence at least 80%, 85%, 90%, 95%, 96%, 97%, 98%, or 99% identical to SEQ ID NO: 12. In some embodiments, the polynucleotide comprises a nucleotide sequence at least 80%, 85%, 90%, 95%, 96%, 97%, 98%, or 99% identical to SEQ ID NO: 12, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 12, and encodes the polypeptide of SEQ ID NO: 21 or an allelic variant thereof.

In some embodiments, the nucleotide sequence is set forth in SEQ ID NO: 13 or a nucleotide sequence at least 80%, 85%, 90%, 95%, 96%, 97%, 98%, or 99% identical to SEQ ID NO: 13. In some embodiments, the polynucleotide comprises a nucleotide sequence at least 80%, 85%, 90%, 95%, 96%, 97%, 98%, or 99% identical to SEQ ID NO: 13, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 13, and encodes the polypeptide of SEQ ID NO: 21 or an allelic variant thereof.

In some embodiments, the nucleotide sequence is set forth in SEQ ID NO: 14 or a nucleotide sequence at least 80%, 85%, 90%, 95%, 96%, 97%, 98%, or 99% identical to SEQ ID NO: 14. In some embodiments, the polynucleotide comprises a nucleotide sequence at least 80%, 85%, 90%, 95%, 96%, 97%, 98%, or 99% identical to SEQ ID NO: 14, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 14, and encodes the polypeptide of SEQ ID NO: 21 or an allelic variant thereof.

In some embodiments, the nucleotide sequence is set forth in SEQ ID NO: 15 or a nucleotide sequence at least 80%, 85%, 90%, 95%, 96%, 97%, 98%, or 99% identical to SEQ ID NO: 15. In some embodiments, the polynucleotide comprises a nucleotide sequence at least 80%, 85%, 90%, 95%, 96%, 97%, 98%, or 99% identical to SEQ ID NO: 15, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 15, and encodes the polypeptide of SEQ ID NO: 21 or an allelic variant thereof.

In some embodiments, the nucleotide sequence is set forth in SEQ ID NO: 16 or a nucleotide sequence at least 80%, 85%, 90%, 95%, 96%, 97%, 98%, or 99% identical to SEQ ID NO: 16.

In some embodiments, the polynucleotide comprises a nucleotide sequence at least 80%, 85%, 90%, 95%, 96%, 97%, 98%, or 99% identical to SEQ ID NO: 16, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 16, and encodes the polypeptide of SEQ ID NO: 21 or an allelic variant thereof.

In some embodiments, the nucleotide sequence is set forth in SEQ ID NO: 17 or a nucleotide sequence at least 80%, 85%, 90%, 95%, 96%, 97%, 98%, or 99% identical to SEQ ID NO: 17. In some embodiments, the polynucleotide comprises a nucleotide sequence at least 80%, 85%, 90%, 95%, 96%, 97%, 98%, or 99% identical to SEQ ID NO: 17, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 17, and encodes the polypeptide of SEQ ID NO: 21 or an allelic variant thereof.

In some embodiments, the nucleotide sequence is set forth in SEQ ID NO: 18 or a nucleotide sequence at least 80%, 85%, 90%, 95%, 96%, 97%, 98%, or 99% identical to SEQ ID NO: 18. In some embodiments, the polynucleotide comprises a nucleotide sequence at least 80%, 85%, 90%, 95%, 96%, 97%, 98%, or 99% identical to SEQ ID NO: 18, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 18, and encodes the polypeptide of SEQ ID NO: 21 or an allelic variant thereof.

In some embodiments, the nucleotide sequence is set forth in SEQ ID NO: 19 or a nucleotide sequence at least 80%, 85%, 90%, 95%, 96%, 97%, 98%, or 99% identical to SEQ ID NO: 19. In some embodiments, the polynucleotide comprises a nucleotide sequence at least 80%, 85%, 90%, 95%, 96%, 97%, 98%, or 99% identical to SEQ ID NO: 19, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 19, and encodes the polypeptide of SEQ ID NO: 21 or an allelic variant thereof.

In some embodiments, the nucleotide sequence is set forth in SEQ ID NO: 20 or a nucleotide sequence at least 80%, 85%, 90%, 95%, 96%, 97%, 98%, or 99% identical to SEQ ID NO: 20. In some embodiments, the polynucleotide comprises a nucleotide sequence at least 80%, 85%, 90%, 95%, 96%, 97%, 98%, or 99% identical to SEQ ID NO: 20, and encodes a functional hSMN1 polypeptide, such as a hSMN1 polypeptide having at least 10%, 20%, 30%, 40%, 50%, 60% 70%, 80%, 90%, 100% or more activity of the polypeptide of SEQ ID NO: 21. In some embodiments, the polynucleotide comprises a nucleotide sequence at least 80%, 85%, 90%, 95%, 96%, 97%, 98%, or 99% identical to SEQ ID NO: 20, and encodes the polypeptide of SEQ ID NO: 21 or an allelic variant thereof.

In some embodiments, the promoter is a constitutive promoter. In some embodiments, the promoter comprises SEQ ID NO: 23 or a nucleotide sequence at least 70%, 75%, 80%, 85%, 90%, 95%, 96%, 97%, 98%, or 99% identical to SEQ ID NO: 23. In some embodiments, the promoter comprises SEQ ID NO: 28 or a nucleotide sequence at least 70%, 75%, 80%, 85%, 90%, 95%, 96%, 97%, 98%, or 99% identical to SEQ ID NO: 28. In some embodiments, the promoter comprises SEQ ID NO: 31 or a nucleotide sequence at least 70%, 75%, 80%, 85%, 90%, 95%, 96%, 97%, 98%, or 99% identical to SEQ ID NO: 31.

In some embodiments, the expression construct further comprises an enhancer. In some embodiments, the enhancer is upstream of the promoter. In some embodiments, the enhancer is a cytomegalovirus (CMV) early enhancer. In some embodiments, the enhancer comprises SEQ ID NO: 22, or a nucleotide sequence at least 70%, 75%, 80%, 85%, 90%, 95%, 96%, 97%, 98%, or 99% identical to SEQ ID NO: 22.

It is also desirable to include an intron (e.g., an intron derived from a eukaryotic organism or an artificial intron) in the construct to increase the expression in eukaryotic cells. In some embodiments, the expression construct further comprises an intron. In some embodiments, the intron is upstream of the nucleotide sequence encoding SMN1. In some embodiments, the intron is downstream of the promoter.

In some embodiments, the intron is a PCI intron set forth in SEQ ID NO: 24. In some embodiments, the intron is chimeric intron (chicken beta-actin (CBA intron and exon)+rabbit globin intron) set forth in SEQ ID NO: 29.

In some embodiments, the intron comprises SEQ ID NO: 24 or 29, or a nucleotide sequence at least 70%, 75%, 80%, 85%, 90%, 95%, 96%, 97%, 98%, or 99% identical to SEQ ID NO: 24 or 29.

The expression construct further comprises a polyadenylation signal sequence for the processing of the transcript. In some embodiments, the construct comprises a polyadenylation signal sequence downstream of the nucleotide sequence encoding hSMN1. In some embodiments, the polyadenylation signal sequence comprises SEQ ID NO: 25 or 30, or a nucleotide sequence at least 70%, 75%, 80%, 85%, 90%, 95%, 96%, 97%, 98%, or 99% identical to SEQ ID NO: 25 or 30.

In some embodiments, the expression construct comprises, in the order of 5′ to 3′, an enhancer, a promoter, an intron, a polynucleotide of interest encoding hSMN1, and a polyadenylation signal sequence.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 2 or a nucleotide sequence at least 80%, 85%, 90%, 95%, 96%, 97%, 98%, or 99% identical to SEQ ID NO: 2, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 3 or a nucleotide sequence at least 80%, 85%, 90%, 95%, 96%, 97%, 98%, or 99% identical to SEQ ID NO: 3, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 4 or a nucleotide sequence at least 80%, 85%, 90%, 95%, 96%, 97%, 98%, or 99% identical to SEQ ID NO: 4, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 5 or a nucleotide sequence at least 80%, 85%, 90%, 95%, 96%, 97%, 98%, or 99% identical to SEQ ID NO: 5, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 6 or a nucleotide sequence at least 80%, 85%, 90%, 95%, 96%, 97%, 98%, or 99% identical to SEQ ID NO: 6, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 7 or a nucleotide sequence at least 80%, 85%, 90%, 95%, 96%, 97%, 98%, or 99% identical to SEQ ID NO: 7, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 8 or a nucleotide sequence at least 80%, 85%, 90%, 95%, 96%, 97%, 98%, or 99% identical to SEQ ID NO: 8, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 9 or a nucleotide sequence at least 80%, 85%, 90%, 95%, 96%, 97%, 98%, or 99% identical to SEQ ID NO: 9, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 10 or a nucleotide sequence at least 80%, 85%, 90%, 95%, 96%, 97%, 98%, or 99% identical to SEQ ID NO: 10, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 12 or a nucleotide sequence at least 80%, 85%, 90%, 95%, 96%, 97%, 98%, or 99% identical to SEQ ID NO: 12, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 13 or a nucleotide sequence at least 80%, 85%, 90%, 95%, 96%, 97%, 98%, or 99% identical to SEQ ID NO: 13, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 14 or a nucleotide sequence at least 80%, 85%, 90%, 95%, 96%, 97%, 98%, or 99% identical to SEQ ID NO: 14, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 15 or a nucleotide sequence at least 80%, 85%, 90%, 95%, 96%, 97%, 98%, or 99% identical to SEQ ID NO: 15, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 16 or a nucleotide sequence at least 80%, 85%, 90%, 95%, 96%, 97%, 98%, or 99% identical to SEQ ID NO: 16, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 17 or a nucleotide sequence at least 80%, 85%, 90%, 95%, 96%, 97%, 98%, or 99% identical to SEQ ID NO: 17, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 18 or a nucleotide sequence at least 80%, 85%, 90%, 95%, 96%, 97%, 98%, or 99% identical to SEQ ID NO: 18, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 19 or a nucleotide sequence at least 80%, 85%, 90%, 95%, 96%, 97%, 98%, or 99% identical to SEQ ID NO: 19, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 20 or a nucleotide sequence at least 80%, 85%, 90%, 95%, 96%, 97%, 98%, or 99% identical to SEQ ID NO: 20, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 2 or a nucleotide sequence at least 80%, 85%, 90%, 95%, 96%, 97%, 98%, or 99% identical to SEQ ID NO: 2, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 3 or a nucleotide sequence at least 80%, 85%, 90%, 95%, 96%, 97%, 98%, or 99% identical to SEQ ID NO: 3, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 4 or a nucleotide sequence at least 80%, 85%, 90%, 95%, 96%, 97%, 98%, or 99% identical to SEQ ID NO: 4, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 5 or a nucleotide sequence at least 80%, 85%, 90%, 95%, 96%, 97%, 98%, or 99% identical to SEQ ID NO: 5, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 6 or a nucleotide sequence at least 80%, 85%, 90%, 95%, 96%, 97%, 98%, or 99% identical to SEQ ID NO: 6, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 7 or a nucleotide sequence at least 80%, 85%, 90%, 95%, 96%, 97%, 98%, or 99% identical to SEQ ID NO: 7, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 8 or a nucleotide sequence at least 80%, 85%, 90%, 95%, 96%, 97%, 98%, or 99% identical to SEQ ID NO: 8, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 9 or a nucleotide sequence at least 80%, 85%, 90%, 95%, 96%, 97%, 98%, or 99% identical to SEQ ID NO: 9, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 10 or a nucleotide sequence at least 80%, 85%, 90%, 95%, 96%, 97%, 98%, or 99% identical to SEQ ID NO: 10, wherein the nucleotide sequence encodes the hSMN1 of SEQ ID) NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 12 or a nucleotide sequence at least 80%, 85%, 90%, 95%, 96%, 97%, 98%, or 99% identical to SEQ ID NO: 12, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 13 or a nucleotide sequence at least 80%, 85%, 90%, 95%, 96%, 97%, 98%, or 99% identical to SEQ ID NO: 13, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 14 or a nucleotide sequence at least 80%, 85%, 90%, 95%, 96%, 97%, 98%, or 99% identical to SEQ ID NO: 14, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 15 or a nucleotide sequence at least 80%, 85%, 90%, 95%, 96%, 97%, 98%, or 99% identical to SEQ ID NO: 15, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 16 or a nucleotide sequence at least 80%, 85%, 90%, 95%, 96%, 97%, 98%, or 99% identical to SEQ ID NO: 16, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 17 or a nucleotide sequence at least 80%, 85%, 90%, 95%, 96%, 97%, 98%, or 99% identical to SEQ ID NO: 17, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 18 or a nucleotide sequence at least 80%, 85%, 90%, 95%, 96%, 97%, 98%, or 99% identical to SEQ ID NO: 18, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 19 or a nucleotide sequence at least 80%, 85%, 90%, 95%, 96%, 97%, 98%, or 99% identical to SEQ ID NO: 19, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 22 or a nucleotide sequence at least 70%, 75%, 80%, 85%, 90%, 95%, 96%, 97%, 98%, or 99% identical to SEQ ID NO: 22,
SEQ ID NO: 23 or a nucleotide sequence at least 70%, 75%, 80%, 85%, 90%, 95%, 96%, 97%, 98%, or 99% identical to SEQ ID NO: 23,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 20 or a nucleotide sequence at least 80%, 85%, 90%, 95%, 96%, 97%, 98%, or 99% identical to SEQ ID NO: 20, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 28 or a nucleotide sequence at least 70%, 75%, 80%, 85%, 90%, 95%, 96%, 97%, 98%, or 99% identical to SEQ ID NO: 28,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 25 or a nucleotide sequence at least 70%, 75%, 80%, 85%, 90%, 95%, 96%, 97%, 98%, or 99% identical to SEQ ID NO: 25.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 24 or a nucleotide sequence at least 70%, 75%, 80%, 85%, 90%, 95%, 96%, 97%, 98%, or 99% identical to SEQ ID NO: 24,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the hSMN1 of SEQ ID NO: 21, and
SEQ ID NO: 30 or a nucleotide sequence at least 70%, 75%, 80%, 85%, 90%, 95%, 96%, 97%, 98%, or 99% identical to SEQ ID NO: 30.

In some embodiments, the expression construct comprises, in the order of 5′ to 3′,
SEQ ID NO: 31 or a nucleotide sequence at least 70%, 75%, 80%, 85%, 90%, 95%, 96%, 97%, 98%, or 99% identical to SEQ ID NO: 31,
SEQ ID NO: 29 or a nucleotide sequence at least 70%, 75%, 80%, 85%, 90%, 95%, 96%, 97%, 98%, or 99% identical to SEQ ID NO: 29,
SEQ ID NO: 11 or a nucleotide sequence at least 80%, 85%, 90%, 95%, 96%, 97%, 98%, or 99% identical to SEQ ID NO: 11, wherein the nucleotide sequence encodes the hSMN1 of SEQ ID NO: 21 or an allelic variant thereof, or a functional hSMN1 polypeptide, such as a hSMN1 polypeptide having at least 10%, 20%, 30%, 40%, 50%, 60% 70%, 80%, 90%, 100% or more activity of the GLA of SEQ ID NO: 21, and

SEQ ID NO: 30 or a nucleotide sequence at least 70%, 75%, 80%, 85%, 90%, 95%, 96%, 97%, 98%, or 99% identical to SEQ ID NO: 30.

The present invention also provides a method for preparing the rAAV. In some embodiments, the rAAV is prepared by a system containing a transgene plasmid comprising the genome of the rAAV, a packaging plasmid encoding the REP and/or CAP proteins, and a helper plasmid, e.g., a host cell such as a mammalian cell comprising the transgene plasmid comprising the genome of the rAAV, the packaging plasmid encoding the REP and/or CAP proteins, and the helper plasmid. Therefore, the present invention also provides a vector such as a plasmid comprising the genome of the rAAV of the invention.

In some embodiments, the rAAV can be packaged as described in Crosson S M et al. (Crosson S M, Dib P, Smith J K, Zolotukhin S. Helper-free Production of Laboratory Grade AAV and Purification by Iodixanol Density Gradient Centrifugation. Mol Ther Methods Clin Dev. 2018; 10:1-7).

The present invention further provides a vector comprising the genome of the rAAV, wherein the genome of rAAV comprises the expression construct of the invention flanked by 5′ and 3′ ITRs. In some embodiments, the vector is a transgene plasmid for the packaging of rAAV.

In some embodiments, the genome of the rAAV is a self-complementary vector genome (scAAV).

In some embodiments, the genome of the rAAV comprises, in the order of 5′ to 3′. 5′ ITR, SEQ ID NO: 31, SEQ ID NO: 24, SEQ ID NO: 11, SEQ ID NO: 25, and 3′ ITR. In some embodiments, the genome of the rAAV comprises, in the order of 5′ to 3′, 5′ ITR, SEQ ID NO: 31, SEQ ID NO: 29, SEQ ID NO: 11, SEQ ID NO: 25, and 3′ ITR. In some embodiments, the genome of the rAAV comprises, in the order of 5′ to 3′, 5′ ITR, SEQ ID NO: 31, SEQ ID NO: 24, SEQ ID NO: 11, SEQ ID NO: 30, and 3′ ITR. In some embodiments, the genome of the rAAV comprises, in the order of 5′ to 3′, 5′ ITR, SEQ ID NO: 31, SEQ ID NO: 29, SEQ ID NO: 11, SEQ ID NO: 30, and 3′ ITR.

In some embodiments, the genome of the rAAV comprises, in the order of 5′ to 3′, 5′ ITR, SEQ ID NO: 28, SEQ ID NO: 24, SEQ ID NO: 11, SEQ ID NO: 25, and 3′ ITR. In some embodiments, the genome of the rAAV comprises, in the order of 5′ to 3′, 5′ ITR, SEQ ID NO: 28, SEQ ID NO: 29, SEQ ID NO: 11, SEQ ID NO: 25, and 3′ ITR. In some embodiments, the genome of the rAAV comprises, in the order of 5′ to 3′, 5′ ITR, SEQ ID NO: 28, SEQ ID NO: 24, SEQ ID NO: 11, SEQ ID NO: 30, and 3′ ITR. In some embodiments, the genome of the rAAV comprises, in the order of 5′ to 3′, 5′ ITR, SEQ ID NO: 28, SEQ ID NO: 29, SEQ ID NO: 11, SEQ ID NO: 30, and 3′ ITR.

In some embodiments, the 5′ITR comprises SEQ ID NO: 26. In some embodiments, the 3′ ITR comprises SEQ ID NO: 27.

In some embodiments, the genome of the rAAV comprises, in the order of 5′ to 3′, SEQ ID NO: 26, SEQ ID NO: 31, SEQ ID NO: 24, SEQ ID NO: 11, SEQ ID NO: 25, and SEQ ID NO: 27. In some embodiments, the genome of the rAAV comprises, in the order of 5′ to 3′, SEQ ID NO: 26, SEQ ID NO: 31, SEQ ID NO: 29, SEQ ID NO: 11, SEQ ID NO: 25, and SEQ ID NO: 27. In some embodiments, the genome of the rAAV comprises, in the order of 5′ to 3′, SEQ ID NO: 26, SEQ ID NO: 31, SEQ ID NO: 24, SEQ ID NO: 11, SEQ ID NO: 30, and SEQ ID NO: 27. In some embodiments, the genome of the rAAV comprises, in the order of 5′ to 3′, SEQ ID NO: 26, SEQ ID NO: 31, SEQ ID NO: 29, SEQ ID NO: 11, SEQ ID NO: 30, and SEQ ID NO: 27.

In some embodiments, the genome of the rAAV comprises, in the order of 5′ to 3′, SEQ ID NO: 26, SEQ ID NO: 28, SEQ ID NO: 24, SEQ ID NO: 11, SEQ ID NO: 25, and SEQ ID NO: 27. In some embodiments, the genome of the rAAV comprises, in the order of 5′ to 3′, SEQ ID NO: 26, SEQ ID NO: 28, SEQ ID NO: 29, SEQ ID NO: 11, SEQ ID NO: 25, and SEQ ID NO: 27. In some embodiments, the genome of the rAAV comprises, in the order of 5′ to 3′, SEQ ID NO: 26, SEQ ID NO: 28, SEQ ID NO: 24, SEQ ID NO: 11, SEQ ID NO: 30, and SEQ ID NO: 27. In some embodiments, the genome of the rAAV comprises, in the order of 5′ to 3′, SEQ ID NO: 26, SEQ ID NO: 28, SEQ ID NO: 29, SEQ ID NO: 11, SEQ ID NO: 30, and SEQ ID NO: 27.

The present invention also provides a pharmaceutical composition comprising the rAAV. Pharmaceutical compositions containing the AAV of the invention can be formulated in any conventional manner by mixing a selected amount of the rAAV with one or more pharmaceutically acceptable carriers or excipients.

Selection of the carrier or excipient is within the skill of the administering professional and can depend upon a number of parameters. These include, for example, the mode of administration (i.e., systemic, oral, local, topical or any other mode) and the disease to be treated. Pharmaceutical carriers or vehicles suitable for administration of the rAAV provided herein include any such carriers known to those skilled in the art to be suitable for the particular mode of administration.

In some embodiments, the pharmaceutical composition is formulated for parenteral administration, e.g., intravenous, intramuscular, or subcutaneous injection. In some embodiments, the pharmaceutical composition is formulated for local administration.

The inventors found that the pharmaceutical composition of the present invention can achieve a desired safety, e.g., when being administered by intravenous injection. For example, the safety can be evaluated by the expression level of hSMN1 in tissues other than spinal cord, such as liver and heart.

The present invention further provides a host cell comprising the polynucleotide, the expression construct, and/or the vector of the present invention. In some embodiment, the host cell is a mammalian cell, such as a human cell. In some embodiment, the host cell is HEK293T cell.

### 4. Treatment of Diseases

The present invention provides a method of treating a disease associated with the deficiency of SMN1, comprising administering the rAAV or the pharmaceutical composition of the present invention to a subject in need thereof. In some embodiments, the disease is SMA, in particular, Type I SMA. In some embodiments, the rAAV or the pharmaceutical composition is administered by intravenous injection.

The present invention further provides use of the polynucleotide, the expression construct, the rAAV, the pharmaceutical composition, the vector or the host cell of the present invention in the preparation of a medicament for treating a disease associated with the deficiency of SMN1 in a subject in need thereof. In some embodiments, the disease is SMA, in particular, Type I SMA. In some embodiments, the medicament is administered by intravenous injection.

Provided is the rAAV and/or the pharmaceutical composition of the present invention, for use in the treatment of a disease associated with the deficiency of SMN1 in a subject in need thereof. In some embodiments, the disease is SMA, in particular, Type I SMA. In some embodiments, the rAAV and/or the pharmaceutical composition is administered by intravenous injection.

In some embodiments, the treatment increases the hSMN1 activity in the subject. In some embodiments, the treatment increases the hSMN1 activity in spinal cord. In some embodiments, the treatment reduces the death of neurons in anterior horn of spinal cord, thereby alleviating atrophy. In some embodiments, the subject is a human.

In some embodiments, the treatment cures, improves, alleviates, blocks or partially blocks the symptoms of SMA.

### Embodiments

Clause 1. A polynucleotide comprising a nucleotide sequence encoding a SMN1, preferably human SMN1 (hSMN1) polypeptide, wherein the nucleotide sequence is codon-optimized to improve the expression.

Clause 2. The polynucleotide of Clause 1, wherein the nucleotide sequence is set forth in SEQ ID NOs: 2, 3, 4, 5, 6, 7, 8, 9, 10, 11, 12, 13, 14, 15, 16, 17, 18, 19 or 20, a variant thereof.

Clause 3. The polynucleotide of Clause 2, wherein the variant is at least 80%, 85%, 90%, 95%, 96%, 97%, 98%, or 99% identical to SEQ ID NOs: 2, 3, 4, 5, 6, 7, 8, 9, 10, 11, 12, 13, 14, 15, 16, 17, 18, 19 or 20.

Clause 4. The polynucleotide of any of Clauses 1-3, wherein the SMN1 polypeptide is a hSMN1 polypeptide having at least 10%, 20%, 30%, 40%, 50%, 60% 70%, 80%, 90%, 100% or more activity of the polypeptide of SEQ ID NO: 21.

Clause 5. The polynucleotide of any of Clauses 1-4, wherein the SMN1 polypeptide comprises SEQ ID NO: 21 or an allelic variant thereof.

Clause 6. The polynucleotide of any of Clauses 1-5, the nucleotide sequence is set forth in SEQ ID NOs: 3, 4, 5, 6, 7, 8, 11, 16 or 19.

Clause 7. The polynucleotide of any of Clauses 1-6, the nucleotide sequence is set forth in SEQ ID NOs: 3, 6, 8, or 11.

Clause 8. The polynucleotide of any of Clauses 1-7, the nucleotide sequence is set forth in SEQ ID NO: 11.

Clause 9. An expression construct comprising the polynucleotide of any of Clause 1-8 operably linked to a promoter.

Clause 10. The expression construct of Clause 9, wherein the promoter comprises a nucleotide sequence of SEQ ID NO: 23, 28 or 31, or a nucleotide sequence at least 70%, 75%, 80%, 85%, 90%, 95%, 96%, 97%, 98%, or 99% identical to SEQ ID NO: 23, 28 or 31.

Clause 11. The expression construct of Clause 9 or 10, wherein the promoter comprises a nucleotide sequence of SEQ ID NO: 31.

Clause 12. The expression construct of any of Clauses 9-11, wherein the construct further comprises an enhancer, preferably upstream of the promoter.

Clause 13. The expression construct of Clause 12, wherein the enhancer comprises a nucleotide sequence of SEQ ID NO: 22, or a nucleotide sequence at least 70%, 75%, 80%, 85%, 90%, 95%, 96%, 97%, 98%, or 99% identical to SEQ ID NO: 22.

Clause 14. The expression construct of any of Clauses 9-13, wherein the construct further comprises an intron, preferably downstream of the promoter and upstream of the nucleotide sequence encoding the SMN1 polypeptide.

Clause 15. The expression construct of Clause 14, wherein the intron comprises a nucleotide sequence of SEQ ID NO: 24 or 29, or a nucleotide sequence at least 70%, 75%, 80%, 85%, 90%, 95%, 96%, 97%, 98%, or 99% identical to SEQ ID NO: 24 or 29.

Clause 16. The expression construct of any of Clauses 9-15, wherein the construct further comprises a polyadenylation signal sequence downstream of the nucleotide sequence encoding the SMN1 polypeptide.

Clause 17. The expression construct of Clause 16, wherein the polyadenylation signal sequence comprises a nucleotide sequence of SEQ ID NO: 25 or 30, or a nucleotide sequence at least 70%, 75%, 80%, 85%, 90%, 95%, 96%, 97%, 98%, or 99% identical to SEQ ID NO: 25 or 30.

Clause 18. The expression construct of any of Clauses 9-17, wherein the construct comprises in the order of 5′ to 3′, an enhancer, a promoter, an intron, a polynucleotide of interest encoding hSMN1, and a polyadenylation signal sequence.

Clause 19. The expression construct of any of Clauses 9-18, wherein the construct comprises in the order of 5′ to 3′,
i) SEQ ID NO: 31, SEQ ID NO: 24, SEQ ID NO: 11, SEQ ID NO: 25;
ii) SEQ ID NO: 31, SEQ ID NO: 29, SEQ ID NO: 11, SEQ ID NO: 25;
iii) SEQ ID NO: 31, SEQ ID NO: 24, SEQ ID NO: 11, SEQ ID NO: 30;
iv) SEQ ID NO: 31, SEQ ID NO: 29, SEQ ID NO: 11, SEQ ID NO: 30;
v) SEQ ID NO: 28, SEQ ID NO: 24, SEQ ID NO: 11, SEQ ID NO: 25;
vi) SEQ ID NO: 28, SEQ ID NO: 29, SEQ ID NO: 11, SEQ ID NO: 25;
vii) SEQ ID NO: 28, SEQ ID NO: 24, SEQ ID NO: 11, SEQ ID NO: 30; or
viii) SEQ ID NO: 28, SEQ ID NO: 29, SEQ ID NO: 11, SEQ ID NO: 30.

Clause 20. A recombinant adeno-associated virus (rAAV) comprising a genome comprising the expression construct of any of Clause 9-19 flanked by 5′ and 3′ ITRs.

Clause 21. The rAAV of Clause 20, wherein the rAAV is selected from human serotype 1 AAV (hAAV1), hAAV2, hAAV3, hAAV4, hAAV5, hAAV6, hAAV7, hAAV8, hAAV9, hAAV10, and hAAV11, preferably, the rAAV is hAAV9.

Clause 22. The rAAV of Clause 20 or 21, wherein the genome of the rAAV, from 5′ end of the 5′ ITR to the 3′ end of the 3′ ITR, is about 4,700 nucleotides in length, such as 4,600-4,800, 4,620-4,780, 4,640-4,760, 4,650-4,750, 4,660-4,740, 4,670-4,730, 4,680-4,720 or 4,690-4,710 nucleotides.

Clause 23. The rAAV of any of Clauses 20-22, wherein the genome of the rAAV comprises a stuffer fragment, preferably downstream of the polyadenylation signal sequence.

Clause 24. The rAAV of any of Clauses 20-23, wherein the 5′ ITR comprises SEQ ID NO: 26.

Clause 25. The rAAV of any of Clauses 20-24, wherein the 3′ ITR comprises SEQ ID NO: 27.

Clause 26. The rAAV of any of Clauses 20-25, wherein the genome of the rAAV comprises, in the order of 5′ to 3′,
i) SEQ ID NO: 26, SEQ ID NO: 31, SEQ ID NO: 24, SEQ ID NO: 11, SEQ ID NO: 25, SEQ ID NO: 27;
ii) SEQ ID NO: 26, SEQ ID NO: 31, SEQ ID NO: 29, SEQ ID NO: 11, SEQ ID NO: 25, SEQ ID NO: 27;
iii) SEQ ID NO: 26, SEQ ID NO: 31, SEQ ID NO: 24, SEQ ID NO: 11, SEQ ID NO: 30, SEQ ID NO: 27
v) SEQ ID NO: 26, SEQ ID NO: 28, SEQ ID NO: 24, SEQ ID NO: 11, SEQ ID NO: 25, SEQ ID NO: 27;
vi) SEQ ID NO: 26, SEQ ID NO: 28, SEQ ID NO: 29, SEQ ID NO: 11, SEQ ID NO: 25, SEQ ID NO: 27;
vii) SEQ ID NO: 26, SEQ ID NO: 28, SEQ ID NO: 24, SEQ ID NO: 11, SEQ ID NO: 30, SEQ ID NO: 27; or
viii) SEQ ID NO: 26, SEQ ID NO: 28, SEQ ID NO: 29, SEQ ID NO: 11, SEQ ID NO: 30, SEQ ID NO: 27.

Clause 27. A vector comprising the genome of the rAAV of any of Clause 20-26.

Clause 28. The vector of Clause 27, wherein is a transgene plasmid for the packaging of the rAAV.

Clause 29. A host cell comprising the polynucleotide of any of Clauses 1-8, the expression construct of Clauses 9-19, the rAAV of any of Clauses 20-26, or the vector of Clause 27 or 28.

Clause 30. The host cell of Clause 29, wherein the host cell is a mammalian cell, such as a human cell.

Clause 31. The host cell of Clause 29 or 30, wherein the host cell is HEK293T cell.

Clause 32. A pharmaceutical composition comprising the rAAV of any of Clauses 20-26.

Clause 33. The pharmaceutical composition of Clause 32, further comprising a pharmaceutically acceptable carrier or excipient.

Clause 34. The pharmaceutical composition of Clause 32 or 33, wherein the pharmaceutical composition is formulated for parenteral administration, e.g., intravenous, intramuscular, or subcutaneous injection.

Clause 35. The pharmaceutical composition of any of Clauses 32-34, wherein the pharmaceutical composition is formulated for local administration.

Clause 36. The rAAV of any of Clauses 20-26 or the pharmaceutical composition of any of Clauses 32-35, for use in treating a disease associated with the deficiency of SMN1 in a subject in need thereof.

Clause 37. A method of treating a disease associated with the deficiency of SMN1, comprising administering the rAAV of any of Clauses 20-26 or the pharmaceutical composition of any of Clauses 32-35 to a subject in need thereof.

Clause 38. Use of the polynucleotide of any of Clauses 1-8, the expression construct of Clauses 9-19, the rAAV of any of Clauses 20-26, the vector of Clause 27 or 28, the host cell of any of Clause 29-31, or the pharmaceutical composition of any of Clauses 32-35 in the preparation of a medicament for treating a disease associated with the deficiency of SMN1 in a subject in need thereof.

Clause 39. The rAAV or pharmaceutical composition for use of Clause 36, the method of Clause 37 or the use of Clause 38, wherein the disease is SMA, in particular, Type I SMA.

Clause 40. The rAAV or pharmaceutical composition for use of Clause 36, the method of Clause 37 or the use of Clause 38, wherein the subject is human.

Clause 41. The polynucleotide of any of Clauses 1-9, the expression construct of any of Clauses 10-19, or the rAAV of any of Clauses 20-26, which achieves a higher expression of SMN1 polypeptide and/or mRNA in spinal cord, and/or a lower expression of SMN1 polypeptide and/or mRNA in non-spinal tissues, such as heart and/or liver, as compared to a polynucleotide, an expression construct or an rAAV that comprises a nucleotide sequence of SEQ ID NO: 1.

### EXAMPLES

The following Examples are provided for illustration only, rather than for any limitation to the present application.

### Example 1. Expression of hSMN1 in 293T Cells

### 1.1. The Construction of Transgene Plasmid

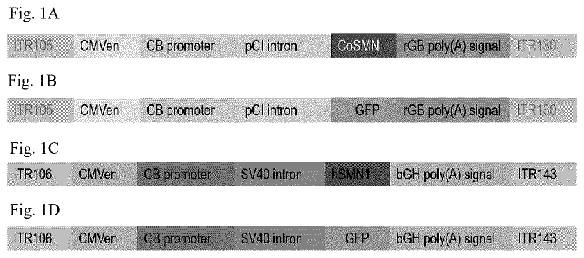

20 constructs comprising the genomes of rAAVs comprising a codon-optimized coding sequence for hSMN1 (CoSMN) or a coding sequence for GFP were prepared by Gibson Assembling the pGCB108 plasmid comprising the cytomegalovirus early (CMVen) enhancer (SEQ ID NO: 22) and the chicken beta-actin core (CB) promoter (SEQ ID NO: 23) with the PCI intron (SEQ ID NO: 24) and coding sequences (SEQ ID NOs: 2-20 or 36). The Zolgensma construct (see WO 2020/113034) was introduced into pGCB108 plasmid by Gibson assembling. The structures of the constructs are shown in FIG. 1. The transgene plasmids were referred to as Plasmids 2-20 (comprising SEQ ID NOs: 2-20, respectively), Plasmid Zol (comprising the Zolgensma construct), and Plasmid GFP (comprising the GFP coding sequence).

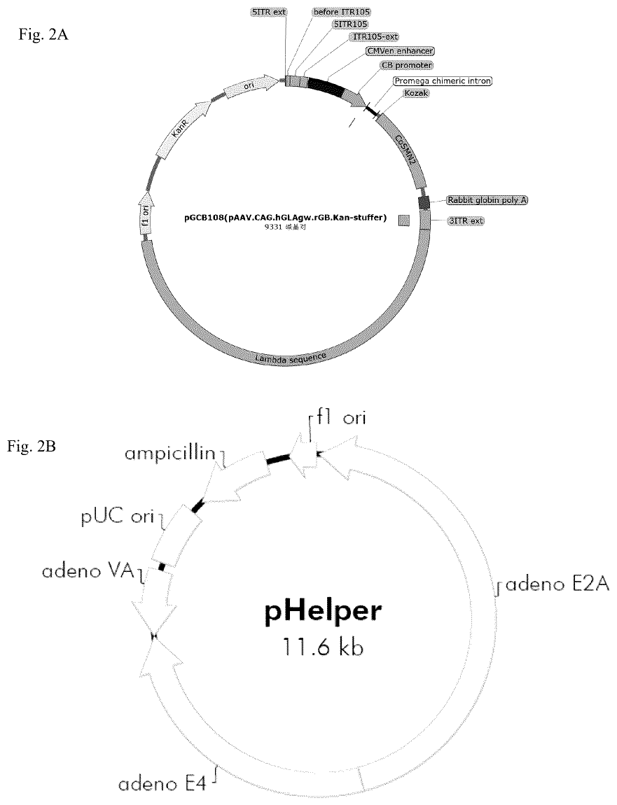

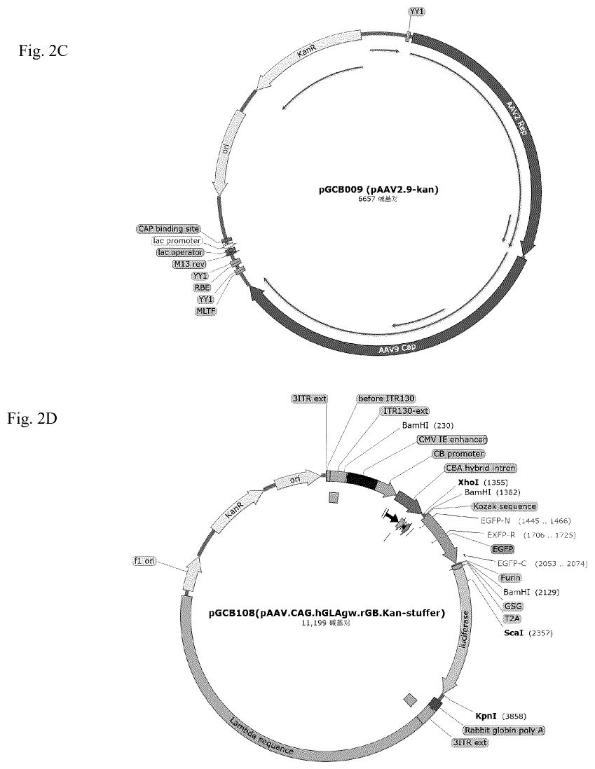

As an example, the map of the transgene plasmid comprising the optimized coding sequence for hSMN1 of SEQ ID NO: 2 is shown in FIG. 2A, and the sequence thereof is shown in SEQ ID NO: 37.

### 1.2. Transfection of 293T Cells

The 293T cells (ATCC CRL-3216™), were cultured according to manufactory's protocol at 37° C. in 5% CO₂. 293T cells were seeded in 12-well tissue culture plates at the density of 5×10⁵ cells/well one day before the transfection, the transfection was carried out with the transgene plasmids constructed in Example 1.1, respectively, according to manufactory's protocol (FuGENE® HD Transfection Reagent from Promega). Cell pellets were collected 72 h after transfection. One tenth amount of fLuc2 plasmid (see FIG. 2D) was added as the transfection efficiency control when performing transfection.

### 1.3. Western Blotting

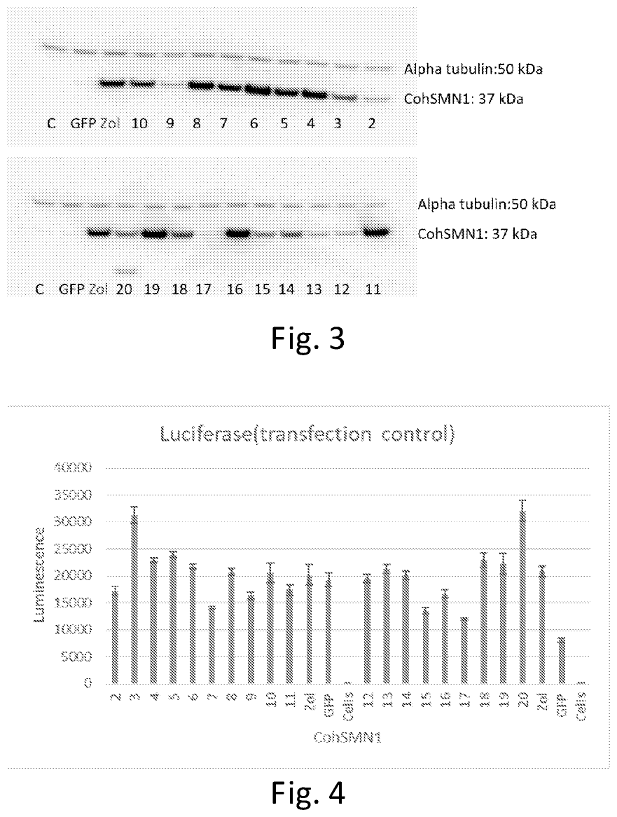

The infected cells were lysed by M-PER™ Mammalian Protein Extraction Reagent (Thermo Fisher, Catalog number: 78501). Equal amounts of the lysates (10 μg) were loaded on a NuPAGE 4-12% Bis-Tris Gel for PAGE (1 μl reducing+9 μl 4Xli-cor protein loading+30 μl sup, 200V, 22 min). The proteins were transferred onto a nitrocellulose membrane using iBlot® 2 Gel Transfer Device (Thermo Fisher Scientific) and iBlot® 2 Transfer Stack (nitrocellulose, regular size) according to the manufacturer's instructions. After one hour incubation with a blocking buffer (8% milk in 1XTBST), and rinse for 3 times (1 min/rinse), the membranes were probed with primary antibodies (SMN1 Monoclonal Antibody (2F1), Thermo MA5-15857, and Thermo alpha Tubulin Polyclonal Antibody, PA1-38814) 1:2000, 4° C., overnight in the same buffer, followed by rinse for 3 times (1 min/rinse), and then, the incubation with Donkey Anti-mouse HRP (Thermo Fisher, Cat #A16011), (1:5000), for 2 h, RT. Immunoreaction was visualized using Biorad ChemiDoc Imaging Systems. The image of the western blot for hSMN1 is shown in FIG. 3. Quantifications were performed on scanned images using the ImageJ software.

### 1.4. Luciferase Assays

Steady-Glo® Luciferase Assay System from Promega was used to detect fLuc2 activity. Equal amount of the transfected cell lysates (10 μg) 10 μl were diluted in 90 μl of OptiMEM medium, and 100 μl of the Steady-Glo® Luciferase substrate was added to black 96 well plate (Corning™ 3991, Corning™ 96-Well, Flat-Bottom Polystyrene NBS Microplate Without Lid). The plate was shaked at R/T for at least 5 minutes for mixing, then detected for luminescence in softmax luminometer (Molecular Devices SpectraMax i3x Multi-Mode Detection Platform, Manufacturer: Molecular Devices, Model: SpectraMax I3x). The luminescence of each of transfected cell samples is shown in FIG. 4.

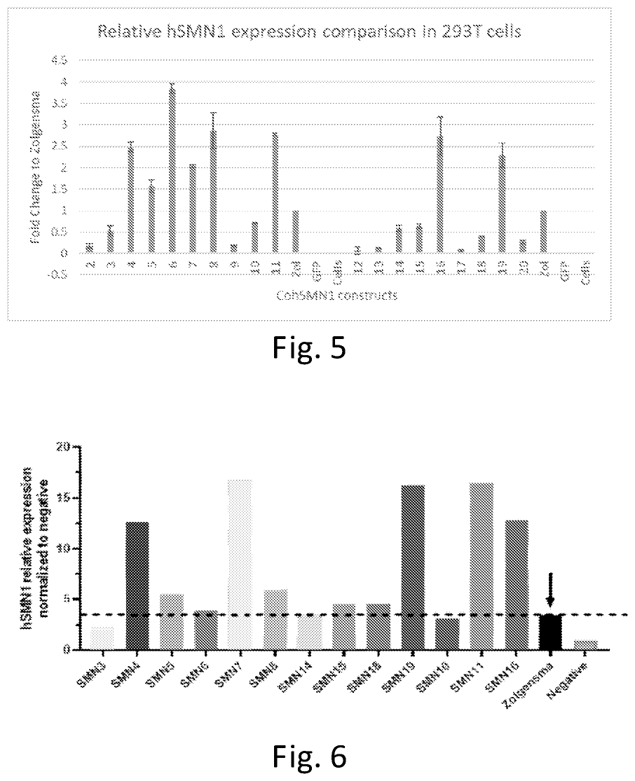

The western blot data were normalized according to the luciferase activity, and the normalized data were shown in FIG. 5. Eight of the codon-optimized sequences (CoSMN4, CoSMN5, CoSMN6, CoSMN7, CoSMN8, CoSMN11, CoSMN16 and CoSMN19) showed an expression of hSMN1 1.5-4 folds higher than Zolgensma, while 6 of the codon-optimized sequences (CoSMN2, CoSMN 9, CoSMN 12, CoSMN 13, CoSMN 17 and CoSMN 20) showed a relatively poor expression of hSMN1.

### Example 2. Preparation of rAAVs

rAAV vectors were prepared with a method similar to that described in Crosson S M et al. 2018. Briefly, 3E6 cells/ml 293VPC cells (Thermo, Catalog A35347) in serum free virus production medium OPM-293 CD05 (Shanghai OPM Biosciences Co.Ltd. Catalog: 81075-001) were triple transfected, using polyethylenimine, with the helper plasmid, the packaging plasmid encoding rep/cap, and the transgene plasmid below:
helper plasmid (“pHelper” containing the Ad E2A, E4, and VA RNA helper genes as described in Crosson Sm et al., the map thereof is shown in FIG. 2B);
packaging plasmid, the map thereof is shown in FIG. 2C, in which “AAV9 Cap” is the nucleotide sequence encoding the Cap polypeptide of AAV9 (SEQ ID NO: 34), and “AAV2 Rep” is the nucleotide sequence encoding the Rep polypeptide of AAV2 (SEQ ID NO: 35);
transgene plasmid, Plasmids 3, 4, 5, 6, 7, 8, 10, 11, 14, 15, 16, 18, and 19, as well as the Plasmid Zol as constructed in Example 1.1.

Following 72 h incubation at 37° C., cells were harvested, and viral particles were purified through an iodixanol gradient (see Crosson S M et al.).

The resulted rAAVs were referred to as SMN3, SMN4, SMN5, SMN6, SMN7, SMN8, SMN10, SMN11, SMN14, SMN15. SMN16, SMN18, SMN19, as well as the Zolgensma, and were tested for titers by ddPCR, which were all at the level of 1013 viral genomes (vg)/mL. In particular, the ddPCR was carried out with Bio-Rad's QXDx AutoDG ddPCR System and QXDx Universal Kit for AutoDG ddPCR System according to the manufacturer's instructions.

The primers and probe for the ddPCR are as follows:

| | Forward primer: |
|---|---|
| (SEQ ID NO: 38) | |
| | CCCACTTGGCAGTACATCAA |
| | Reverse primer: |
| (SEQ ID NO: 39) | |
| | GCCAAGTAGGAAAGTCCCATAA |
| | Probe: |
| (SEQ ID NO: 40) | |
| | CATAATGCCAGGCGGGCCATTTAC. |

### Example 3. Expression of hSMN1 by the rAAVs

This Example was carried out to verify the expression of hSMN1 contained in the rAAVs as prepared in Example 2, and the results showed an increased expression of the hSMN1 in the rAAVs comprising the codon-optimized coding sequence for hSMN1 of the present invention.

The rAAVs as prepared in Example 2 were infected into HEK293T cells with the MOI of 1E6 for 48 hours at 37° C.

The infected cells were lysed by M-PER™ Mammalian Protein Extraction Reagent (Thermo Fisher, Catalog number: 78501). Equal amounts of lysates (10 μg) were loaded on a SDS-PAGE Gel, and run at 200V for 22 min. The proteins were transferred onto a PVDF membrane using iBlot® 2 Gel Transfer Device (Thermo Fisher Scientific) and iBlot® 2 Transfer Stack (PVDF, regular size) according to the manufacturer's instructions. After one hour incubation with a blocking buffer (8% milk in 1XTBST), and rinse for 3 times (1 min/rinse), the membranes were probed with primary antibodies (Invitrogen MAS-15857 (2B1) SMN1 antibody (1:2,000)), 4° C., overnight in the same buffer, followed by rinse for 3 times (1 min/rinse), and then, the incubation with IRDye® 800CW Donkey anti-Mouse IgG (Licor; P/N: 926-32212), (1:10,000), for 2 h, RT. β-Actin serves as housekeeping control for quantitation of the hSMN1 relative expression. Immunoreaction was visualized using Licor Odyssey® DLx Imaging System. Image acquisition, organization, and analysis were performed on scanned images using the Empiria Studio® Software paired with Odyssey DLx Imager.

As shown in FIG. 6, the codon optimization indeed worked and rAAVs SMN4, 7, 11, 16 and 19 had 3× to 5× higher relative expressions than Zolgensma in the in vitro infection study.

### Example 4. In Vivo Expression of hSVIN1 by the rAAVs in WT Mice

This Example was carried out to evaluate the in vivo expression of hSMN1 by the rAAVs as prepared in Example 2.

The rAAVs SMN3, SMN5, SMN6, SMN8, SMN10, SMN11, SMN14, SMN15, SMN16, SMN18, SMN19 and Zolgensma were injected to FVB rice at post neonatal day (PND) 1-2 (https://www.jax.org/strain/005025) with a single IV dose of 5e10 vg/mouse (in PBS, n=5). The heart, liver, and spinal cord tissues were collected at day 28 post-injection for the evaluation of hSMN1 protein levels.

The tissues were lysed with T-PER™ Tissue Protein Extraction Reagent (Thermo, Catalog number: 78510), 100 μl T-per with proteinase inhibitor (Pierce™ Protease Inhibitor Mini Tablets, EDTA-free, Catalog number: A32955) was added to the frozen tissue, then follow the instructions of Catalog number: 78510. 50 μg of tissue lysates were loaded for the SDS-PAGE and subsequent Western Blot analysis as described in Example 3.

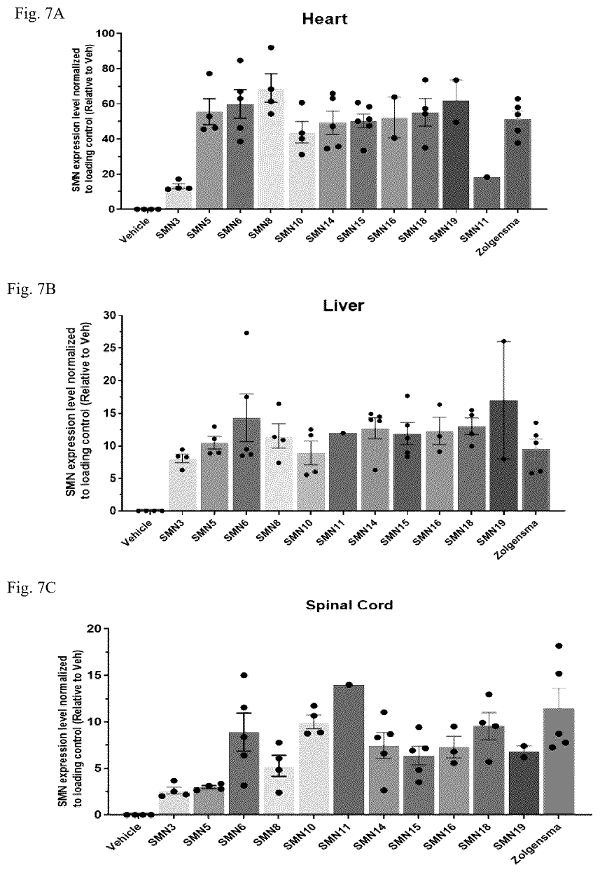

As shown in FIG. 7, the rAAVs SMN3 and SMN11 showed the lowest expression in the heart (FIG. 7A); the hSMN1 expressions of the rAAVs comprising the codon-optimized constructs in liver were similar to Zolgensma (FIG. 7B); and the rAAV SMN11 showed the highest expression in spinal cord (FIG. 7C).

### Example 5. In Vivo Expression of hSMN1 by the rAAVs in a SMA Model

This Example was carried out to investigate the expression level of hSMN1 by rAAVs in the SMNΔ7 mouse model, which is a widely used SMA disease model.

The neonatal SMNΔ7 mice (https://www.jax.org/strain/005025) were injected with rAAVs SMN6, SMN8 and SMN11 as well as the Zolgensma construct at a single IV dose of 5e10 vg/mouse (in PBS) at PND1-2 (n=7) 14 days after the injection, the heart, liver, and spinal cord tissues were collected from the injected mice to assess hSMN1 protein levels by Western Blot as described in Example 4.

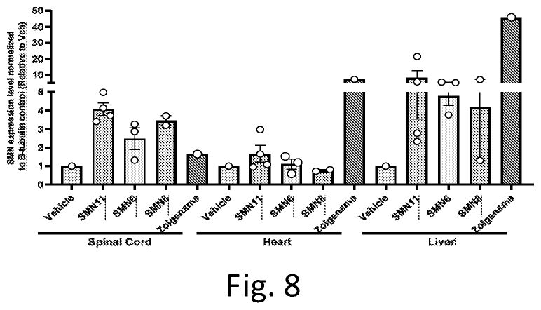

As shown in FIG. 8, the rAAVs SMN6, SMN8 and SMN11 achieved a higher hSMN1 expression in the spinal cord and significantly lower hSMN1 expressions in both liver and heart than Zolgensma, and the rAAV SMN11 achieved the highest hSMN1 expression in the spinal cord. It is known that over-expression of SMN1 in the liver and heart may cause undesired effect, hence, SMN11 was nominated as the best codon-optimized hSMN1 construct for further development.

### Example 6. Optimization of Other Elements in the rAAV Genome

This Example was carried out to achieve an improved rAAV for the treatment of SMA based on rAAV SMN11.

### 6.1. Construction of Modified Transgene Plasmids

The modified transgene plasmids were constructed by replacing elements, such as promoter, intron and polyA signal, in Plasmid 11 by Gibson Assembling. The elements in Plasmid 11 and the modified transgene plasmids were shown in Table 1.

TABLE 1
The modified transgene plasmids

| Transgene | | | | Coding | PolyA |
|---|---|---|---|---|---|
| Plasmid | Promoter | Enhancer | Intron | Sequence | Signal |
| Plasmid | SEQ ID NO: | SEQ ID | SEQ ID NO: | SEQ ID | SEQ ID |
| 11 | 23 | NO: 22 | 24 | NO: 11 | NO: 25 |
| | | | (intron1) | | |
| Plasmid | SEQ ID NO: | NA | SEQ ID NO: | SEQ ID | SEQ ID |
| 11-1 | 28 | | 24 | | |
| | (promoter 1) | | (intron1) | NO: 11 | NO: 30 |
| Plasmid | SEQ ID NO: | NA | SEQ ID NO: | SEQ ID | SEQ ID |
| 11-2 | 28 | | 29 | | |
| | (promoter 1) | | (intron 2) | NO: 11 | NO: 25 |
| Plasmid | SEQ ID NO: | NA | SEQ ID NO: | SEQ ID | SEQ ID |
| 11-3 | 31 | | 24 | | |
| | (promoter 2) | | (intron1) | NO: 11 | NO: 30 |
| Plasmid | SEQ ID NO: | NA | SEQ ID NO: | SEQ ID | SEQ ID |
| 11-4 | 31 | | 29 | | |
| | (promoter 2) | | (intron 2) | NO: 11 | NO: 25 |

6.2. Preparation of rAAVs

rAAV vectors were prepared with a method similar to that described in Example 2 with Plasmids 11-1, 11-2, 11-3 and 11-4 as the transgene plasmid, and the resulted rAAVs were referred to as SMN11-1, SMN11-2, SMN11-3 and SMN11-4, respectively.

6.3. In Vivo Expression of hSMN1 by the rAAVs

The rAAVs SMN11-1, SMN11-2, SMN11-3 and SMN11-4 as well as Zolgensma were IV injected (single dose) into neonatal FVB mice at PND1-2. The dosing regimen was shown in Table 2 (n=6).

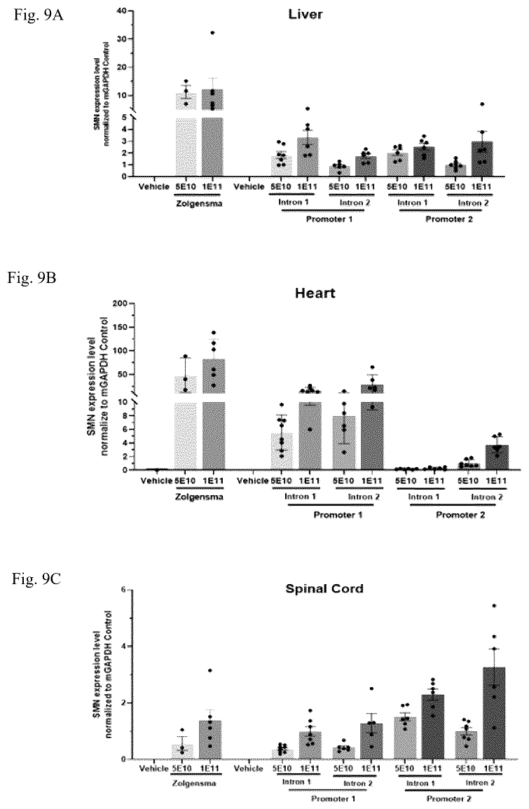

TABLE 2
Dosing regimen

| Groups | Test Article | Dose (vg/mouse) | Columns in FIG. 9 |
|---|---|---|---|
| 1 | Vehicle | 0 | Vehicle |
| 2 | Zolgensma | 5.00E+10 | Zolgensma-5E10 |
| 3 | | 1.00E+11 | Zolgensma-1E11 |
| 4 | SMN11-1 | 5.00E+10 | Promoter 1-intron 1-5E10 |
| 5 | | 1.00E+11 | Promoter 1-intron 1-1E11 |
| 6 | SMN11-2 | 5.00E+10 | Promoter 1-intron 2-5E10 |
| 7 | | 1.00E+11 | Promoter 1-intron 2-1E11 |
| 8 | SMN11-3 | 5.00E+10 | Promoter 2-intron 1-5E10 |
| 9 | | 1.00E+11 | Promoter 2-intron 1-1E11 |
| 10 | SMN11-4 | 5.00E+10 | Promoter 2-intron 2-5E10 |
| 11 | | 1.00E+11 | Promoter 2-intron 2-1E11 |
| 12 | Vehicle | 0 | Vehicle |

At Day 28 post injection, the tissues (heart, liver and spinal cord) were collected and analyzed for the expression levels of human SMN1. In particular, hSMN1 mRNA levels were detected. RT-qPCR method has been developed and validated with the samples from major peripheral and CNS tissues. Total RNA was isolated using the QIAGEN RNeasy Mini Kit with a minor modification using Qiazol and chloroform. Concentration of total RNA was determined by Nanodrop. For RT-qPCR analysis, Fast-RT-PCR was performed using lug total RNA together with QuantiTect Reverse Transcription Kit 400 (Cat.No. 205314) including genomic DNA elimination step. To perform qPCR, the cDNA was combined with qPCR reaction mixture (TaqMan Gene Expression Master Mix, 4369016), primers and probes (see Table 3) which corresponding to codon optimized hSMN1 transgene sequence (CoSMN11) and housekeeping gene GAPDH. In brief, 120 ng of total cDNA was used to perform qPCR in triplicate with TaqMan Gene Expression Master Mix (Thermos Fisher Scientific) and 900 nM of each primer and 450 nM of probe in 10 μl reaction in 384-well plates. The reactions were performed using the QuantStudio™ Real-Time PCR System. The relative mRNA expression of hSMN1 normalized to housekeeping gene GAPDH was reported.

TABLE 3
Primers and probes for the qPCR

| Gene | Primers and probes |
|---|---|
| Mouse | Forward primer: CCACTCACGGCAAATTCAAC |
| GAPDH | (SEQ ID NO: 41) |
| | Reverse primer: ACCAGTAGACTCCACGACATA |
| | (SEQ ID NO: 42) |
| | Probe: 5′-VIC-TTGTCATCAACGGGAAGCCCATC |
| | A-MGB-3′ (SEQ ID NO: 43) |
| SMN11 | Forward primer: CTATCATCCCTCCTCCTCCTC |
| | (SEQ ID NO: 44) |
| | Reverse primer: GCCTGAAGCCCATGTAGTAG |
| | (SEQ ID NO: 45) |
| | Probe: 5′-6-FAM-CTGGTACAT-ZEN-GAGCGGCT |
| | ACCACAC-IowaBlackFQ-3′ (SEQ ID NO: 46) |

As shown in FIG. 9, the results indeed suggested that the combinations has substantial impact on the expression of hSMN1 in different tissues. In particular, rAAVs comprising promoter 2 achieved a higher hSMN1 expression in spinal cord, among which the rAAV comprising promoter 2 and intron 2 achieved the highest, about 2-3 folds higher than Zolgensma; the rAAVs comprising promoter 1 achieved a higher expression than Zolgensma, but lower than rAAVs comprising promoter 2 (FIG. 9C). Regarding the liver and heart expression, the rAAVs of the present invention showed a lower (but meaningful) level than Zolgensma (FIGS. 9A and 9B).

Since the over-expression of hSMN1 in heart and liver could cause undesired effect, lower but functional expression could be safer but still effective in rescuing the SMA phenotype in heart and liver.

In addition, the expression levels of hSMN1 mRNA were dose dependent, higher expression level at the same dose than Zolgensma implicates that lower dose could be used to transduce the motor neurons in the spinal cord, reducing their degeneration and death, thereby, delaying the onset of the disease or even cure it.

Based on above results, the combination of CoSMN11 (SEQ ID NO: 11), promoter 2 (SEQ ID NO: 31) and intron 2 (SEQ ID NO: 29) was nominated as the lead transgene.

SEQUENCES
WT hSMN1
SEQ ID NO: 1
ATGGCGATGAGCAGCGGCGGCAGTGGTGGCGGCGTCCCGGAGCAGGAGGATTCCGTGCTGTTCCGGCGCGGCACA
GGCCAGAGCGATGATTCTGACATTTGGGATGATACAGCACTGATAAAAGCATATGATAAAGCTGTGGCTTCATTT
AAGCATGCTCTAAAGAATGGTGACATTTGTGAAACTTCGGGTAAACCAAAAACCACACCTAAAAGAAAACCTGCT
AAGAAGAATAAAAGCCAAAAGAAGAATACTGCAGCTTCCTTACAACAGTGGAAAGTTGGGGACAAATGTTCTGCC
ATTTGGTCAGAAGACGGTTGCATTTACCCAGCTACCATTGCTTCAATTGATTTTAAGAGAGAAACCTGTGTTGTG
GTTTACACTGGATATGGAAATAGAGAGGAGCAAAATCTGTCCGATCTACTTTCCCCAATCTGTGAAGTAGCTAAT
AATATAGAACAGAATGCTCAAGAGAATGAAAATGAAAGCCAAGTTTCAACAGATGAAAGTGAGAACTCCAGGTCT
CCTGGAAATAAATCAGATAACATCAAGCCCAAATCTGCTCCATGGAACTCTTTTCTCCCTCCACCACCCCCCATG
CCAGGGCCAAGACTGGGACCAGGAAAGCCAGGTCTAAAATTCAATGGCCCACCACCGCCACCGCCACCACCACCA
CCCCACTTACTATCATGCTGGCTGCCTCCATTTCCTTCTGGACCACCAATAATTCCCCCACCACCTCCCATATGT
CCAGATTCTCTTGATGATGCTGATGCTTTGGGAAGTATGTTAATTTCATGGTACATGAGTGGCTATCATACTGGC
TATTATATGGGTTTTAGACAAAATCAAAAAGAAGGAAGGTGCTCACATTCCTTAAATTAA
CoSMN2
SEQ ID NO: 2
ATGGCGATGAGCAGCGGCGGCAGTGGTGGCGGCGTCCCGGAGCAGGAGGATTCCGTGCTGTTCCGGCGCGGCACA
GGCCAGAGCGATGATTCTGACATTTGGGATGATACAGCACTGATAAAAGCATATGATAAAGCTGTGGCTTCATTT
AAGCATGCTCTAAAGAATGGTGACATTTGTGAAACTTCGGGTAAACCAAAAACCACACCTAAAAGAAAACCTGCT
AAGAAGAATAAAAGCCAAAAGAAGAATACTGCAGCTTCCTTACAACAGTGGAAAGTTGGGGACAAATGTTCTGCC
ATTTGGTCAGAAGACGGTTGCATTTACCCAGCTACCATTGCTTCAATTGATTTTAAGAGAGAAACCTGTGTTGTG
GTTTACACTGGATATGGAAATAGAGAGGAGCAAAATCTGTCCGATCTACTTTCCCCAATCTGTGAAGTAGCTAAT
AATATAGAACAAAATGCTCAAGAGAATGAAAATGAAAGCCAAGTTTCAACAGATGAAAGTGAGAACTCCAGGTCT
CCTGGAAATAAATCAGATAACATCAAGCCCAAATCTGCTCCATGGAACTCTTTTCTCCCTCCACCACCCCCCATG
CCAGGGCCAAGACTGGGACCAGGAAAGCCAGGTCTAAAATTCAATGGCCCACCACCGCCACCGCCACCACCACCA
CCCCACTTACTATCATGCTGGCTGCCTCCATTTCCTTCTGGACCACCAATAATTCCCCCACCACCTCCCATATGT
CCAGATTCTCTTGATGATGCTGATGCTTTGGGAAGTATGTTAATTTCATGGTACATGAGTGGCTATCATACTGGC
TATTATATGGGTTTCAGACAAAATCAAAAAGAAGGAAGGTGCTCACATTCCTTAAATTAA
CoSMN3
SEQ ID NO: 3
ATGGCAATGTCTTCCGGTGGATCTGGGGGCGGAGTCCCAGAACAGGAAGACTCCGTATTGTTCAGAAGAGGGACA
GGGCAATCTGACGACTCCGATATTTGGGATGACACGGCCCTCATTAAGGCATATGACAAGGCAGTGGCCAGTTTC
AAACATGCTCTGAAAAATGGCGACATTTGCGAAACATCCGGGAAGCCGAAGACAACACCAAAACGTAAGCCCGCA
AAGAAAAATAAGTCTCAGAAGAAGAACACTGCTGCCAGTCTTCAGCAGTGGAAAGTGGGAGACAAATGTTCCGCA
ATCTGGTCTGAGGACGGGTGTATTTACCCCGCCACGATCGCTTCTATCGACTTTAAGAGAGAAACTTGTGTGGTG
GTGTATACCGGCTACGGGAACCGCGAAGAGCAAAACCTGTCCGACCTGCTGTCCCCCATCTGCGAGGTGGCCAAC
AATATCGAGCAGAACGCTCAGGAGAACGAAAACGAGAGTCAAGTGTCTACAGATGAAAGCGAGAATTCTCGGTCT
CCAGGAAATAAATCTGACAATATCAAACCGAAAAGCGCCCCCTGGAATTCCTTTTTGCCCCCTCCACCTCCAATG
CCTGGGCCTAGACTGGGTCCTGGGAAGCCCGGCCTCAAGTTCAATGGACCGCCTCCCCCACCCCCCCCTCCCCCG
CCCCATCTCCTCAGTTGTTGGCTCCCCCCCTTCCCATCCGGGCCCCCAATCATTCCTCCGCCGCCCCCAATTTGT
CCAGACAGCCTGGACGACGCTGACGCTTTAGGGTCCATGCTCATCTCTTGGTATATGAGTGGCTATCATACCGGT
TATTACATGGGTTTCAGGCAGAACCAGAAGGAGGGCCGGTGTTCGCATAGCCTAAATTAG
CoSMN4
SEQ ID NO: 4
ATGGCCATGAGCAGCGGCGGATCTGGCGGCGGCGTGCCTGAGCAGGAGGATAGCGTGCTGTTCCGGAGAGGCACC
GGACAGTCCGATGATTCTGACATCTGGGACGACACCGCCCTGATCAAGGCCTACGACAAGGCCGTGGCTTCCTTC
AAGCACGCTCTGAAAAATGGCGACATCTGTGAAACCAGCGGCAAGCCCAAGACAACCCCTAAGAGAAAGCCTGCC
AAGAAAAACAAGTCTCAAAAGAAGAACACCGCCGCTTCTCTGCAGCAGTGGAAGGTGGGCGATAAGTGCAGCGCT
ATCTGGTCCGAGGACGGCTGTATCTACCCTGCCACCATCGCCAGCATCGACTTCAAGCGGGAAACATGCGTGGTG
GTCTACACAGGCTACGGCAACCGGGAAGAACAAAACCTGAGCGATCTGCTCAGCCCTATCTGCGAGGTGGCCAAC
AACATCGAGCAGAATGCCCAGGAAAACGAGAATGAAAGCCAGGTTTCCACCGACGAGTCTGAGAACAGCCGCAGC
CCCGGAAACAAGAGCGACAACATCAAGCCTAAGTCCGCCCCTTGGAACAGCTTTCTGCCTCCTCCGCCCCCCATG
CCTGGCCCTAGACTGGGCCCCGGCAAACCTGGACTGAAATTCAACGGCCCCCCCCCACCCCCCCCTCCTCCTCCA
CCTCACCTGCTGAGCTGCTGGCTGCCTCCCTTCCCTTCTGGGCCTCCAATTATCCCTCCACCTCCTCCTATCTGC
CCCGACAGCCTGGACGACGCCGACGCCCTGGGCAGCATGCTGATCAGCTGGTACATGAGCGGCTACCACACAGGC
TACTACATGGGCTTTAGACAGAACCAGAAAGAGGGCAGATGCAGCCATTCTCTGAATTGA
CoSMN5
SEQ ID NO: 5
ATGGCCATGTCCTCCGGTGGGTCAGGTGGGGGGGTCCCTGAACAGGAAGACAGTGTCTTGTTTCGACGCGGGACG
GGCCAAAGTGACGATTCTGATATTTGGGACGATACCGCACTCATTAAGGCTTACGATAAGGCTGTGGCGAGCTTC
AAGCACGCTTTGAAGAACGGAGATATTTGCGAGACGTCTGGCAAGCCGAAAACAACCCCGAAGCGCAAGCCCGCT
AAGAAGAATAAGTCCCAGAAAAAAAACACAGCTGCCAGTCTTCAGCAGTGGAAGGTGGGAGATAAATGCTCTGCC
ATATGGTCTGAGGATGGGTGTATTTACCCGGCAACAATCGCCAGCATCGACTTCAAACGAGAAACGTGCGTGGTC
GTTTATACCGGGTATGGGAACAGGGAGGAACAGAACCTCAGCGATCTTCTTTCTCCCATTTGTGAGGTGGCCAAC
AACATCGAACAAAATGCGCAGGAAAATGAGAATGAGAGTCAAGTGTCTACCGATGAAAGTGAAAATTCTCGCAGC
CCGGGAAACAAGTCTGACAATATCAAGCCCAAGTCTGCACCATGGAACAGTTTCTTGCCGCCTCCGCCACCGATG
CCGGGGCCTAGATTGGGGCCGGGCAAACCGGGACTTAAGTTCAACGGACCCCCTCCACCGCCCCCTCCTCCGCCA
CCGCACTTGTTGTCATGTTGGCTCCCTCCATTTCCCAGTGGTCCTCCTATCATACCACCTCCTCCGCCGATTTGT
CCCGATAGCCTTGATGACGCTGATGCTTTGGGATCCATGCTTATCTCTTGGTATATGTCAGGATACCATACCGGG
TATTACATGGGATTCAGACAGAACCAGAAAGAGGGGCGGTGCAGCCACTCCCTCAACTGA
COSMN6
SEQ ID NO: 6
ATGGCCATGTCTAGTGGCGGATCTGGTGGCGGAGTGCCCGAGCAAGAAGATAGCGTCCTGTTCAGAAGAGGCACC
GGCCAGAGCGACGACAGCGACATCTGGGATGATACAGCCCTGATCAAGGCCTACGACAAGGCCGTGGCCAGCTTT
AAGCACGCCCTGAAGAACGGCGATATCTGCGAGACAAGCGGCAAGCCCAAGACCACACCTAAGAGAAAGCCCGCC
AAGAAGAACAAGAGCCAGAAGAAGAATACCGCCGCCAGCCTGCAGCAGTGGAAAGTGGGCGATAAGTGCAGCGCC
ATTTGGAGCGAGGACGGCTGTATCTACCCTGCCACAATCGCCAGCATCGACTTCAAGCGGGAAACCTGCGTGGTG
GTGTACACAGGCTACGGCAACAGAGAGGAACAGAACCTGAGCGACCTGCTGAGCCCAATTTGCGAGGTGGCCAAC
AACATCGAGCAGAACGCCCAAGAGAACGAGAACGAGTCCCAGGTGTCCACCGACGAGAGCGAGAATAGCAGAAGC
CCCGGCAACAAGAGCGACAACATCAAGCCTAAGAGCGCCCCTTGGAACAGCTTCCTGCCTCCTCCTCCACCAATG
CCTGGACCTAGACTCGGACCTGGAAAGCCCGGCCTGAAGTTCAATGGACCTCCACCACCGCCACCACCTCCGCCT
CCACATCTTCTGTCTTGTTGGCTGCCTCCATTTCCTAGCGGCCCTCCAATCATCCCGCCACCTCCACCTATCTGC
CCCGACAGTCTGGATGATGCTGATGCCCTGGGCTCCATGCTGATCTCTTGGTACATGAGCGGCTACCACACCGGC
TACTACATGGGCTTCAGACAGAACCAGAAAGAGGGCCGTTGCAGCCACAGCCTGAACTGA
CoSMN7
SEQ ID NO: 7
ATGGCTATGAGCAGCGGAGGAAGCGGAGGCGGAGTGCCCGAACAAGAGGATTCCGTGCTGTTTAGAAGGGGAACC
GGACAGTCCGATGATAGCGACATCTGGGACGACACCGCTCTGATCAAGGCCTACGACAAAGCCGTGGCCTCCTTC
AAGCATGCTCTGAAGAACGGCGACATCTGCGAAACCAGCGGCAAGCCCAAAACAACCCCCAAGAGGAAGCCCGCC
AAGAAGAACAAGTCCCAGAAGAAGAACACCGCCGCTTCTCTGCAGCAGTGGAAGGTGGGCGACAAGTGTAGCGCC
ATCTGGTCCGAGGATGGCTGCATCTATCCCGCCACCATTGCCTCCATCGACTTCAAGAGGGAGACATGCGTGGTG
GTGTACACCGGCTACGGCAATAGAGAGGAACAGAATCTGTCCGATCTGCTGAGCCCTATCTGCGAGGTGGCCAAC
AACATTGAGCAGAACGCCCAAGAGAACGAGAACGAAAGCCAAGTGTCCACCGACGAGAGCGAAAACAGCAGAAGC
CCCGGCAACAAGAGCGACAACATCAAGCCCAAGTCCGCCCCTTGGAACTCCTTTCTGCCCCCTCCTCCCCCTATG
CCCGGCCCTAGACTGGGACCCGGCAAGCCCGGACTGAAGTTCAACGGACCCCCCCCTCCCCCCCCCCCCCCTCCT
CCTCATCTGCTGAGCTGTTGGCTCCCTCCTTTCCCCAGCGGCCCTCCTATCATCCCTCCTCCTCCTCCCATCTGC
CCCGATTCTCTGGATGATGCCGATGCTCTGGGCAGCATGCTGATCAGCTGGTACATGAGCGGCTATCACACCGGC
TACTACATGGGCTTCAGACAGAACCAGAAGGAGGGCAGATGCTCCCACTCTCTGAACTGA
CoSMN8
SEQ ID NO: 8
ATGGCTATGTCCTCTGGAGGGTCCGGCGGAGGCGTGCCGGAGCAGGAAGACTCCGTTCTGTTCCGCCGTGGCACA
GGGCAGTCAGACGATTCCGACATCTGGGATGACACTGCCCTCATCAAGGCCTACGATAAGGCTGTCGCTAGCTTT
AAGCATGCCCTGAAGAACGGAGACATCTGTGAGACATCCGGTAAGCCCAAGACCACGCCCAAGCGGAAGCCCGCC
AAAAAGAACAAGAGTCAGAAAAAGAACACCGCTGCATCTCTTCAACAGTGGAAAGTGGGCGACAAGTGCTCCGCT
ATCTGGAGCGAGGACGGGTGCATCTATCCAGCTACCATCGCATCTATCGACTTTAAGCGCGAGACCTGCGTAGTG
GTCTATACTGGTTACGGCAATAGGGAGGAACAGAACTTGTCTGATCTTCTGTCCCCGATCTGCGAAGTGGCCAAT
AACATTGAACAAAACGCCCAGGAGAACGAGAACGAGAGCCAGGTCTCCACGGACGAGAGCGAGAACAGTCGCTCC
CCTGGCAACAAGTCCGACAACATCAAACCGAAGTCTGCGCCCTGGAACTCATTCCTCCCACCTCCGCCCCCAATG
CCTGGTCCCCGCCTGGGCCCCGGCAAGCCGGGGCTCAAGTTTAATGGACCTCCCCCGCCCCCACCCCCACCCCCT
CCCCACCTGCTCTCTTGCTGGTTGCCCCCATTCCCCTCCGGGCCACCTATCATTCCCCCACCGCCTCCAATCTGC
CCAGACTCTCTGGACGATGCCGATGCCCTGGGCTCTATGCTGATCTCCTGGTATATGTCCGGCTATCATACCGGC
TATTACATGGGATTCCGCCAGAACCAAAAGGAGGGCCGCTGTAGCCACTCCCTGAACTGA
CoSMN9
SEQ ID NO: 9
ATGGCGATGAGCAGCGGCGGCAGTGGTGGCGGCGTCCCGGAGCAGGAGGATTCCGTGCTGTTCCGGCGCGGCACA
GGCCAGAGCGATGATTCTGACATTTGGGATGATACAGCACTGATAAAAGCATATGATAAAGCTGTGGCTTCATTT
AAGCATGCTCTAAAGAATGGTGACATTTGTGAAACTTCGGGTAAACCAAAAACCACACCTAAAAGAAAACCTGCT
AAGAAGAATAAAAGCCAAAAGAAGAATACTGCAGCTTCCTTACAACAGTGGAAAGTTGGGGACAAATGTTCTGCC
ATTTGGTCAGAAGACGGTTGCATTTACCCAGCTACCATTGCTTCAATTGATTTTAAGAGAGAAACCTGTGTTGTG
GTTTACACTGGATATGGAAATAGAGAGGAGCAAAATCTGTCCGATCTACTTTCCCCAATCTGTGAAGTAGCTAAT
AATATAGAACAGAATGCTCAAGAGAATGAAAATGAAAGCCAAGTTTCAACAGATGAAAGTGAGAACTCCAGGTCT
CCTGGAAATAAATCAGATAACATCAAGCCCAAATCTGCTCCATGGAACTCTTTTCTCCCTCCACCACCCCCCATG
CCAGGGCCAAGACTGGGACCAGGAAAGCCAGGTCTAAAATTCAATGGCCCACCACCGCCACCGCCACCACCACCA
CCCCACTTACTATCATGCTGGCTGCCTCCATTTCCTTCTGGACCACCAATAATTCCCCCACCACCTCCCATATGT
CCAGATTCTCTTGATGATGCTGATGCTTTGGGAAGTATGTTAATTTCATGGTACATGAGTGGCTATCATACTGGC
TATTATATGGGTTTTAGACAAAATCAAAAAGAAGGAAGGTGCTCACATTCCTTAAATTAA
CoSMN10
SEQ ID NO: 10
ATGGCCATGTCGTCCGGAGGCTCCGGTGGTGGGGTGCCTGAGCAAGAGGATTCCGTCCTCTTCAGGCGCGGGACC
GGGCAAAGTGATGACTCGGATATCTGGGACGATACTGCCTTAATCAAAGCCTACGATAAAGCCGTTGCTAGCTTT
AAACACGCTCTGAAGAATGGCGACATCTGCGAGACAAGTGGGAAGCCCAAGACCACCCCAAAGCGGAAGCCTGCA
AAAAAGAATAAGTCACAGAAAAAAAACACAGCCGCGAGCCTGCAGCAGTGGAAGGTCGGCGATAAGTGTTCTGCC
ATTTGGTCTGAGGACGGTTGTATCTACCCTGCGACCATCGCCTCAATTGACTTTAAACGGGAGACCTGCGTGGTA
GTCTACACAGGCTACGGCAACCGCGAGGAACAGAACCTGTCAGATTTGCTTAGCCCGATCTGTGAGGTGGCCAAT
AACATTGAACAGAATGCACAGGAAAATGAAAATGAATCCCAGGTGAGCACCGACGAGTCTGAAAATTCTCGGTCC
CCCGGTAATAAATCGGACAATATAAAACCAAAAAGCGCCCCGTGGAACTCTTTCCTCCCACCTCCTCCCCCCATG
CCAGGGCCACGCCTTGGCCCAGGTAAGCCGGGCCTCAAGTTCAACGGCCCCCCACCCCCCCCCCCTCCCCCTCCC
CCGCACTTACTGAGCTGTTGGCTACCCCCATTTCCATCAGGCCCACCCATCATTCCGCCACCCCCTCCCATCTGT
CCTGATTCACTAGATGACGCGGATGCCCTCGGCTCTATGCTAATCTCGTGGTATATGAGCGGCTATCACACAGGT
TACTACATGGGCTTCCGGCAAAATCAGAAAGAGGGCCGCTGCTCGCACTCTCTGAACTAA
CoSMN11
SEQ ID NO: 11
ATGGCCATGAGCAGCGGCGGCAGCGGCGGCGGCGTGCCTGAGCAGGAGGACAGCGTGCTGTTCAGGAGGGGCACC
GGCCAGAGCGACGACAGCGACATCTGGGACGACACCGCCCTGATCAAGGCCTACGACAAGGCCGTGGCCAGCTTC
AAGCACGCCCTGAAGAACGGCGACATCTGCGAGACCAGCGGCAAGCCTAAGACCACCCCTAAGAGGAAGCCTGCC
AAGAAGAACAAGAGCCAGAAGAAGAACACCGCCGCCAGCCTGCAGCAGTGGAAGGTGGGCGACAAGTGCAGCGCC
ATCTGGAGCGAGGACGGCTGCATCTACCCTGCCACCATCGCCAGCATCGACTTCAAGAGGGAGACCTGCGTGGTG
GTGTACACCGGCTACGGCAACAGGGAGGAGCAGAACCTGAGCGACCTGCTGAGCCCTATCTGCGAGGTGGCCAAC
AACATCGAGCAGAACGCCCAGGAGAACGAGAACGAGAGCCAGGTGAGCACCGACGAGAGCGAGAACAGCAGGAGC
CCTGGCAACAAGAGCGACAACATCAAGCCTAAGAGCGCCCCTTGGAACAGCTTCCTGCCTCCTCCTCCTCCTATG
CCTGGCCCTAGGCTGGGCCCTGGCAAGCCTGGCCTGAAGTTCAACGGCCCTCCTCCTCCTCCTCCTCCTCCTCCT
CCTCACCTGCTGAGCTGCTGGCTGCCTCCTTTCCCTAGCGGCCCTCCTATCATCCCTCCTCCTCCTCCTATCTGC
CCTGACAGCCTGGACGACGCCGACGCCCTGGGCAGCATGCTGATCAGCTGGTACATGAGCGGCTACCACACCGGC
TACTACATGGGCTTCAGGCAGAACCAGAAGGAGGGCAGGTGCAGCCACAGCCTGAACTGA
CoSMN12
SEQ ID NO: 12
ATGGCTATGTCCAGCGGTGGATCTGGGGGGGGCGTTCCCGAGCAGGAGGACTCCGTGCTGTTCCGCAGGGGTACT
GGCCAGAGTGATGACTCTGACATATGGGATGACACCGCCCTCATTAAGGCTTACGACAAAGCAGTGGCCAGTTTC
AAGCACGCTCTGAAAAACGGTGACATTTGTGAGACTTCTGGCAAACCTAAAACCACTCCTAAAAGAAAACCCGCT
AAGAAGAACAAATCACAAAAGAAGAATACAGCCGCCTCCTTACAGCAGTGGAAGGTGGGCGACAAGTGCTCAGCT
ATCTGGTCCGAGGACGGGTGCATTTACCCTGCCACCATCGCTTCAATCGATTTCAAAAGGGAGACCTGCGTGGTT
GTGTATACTGGGTACGGAAATCGAGAAGAACAGAATCTGTCCGATCTGCTCTCCCCCATATGCGAGGTCGCAAAC
AACATAGAACAGAATGCACAGGAGAACGAAAATGAGTCACAGGTTTCAACTGATGAAAGTGAGAATAGTAGGTCC
CCTGGTAATAAAAGCGACAACATAAAGCCCAAAAGCGCACCATGGAATAGCTTCCTGCCACCCCCCCCACCAATG
CCAGGGCCAAGGCTGGGGCCAGGCAAGCCGGGTCTAAAGTTTAATGGTCCGCCCCCACCGCCCCCCCCACCCCCA
CCCCACTTGCTGTCATGTTGGCTTCCGCCATTCCCTTCCGGTCCACCTATCATTCCTCCACCCCCACCAATTTGC
CCAGACAGCTTAGACGACGCAGACGCCTTGGGCTCGATGCTGATCAGCTGGTATATGTCGGGATACCATACAGGC
TACTATATGGGTTTCCGTCAGAACCAAAAGGAAGGCCGGTGTTCCCATTCCCTGAATTGA
CoSMN13
SEQ ID NO: 13
ATGGCCATGTCTTCAGGAGGATCAGGTGGGGGCGTCCCTGAACAGGAGGACTCTGTGCTCTTCCGGCGAGGTACG
GGCCAAAGCGATGACAGCGACATTTGGGATGACACCGCGCTCATCAAGGCCTATGATAAAGCTGTTGCATCCTTC
AAGCACGCTCTTAAAAATGGTGACATTTGTGAAACTTCCGGGAAACCCAAAACCACACCCAAGAGGAAGCCCGCC
AAGAAGAATAAATCTCAGAAGAAAAACACTGCTGCCTCCCTGCAGCAGTGGAAGGTGGGGGACAAGTGCAGCGCT
ATATGGTCTGAAGATGGCTGCATCTATCCAGCAACCATTGCGTCCATTGACTTCAAAAGAGAGACCTGTGTGGTG
GTATACACAGGATATGGCAACCGGGAGGAGCAAAACCTGAGCGACCTGCTATCACCCATTTGTGAGGTCGCCAAC
AACATCGAGCAGAATGCCCAGGAAAACGAGAATGAAAGCCAAGTTTCCACAGATGAAAGTGAGAACTCCAGGAGT
CCAGGGAACAAGTCAGACAATATAAAACCAAAAAGTGCACCTTGGAATAGCTTTCTCCCTCCACCACCACCCATG
CCTGGGCCCCGTTTGGGTCCTGGCAAGCCTGGCCTGAAGTTTAATGGACCTCCTCCCCCACCACCACCACCGCCC
CCTCATTTATTGAGCTGCTGGCTGCCCCCCTTTCCTAGTGGACCGCCCATCATCCCCCCGCCACCTCCAATCTGC
CCTGATTCTCTGGATGATGCTGATGCATTAGGCAGCATGCTGATATCCTGGTACATGAGTGGCTACCACACTGGT
TACTATATGGGGTTCCGCCAGAACCAGAAAGAAGGCAGATGTTCGCATTCACTTAATTGA
CoSMN14
SEQ ID NO: 14
ATGGCCATGAGCTCAGGGGGTTCTGGAGGCGGTGTACCAGAACAAGAAGACTCTGTTCTCTTCAGGAGAGGAACA
GGGCAATCTGATGACAGCGACATCTGGGACGACACTGCTTTAATAAAGGCGTATGACAAGGCTGTCGCTTCCTTC
AAGCATGCACTTAAAAATGGAGACATTTGTGAGACTAGTGGGAAACCTAAGACAACTCCAAAGAGGAAGCCTGCC
AAGAAGAACAAAAGCCAGAAAAAAAACACAGCAGCATCCTTGCAGCAGTGGAAAGTGGGTGATAAATGTTCAGCA
ATATGGAGCGAAGATGGCTGCATCTACCCAGCCACCATTGCCAGCATTGATTTTAAAAGAGAGACCTGTGTGGTC
GTTTACACCGGCTATGGCAACCGGGAGGAGCAAAACCTGAGTGACCTGTTGTCTCCGATCTGTGAAGTGGCCAAC
AACATTGAGCAGAATGCTCAGGAGAATGAAAATGAGTCCCAGGTGTCCACCGATGAATCTGAGAACTCCCGTTCA
CCCGGAAATAAATCGGACAACATCAAGCCCAAGAGTGCGCCCTGGAATTCCTTCCTTCCTCCGCCGCCTCCCATG
CCTGGGCCTCGATTAGGCCCTGGGAAGCCAGGGCTCAAGTTCAATGGCCCACCCCCACCACCACCTCCACCGCCC
CCCCACCTACTGTCGTGCTGGCTGCCTCCTTTTCCTTCTGGTCCCCCCATTATCCCTCCTCCCCCACCCATCTGC
CCAGACAGTCTGGATGATGCTGATGCCCTGGGAAGCATGCTGATCTCCTGGTACATGAGTGGATATCACACGGGT
TACTACATGGGCTTTCGGCAGAACCAGAAGGAGGGCCGCTGCAGTCATAGCCTCAACTGA
CoSMN15
SEQ ID NO: 15
ATGGCCATGAGCTCCGGGGGAAGTGGAGGGGGAGTTCCTGAGCAAGAAGACAGCGTCCTCTTTAGGAGGGGTACA
GGCCAATCCGATGATTCAGATATTTGGGACGACACTGCTCTGATTAAAGCATACGACAAAGCGGTGGCAAGTTTC
AAGCATGCATTGAAAAATGGTGATATATGCGAGACTTCAGGCAAGCCTAAAACGACACCCAAGAGAAAGCCAGCT
AAGAAAAACAAGAGCCAAAAGAAGAACACAGCCGCCAGCCTCCAGCAGTGGAAGGTCGGTGATAAATGTTCAGCT
ATATGGTCAGAGGACGGGTGTATTTATCCAGCAACCATCGCCAGCATCGACTTCAAACGGGAGACCTGTGTAGTG
GTTTACACCGGATACGGCAACCGGGAAGAGCAGAACCTGTCCGACCTTCTGTCTCCAATCTGTGAGGTGGCTAAC
AATATCGAGCAGAATGCCCAGGAAAATGAGAATGAATCCCAGGTGTCGACGGATGAGTCGGAAAACTCTCGAAGT
CCCGGGAATAAGTCCGATAATATCAAACCAAAAAGTGCCCCCTGGAACTCTTTCCTCCCACCTCCTCCACCTATG
CCCGGGCCCCGCCTGGGCCCCGGGAAGCCTGGATTGAAGTTTAACGGCCCCCCTCCGCCACCCCCGCCCCCACCG
CCTCATCTGCTAAGTTGCTGGCTGCCTCCGTTTCCGTCTGGCCCACCGATTATCCCCCCTCCGCCACCCATTTGC
CCAGACAGCCTTGACGATGCGGACGCCTTAGGTTCCATGTTAATCAGCTGGTATATGTCCGGCTATCACACCGGC
TACTACATGGGATTCAGACAGAATCAGAAGGAAGGCCGCTGCAGCCACTCTCTGAACTGA
CoSMN16
SEQ ID NO: 16
ATGGCAATGAGTAGTGGTGGTAGTGGTGGTGGAGTGCCTGAGCAGGAAGATTCCGTGCTGTTCCGGAGAGGCACA
GGCCAGTCTGACGACTCTGATATCTGGGATGACACCGCTCTGATCAAGGCCTACGACAAGGCCGTGGCCAGCTTT
AAGCACGCCCTGAAGAATGGCGACATTTGTGAAACCTCTGGCAAACCCAAGACCACCCCTAAGCGGAAGCCTGCC
AAGAAGAACAAGTCCCAGAAGAAAAACACCGCCGCTTCTCTGCAGCAATGGAAGGTGGGCGACAAGTGCTCTGCC
ATCTGGTCCGAGGACGGCTGCATCTACCCTGCTACCATCGCCTCTATCGACTTCAAGAGAGAGACATGTGTGGTC
GTGTACACCGGCTACGGCAACCGGGAGGAACAGAACCTGTCCGACCTGCTCTCTCCCATCTGCGAGGTGGCTAAC
AACATCGAGCAAAACGCCCAGGAAAACGAGAACGAGTCCCAGGTGTCCACCGACGAATCCGAGAATTCCCGCTCT
CCTGGCAATAAGTCCGACAACATCAAGCCTAAAAGCGCTCCTTGGAACTCCTTCCTGCCTCCACCTCCTCCAATG
CCTGGTCCTAGACTGGGCCCCGGCAAGCCCGGCCTGAAGTTCAACGGCCCTCCTCCACCTCCCCCGCCCCCACCC
CCTCACCTGCTGTCCTGCTGGCTGCCTCCTTTCCCTTCTGGCCCCCCCATCATCCCTCCACCTCCTCCTATCTGC
CCTGATAGCCTGGACGATGCCGACGCTCTGGGATCTATGCTGATCTCCTGGTATATGTCCGGATACCACACCGGC
TACTACATGGGCTTCAGACAGAACCAGAAAGAGGGCAGATGCAGCCATTCCCTGAACTGA
CoSMN17
SEQ ID NO: 17
ATGGCAATGAGTTCCGGGGGGTCAGGTGGAGGTGTGCCAGAACAAGAAGATAGTGTGCTCTTCCGTCGGGGCACA
GGCCAATCAGATGATAGTGACATCTGGGATGACACCGCACTTATCAAAGCATATGATAAAGCAGTAGCTTCTTTC
AAGCATGCTCTAAAAAATGGAGACATATGTGAAACGTCAGGGAAACCAAAAACAACTCCTAAAAGGAAGCCTGCG
AAGAAAAACAAGTCTCAGAAGAAGAACACTGCAGCCAGTTTGCAGCAGTGGAAAGTGGGAGACAAGTGCAGTGCT
ATTTGGTCTGAGGATGGCTGCATTTACCCTGCTACCATAGCATCCATTGATTTTAAGAGAGAGACTTGTGTCGTT
GTCTACACAGGTTATGGCAACAGAGAAGAGCAGAACCTCTCAGACTTACTTAGCCCCATTTGTGAGGTTGCCAAC
AATATTGAACAGAATGCTCAAGAGAATGAAAATGAGAGTCAGGTATCTACAGATGAATCTGAGAACAGCAGGAGC
CCTGGAAATAAATCAGACAATATAAAGCCCAAAAGCGCCCCCTGGAATTCTTTTTTGCCTCCTCCGCCCCCAATG
CCAGGTCCACGACTGGGCCCAGGAAAACCTGGGCTAAAGTTTAATGGGCCTCCTCCACCCCCCCCTCCGCCACCG
CCTCATCTGCTGTCCTGCTGGCTTCCACCATTTCCAAGTGGACCTCCCATCATTCCTCCCCCACCACCAATCTGT
CCAGATTCGTTAGACGATGCAGATGCCCTGGGATCTATGCTGATATCCTGGTACATGTCTGGATACCACACAGGG
TATTATATGGGTTTCAGACAAAACCAGAAAGAAGGACGCTGCTCACACAGCTTGAACTGA
CoSMN18
SEQ ID NO: 18
ATGGCCATGAGCTCCGGTGGTAGTGGCGGAGGCGTACCTGAACAAGAAGACTCTGTTTTGTTTCGGAGGGGCACC
GGCCAAAGCGATGATTCAGATATATGGGACGACACCGCGTTAATCAAGGCCTACGATAAAGCTGTGGCATCTTTC
AAACATGCATTGAAGAATGGAGACATCTGCGAGACCAGCGGAAAACCCAAGACAACGCCAAAGCGCAAACCTGCC
AAGAAGAATAAGAGCCAGAAGAAAAATACTGCTGCCTCACTGCAGCAGTGGAAGGTGGGGGATAAATGTAGCGCC
ATATGGAGCGAGGATGGCTGCATCTATCCAGCTACAATTGCATCCATTGACTTCAAGAGGGAAACTTGCGTGGTG
GTCTACACCGGATATGGTAACAGAGAGGAACAGAATCTCTCTGACCTTCTGTCCCCAATTTGCGAGGTCGCCAAC
AACATAGAGCAGAATGCCCAGGAGAACGAAAATGAATCACAAGTTTCGACAGATGAAAGTGAGAACAGCCGATCC
CCGGGGAACAAATCTGACAACATCAAACCAAAGAGTGCGCCCTGGAATTCATTTCTGCCCCCACCCCCTCCCATG
CCTGGACCACGTCTAGGTCCTGGGAAACCTGGCCTTAAGTTCAATGGCCCCCCTCCTCCACCCCCACCTCCACCG
CCTCACCTGCTGTCTTGTTGGCTGCCGCCCTTCCCCAGTGGGCCGCCCATCATTCCTCCGCCCCCCCCAATCTGT
CCAGACAGCCTGGATGACGCTGATGCACTAGGCTCCATGCTCATTTCCTGGTATATGTCTGGGTATCACACTGGA
TACTACATGGGGTTTCGGCAGAACCAGAAAGAGGGCAGATGTTCCCATTCTCTCAACTGA
CoSMN19
SEQ ID NO: 19
ATGGCCATGTCTTCGGGCGGCTCGGGTGGCGGTGTCCCCGAACAGGAGGACTCCGTGCTGTTCCGGCGCGGGACT
GGACAGAGCGATGACAGTGACATATGGGATGACACCGCCCTGATCAAGGCGTACGACAAAGCCGTAGCTTCTTTC
AAGCACGCCCTGAAGAATGGGGACATCTGCGAGACAAGCGGCAAGCCCAAAACCACTCCTAAGCGCAAGCCTGCC
AAGAAAAACAAGTCCCAGAAGAAGAACACGGCAGCCAGCTTGCAACAGTGGAAGGTGGGTGACAAGTGTTCTGCT
ATCTGGTCTGAAGACGGCTGTATTTATCCAGCGACCATCGCATCCATTGACTTTAAGAGAGAGACCTGCGTGGTG
GTGTACACCGGCTACGGCAACCGCGAGGAGCAGAACCTGTCCGACCTGCTGTCGCCCATCTGCGAGGTGGCCAAC
AACATTGAGCAGAATGCGCAGGAGAATGAGAACGAGTCCCAGGTGTCCACCGACGAGAGCGAGAACTCGCGTAGC
CCAGGCAACAAGTCCGACAACATCAAACCCAAGTCAGCTCCGTGGAACTCCTTCCTGCCTCCTCCACCGCCAATG
CCTGGACCCCGCCTGGGTCCGGGCAAGCCCGGTCTCAAATTTAACGGCCCTCCACCCCCCCCCCCGCCTCCCCCT
CCCCACCTGCTTTCATGCTGGCTTCCGCCCTTCCCGTCTGGGCCGCCCATCATCCCGCCTCCGCCCCCCATCTGC
CCAGATTCCTTGGATGACGCGGATGCTCTGGGATCTATGTTAATCAGTTGGTACATGAGCGGCTACCACACGGGC
TATTACATGGGCTTCCGCCAGAACCAGAAGGAGGGCAGGTGTAGCCACTCCCTCAACTGA
CoSMN20
SEQ ID NO: 20
ATGGCCATGTCGTCAGGGGGCTCAGGAGGTGGAGTTCCTGAGCAGGAGGATAGTGTCCTGTTCCGAAGGGGCACC
GGACAAAGTGATGACTCTGACATTTGGGATGACACCGCGTTGATAAAGGCATATGACAAAGCAGTGGCTAGCTTC
AAACATGCTCTTAAAAATGGTGACATCTGTGAAACTTCTGGGAAGCCAAAAACGACACCCAAAAGGAAGCCGGCC
AAGAAGAATAAATCTCAAAAGAAAAACACTGCAGCGTCCCTGCAGCAGTGGAAGGTTGGAGACAAGTGTTCAGCC
ATCTGGAGCGAAGATGGCTGCATCTATCCTGCCACCATCGCCTCCATTGACTTTAAACGGGAGACCTGTGTAGTG
GTCTACACTGGCTATGGTAATAGAGAGGAGCAAAATCTTTCAGACCTCCTCTCTCCCATTTGTGAGGTGGCCAAC
AACATCGAGCAGAATGCCCAGGAAAATGAGAATGAAAGCCAGGTGAGTACAGATGAAAGTGAAAACTCCCGCTCT
CCCGGCAACAAGAGTGACAACATTAAGCCCAAGTCAGCTCCCTGGAACTCCTTTTTACCACCACCTCCGCCCATG
CCTGGGCCTCGGCTAGGCCCAGGTAAACCCGGTCTGAAGTTTAATGGGCCTCCACCTCCACCCCCGCCTCCCCCA
CCCCACCTGCTCAGCTGCTGGCTGCCCCCTTTCCCTTCGGGACCGCCCATAATTCCACCTCCACCACCGATCTGC
CCCGACAGCTTAGATGATGCTGATGCACTGGGGAGCATGCTGATCTCCTGGTACATGAGCGGCTACCACACAGGA
TACTACATGGGCTTCAGACAGAACCAGAAAGAGGGCCGCTGCTCCCATTCTTTGAACTGA
hSMN1 amino acid sequence
SEQ ID NO: 21
MAMSSGGSGGGVPEQEDSVLFRRGTGQSDDSDIWDDTALIKAYDKAVASFKHALKNGDICETSGKPKTTPKRKPA
KKNKSQKKNTAASLQQWKVGDKCSAIWSEDGCIYPATIASIDFKRETCVVVYTGYGNREEQNLSDLLSPICEVAN
NIEQNAQENENESQVSTDESENSRSPGNKSDNIKPKSAPWNSFLPPPPPMPGPRLGPGKPGLKFNGPPPPPPPPP
PHLLSCWLPPFPSGPPIIPPPPPICPDSLDDADALGSMLISWYMSGYHTGYYMGFRQNQKEGRCSHSLN*
CMV IE enhancer
SEQ ID NO: 22
CTAGTCGACATTGATTATTGACTAGTTATTAATAGTAATCAATTACGGGGTCATTAGTTCATAGCCCATATATGG
AGTTCCGCGTTACATAACTTACGGTAAATGGCCCGCCTGGCTGACCGCCCAACGACCCCCGCCCATTGACGTCAA
TAATGACGTATGTTCCCATAGTAACGCCAATAGGGACTTTCCATTGACGTCAATGGGTGGAGTATTTACGGTAAA
CTGCCCACTTGGCAGTACATCAAGTGTATCATATGCCAAGTACGCCCCCTATTGACGTCAATGACGGTAAATGGC
CCGCCTGGCATTATGCCCAGTACATGACCTTATGGGACTTTCCTACTTGGCAGTACATCTACGTATTAGTCATCG
CTATTAC
chicken beta-actin core (CB) promoter
SEQ ID NO: 23
TGGTCGAGGTGAGCCCCACGTTCTGCTTCACTCTCCCCATCTCCCCCCCCTCCCCACCCCCAATTTTGTATTTAT
TTATTTTTTAATTATTTTGTGCAGCGATGGGGGGGGGGGGGGGGGGGGGGGGCGCGCCAGGCGGGGCGGGGGGGG
GCGAGGGGCGGGGCGGGGCGAGGCGGAGAGGTGCGGCGGCAGCCAATCAGAGCGGCGCGCTCCGAAAGTTTCCTT
TTATGGCGAGGCGGCGGCGGCGGCGGCCCTATAAAAAGCGAAGCGCGCGGCGGGCGG
PCI intron
SEQ ID NO: 24
GTAAGTATCAAGGTTACAAGACAGGTTTAAGGAGACCAATAGAAACTGGGCTTGTCGAGACAGAGAAGACTCTTG
CGTTTCTGATAGGCACCTATTGGTCTTACTGACATCCACTTTGCCTTTCTCTCCACAG
RGb polyA signal
SEQ ID NO: 25
GATCTTTTTCCCTCTGCCAAAAATTATGGGGACATCATGAAGCCCCTTGAGCATCTGACTTCTGGCTAATAAAGG
AAATTTATTTTCATTGCAATAGTGTGTTGGAATTTTTTGTGTCTCTCACTCG
5′ ITR105
SEQ ID NO: 26
CTGCGCGCTCGCTCGCTCACTGAGGCCGCCCGGGCAAAGCCCGGGCGTCGGGCGACCTTTGGTCGCCCGGCCTCA
GTGAGCGAGCGAGCGCGCAGAGAGGGAGTG
3′ ITR130
SEQ ID NO: 27
AGGAACCCCTAGTGATGGAGTTGGCCACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCGCGCAG
hPGK promoter
SEQ ID NO: 28
GGGGTTGGGGTTGCGCCTTTTCCAAGGCAGCCCTGGGTTTGCGCAGGGACGCGGCTGCTCTGGGCGTGGTTCCGG
GAAACGCAGCGGCGCCGACCCTGGGTCTCGCACATTCTTCACGTCCGTTCGCAGCGTCACCCGGATCTTCGCCGC
TACCCTTGTGGGCCCCCCGGCGACGCTTCCTGCTCCGCCCCTAAGTCGGGAAGGTTCCTTGCGGTTCGCGGCGTG
CCGGACGTGACAAACGGAAGCCGCACGTCTCACTAGTACCCTCGCAGACGGACAGCGCCAGGGAGCAATGGCAGC
GCGCCGACCGCGATGGGCTGTGGCCAATAGCGGCTGCTCAGCAGGGCGCGCCGAGAGCAGCGGCCGGGAAGGGGC
GGTGCGGGAGGCGGGGTGTGGGGCGGTAGTGTGGGCCCTGTTCCTGCCCGCGCGGTGTTCCGCATTCTGCAAGCC
TCCGGAGCGCACGTCGGCAGTCGGCTCCCTCGTTGACCGAATCACCGACCTCTCTCCCCAG
chimeric intron CBA + rabbit globin
SEQ ID NO: 29
GGAGTCGCTGCGCGCTGCCTTCGCCCCGTGCCCCGCTCCGCCGCCGCCTCGCGCCGCCCGCCCCGGCTCTGACTG
ACCGCGTTACTCCCACAGGTGAGCGGGCGGGACGGCCCTTCTCCTCCGGGCTGTAATTAGCGCTTGGTTTAATGA
CGGCTTGTTTCTTTTCTGTGGCTGCGTGAAAGCCTTGAGGGGCTCCGGGAGCTAGAGCCTCTGCTAACCATGTTC
ATGCCTTCTTCTTTTTCCTACAGCTCCTGGGCAACGTGCTGGTTATTGTGCTGTCTCATCATTTTGGCAAAGAAT
TT
bGH polyA signal
SEQ ID NO: 30
CTAGAGCTCGCTGATCAGCCTCGACTGTGCCTTCTAGTTGCCAGCCATCTGTTGTTTGCCCCTCCCCCGTGCCTT
CCTTGACCCTGGAAGGTGCCACTCCCACTGTCCTTTCCTAATAAAATGAGGAAATTGCATCGCATTGTCTGAGTA
GGTGTCATTCTATTCTGGGGGGTGGGGTGGGGCAGGACAGCAAGGGGGAGGATTGGGAAGACAATAGCAGGCATG
CTGGGGA
hSyn promoter
SEQ ID NO: 31
CTGCAGAGGGCCCTGCGTATGAGTGCAAGTGGGTTTTAGGACCAGGATGAGGCGGGGTGGGGGTGCCTACCTGAC
GACCGACCCCGACCCACTGGACAAGCACCCAACCCCCATTCCCCAAATTGCGCATCCCCTATCAGAGAGGGGGAG
GGGAAACAGGATGCGGCGAGGCGCGTGCGCACTGCCAGCTTCAGCACCGCGGACAGTGCCTTCGCCCCCGCCTGG
CGGCGCGCGCCACCGCCGCCTCAGCACTGAAGGCGCGCTGACGTCACTCGCCGGTCCCCCGCAAACTCCCCTTCC
CGGCCACCTTGGTCGCGTCCGCGCCGCCGCCGGCCCAGCCGGACCGCACCACGCGAGGCGCGAGATAGGGGGGCA
CGGGCGCGACCATCTGCGCTGCGGCGCCGGCGACTCAGCGCTGCCTCAGTCTGCGGTGGGCAGCGGAGGAGTCGT
GTCGTGCCTGAGAGCGCAG
AAV9 capsid amino acid sequence
SEQ ID NO: 32
MAADGYLPDWLEDNLSEGIREWWALKPGAPQPKANQQHODNARGLVLPGYKYLGPGNGLDKGEPVNAADAAALEH
DKAYDQQLKAGDNPYLKYNHADAEFQERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGYFDENRFHCHFSPRDWOR
LINNNWGFRPKRLNFKLFNIQVKEVTDNNGVKTIANNLTSTVQVFTDSDYQLPYVLGSAHEGCLPPFPADVFMIP
QYGYLTLNDGSQAVGRSSFYCLEYFPSQMLRTGNNFOFSYEFENVPFHSSYAHSQSLDRLMNPLIDQYLYYLSKT
INGSGQNQQTLKFSVAGPSNMAVQGRNYIPGPSYRQQRVSTTVTONNNSEFAWPGASSWALNGRNSLMNPGPAMA
SHKEGEDRFFPLSGSLIFGKOGTGRDNVDADKVMITNEEEIKTTNPVATESYGQVATNHQSAQAQAQTGWVONOG
ILPGMVWQDRDVYLQGPIWAKIPHTDGNFHPSPLMGGFGMKHPPPQILIKNTPVPADPPTAFNKDKLNSFITQYS
TGQVSVEIEWELQKENSKRWNPEIQYTSNYYKSNNVEFAVNTEGVYSEPRPIGTRYLTRNL
AAV2 Rep amino acid sequence
SEQ ID NO: 33
MPGFYEIVIKVPSDLDEHLPGISDSFVNWVAEKEWELPPDSDMDLNLIEQAPLTVAEKLORDELTEWRRVSKAPE
ALFFVQFEKGESYFHMHVLVETTGVKSMVLGRFLSQIREKLIQRIYRGIEPTLPNWFAVTKTRNGAGGGNKVVDE
CYIPNYLLPKTOPELQWAWINMEQYLSACLNLTERKRLVAQHLTHVSQTQEQNKENQNPNSDAPVIRSKTSARYM
ELVGWLVDKGITSEKQWIQEDQASYISFNAASNSRSQIKAALDNAGKIMSLTKTAPDYLVGOOPVEDISSNRIYK
ILELNGYDPQYAASVELGWATKKFGKRNTIWLFGPATTGKINIAEAIAHTVPFYGCVNWTNENFPENDCVDKMVI
WWEEGKMTAKVVESAKAILGGSKVRVDQKCKSSAQIDPTPVIVTSNTNMCAVIDGNSTTFEHQQPLQDRMFKFEL
TRRLDHDFGKVTKQEVKDFFRWAKDHVVEVEHEFYVKKGGAKKRPAPSDADISEPKRVRESVAQPSTSDAEASIN
YADRYQNKCSRHVGMNLMLFPCROCERMNONSNICFTHGQKDCLECFPVSESQPVSVVKKAYQKLCYIHHIMGKV
PDACTACDLVNVDLDDCIFEQ
AAV9 capsid coding sequence
SEQ ID NO: 34
ATGGCTGCCGATGGTTATCTTCCAGATTGGCTCGAGGACAACCTTAGTGAAGGAATTCGCGAGTGGTGGGCTTTG
AAACCTGGAGCCCCTCAACCCAAGGCAAATCAACAACATCAAGACAACGCTCGAGGTCTTGTGCTTCCGGGTTAC
AAATACCTTGGACCCGGCAACGGACTCGACAAGGGGGAGCCGGTCAACGCAGCAGACGCGGCGGCCCTCGAGCAC
GACAAGGCCTACGACCAGCAGCTCAAGGCCGGAGACAACCCGTACCTCAAGTACAACCACGCCGACGCCGAGTTC
CAGGAGCGGCTCAAAGAAGATACGTCTTTTGGGGGCAACCTCGGGCGAGCAGTCTTCCAGGCCAAAAAGAGGCTT
CTTGAACCTCTTGGTCTGGTTGAGGAAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGCTAAAAAGAGACTCAATTTCGGTCAG
ACTGGCGACACAGAGTCAGTCCCAGACCCTCAACCAATCGGAGAACCTCCCGCAGCCCCCTCAGGTGTGGGATCT
CTTACAATGGCTTCAGGTGGTGGCGCACCAGTGGCAGACAATAACGAAGGTGCCGATGGAGTGGGTAGTTCCTCG
GGAAATTGGCATTGCGATTCCCAATGGCTGGGGGACAGAGTCATCACCACCAGCACCCGAACCTGGGCCCTGCCC
ACCTACAACAATCACCTCTACAAGCAAATCTCCAACAGCACATCTGGAGGATCTTCAAATGACAACGCCTACTTC
GGCTACAGCACCCCCTGGGGGTATTTTGACTTCAACAGATTCCACTGCCACTTCTCACCACGTGACTGGCAGCGA
CTCATCAACAACAACTGGGGATTCCGGCCTAAGCGACTCAACTTCAAGCTCTTCAACATTCAGGTCAAAGAGGTT
ACGGACAACAATGGAGTCAAGACCATCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTTCCCAGCGGACGTTTTCATGATTCCT
CAGTACGGGTATCTGACGCTTAATGATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTTTGAGAACGTACCTTTCCATAGCAGC
TACGCTCACAGCCAAAGCCTGGACCGACTAATGAATCCACTCATCGACCAATACTTGTACTATCTCTCAAAGACT
ATTAACGGTTCTGGACAGAATCAACAAACGCTAAAATTCAGTGTGGCCGGACCCAGCAACATGGCTGTCCAGGGA
AGAAACTACATACCTGGACCCAGCTACCGACAACAACGTGTCTCAACCACTGTGACTCAAAACAACAACAGCGAA
TTTGCTTGGCCTGGAGCTTCTTCTTGGGCTCTCAATGGACGTAATAGCTTGATGAATCCTGGACCTGCTATGGCC
AGCCACAAAGAAGGAGAGGACCGTTTCTTTCCTTTGTCTGGATCTTTAATTTTTGGCAAACAAGGAACTGGAAGA
GACAACGTGGATGCGGACAAAGTCATGATAACCAACGAAGAAGAAATTAAAACTACTAACCCGGTAGCAACGGAG
TCCTATGGACAAGTGGCCACAAACCACCAGAGTGCCCAAGCACAGGCGCAGACCGGCTGGGTTCAAAACCAAGGA
ATACTTCCGGGTATGGTTTGGCAGGACAGAGATGTGTACCTGCAAGGACCCATTTGGGCCAAAATTCCTCACACG
GACGGCAACTTTCACCCTTCTCCGCTGATGGGAGGGTTTGGAATGAAGCACCCGCCTCCTCAGATCCTCATCAAA
AACACACCTGTACCTGCGGATCCTCCAACGGCCTTCAACAAGGACAAGCTGAACTCTTTCATCACCCAGTATTCT
ACTGGCCAAGTCAGCGTGGAGATCGAGTGGGAGCTGCAGAAGGAAAACAGCAAGCGCTGGAACCCGGAGATCCAG
TACACTTCCAACTATTACAAGTCTAATAATGTTGAATTTGCTGTTAATACTGAAGGTGTATATAGTGAACCCCGC
CCCATTGGCACCAGATACCTGACTCGTAATCTGTAA
AAV2 Rep coding sequence
SEQ ID NO: 35
ATGCCGGGGTTTTACGAGATTGTGATTAAGGTCCCCAGCGACCTTGACGAGCATCTGCCCGGCATTTCTGACAGC
TTTGTGAACTGGGTGGCCGAGAAGGAATGGGAGTTGCCGCCAGATTCTGACATGGATCTGAATCTGATTGAGCAG
GCACCCCTGACCGTGGCCGAGAAGCTGCAGCGCGACTTTCTGACGGAATGGCGCCGTGTGAGTAAGGCCCCGGAG
GCTCTTTTCTTTGTGCAATTTGAGAAGGGAGAGAGCTACTTCCACATGCACGTGCTCGTGGAAACCACCGGGGTG
AAATCCATGGTTTTGGGACGTTTCCTGAGTCAGATTCGCGAAAAACTGATTCAGAGAATTTACCGCGGGATCGAG
CCGACTTTGCCAAACTGGTTCGCGGTCACAAAGACCAGAAATGGCGCCGGAGGGGGGAACAAGGTGGTGGATGAG
TGCTACATCCCCAATTACTTGCTCCCCAAAACCCAGCCTGAGCTCCAGTGGGCGTGGACTAATATGGAACAGTAT
TTAAGCGCCTGTTTGAATCTCACGGAGCGTAAACGGTTGGTGGCGCAGCATCTGACGCACGTGTCGCAGACGCAG
GAGCAGAACAAAGAGAATCAGAATCCCAATTCTGATGCGCCGGTGATCAGATCAAAAACTTCAGCCAGGTACATG
GAGCTGGTCGGGTGGCTCGTGGACAAGGGGATTACCTCGGAGAAGCAGTGGATCCAGGAGGACCAGGCCTCATAC
ATCTCCTTCAATGCGGCCTCCAACTCGCGGTCCCAAATCAAGGCTGCCTTGGACAATGCGGGAAAGATTATGAGC
CTGACTAAAACCGCCCCCGACTACCTGGTGGGCCAGCAGCCCGTGGAGGACATTTCCAGCAATCGGATTTATAAA
ATTTTGGAACTAAACGGGTACGATCCCCAATATGCGGCTTCCGTCTTTCTGGGATGGGCCACGAAAAAGTTCGGC
AAGAGGAACACCATCTGGCTGTTTGGGCCTGCAACTACCGGGAAGACCAACATCGCGGAGGCCATAGCCCACACT
GTGCCCTTCTACGGGTGCGTAAACTGGACCAATGAGAACTTTCCCTTCAACGACTGTGTCGACAAGATGGTGATC
TGGTGGGAGGAGGGGAAGATGACCGCCAAGGTCGTGGAGTCGGCCAAAGCCATTCTCGGAGGAAGCAAGGTGCGC
GTGGACCAGAAATGCAAGTCCTCGGCCCAGATAGACCCGACTCCCGTGATCGTCACCTCCAACACCAACATGTGC
GCCGTGATTGACGGGAACTCAACGACCTTCGAACACCAGCAGCCGTTGCAAGACCGGATGTTCAAATTTGAACTC
ACCCGCCGTCTGGATCATGACTTTGGGAAGGTCACCAAGCAGGAAGTCAAAGACTTTTTCCGGTGGGCAAAGGAT
CACGTGGTTGAGGTGGAGCATGAATTCTACGTCAAAAAGGGTGGAGCCAAGAAAAGACCCGCCCCCAGTGACGCA
GATATAAGTGAGCCCAAACGGGTGCGCGAGTCAGTTGCGCAGCCATCGACGTCAGACGCGGAAGCTTCGATCAAC
TACGCAGACAGGTACCAAAACAAATGTTCTCGTCACGTGGGCATGAATCTGATGCTGTTTCCCTGCAGACAATGC
GAGAGAATGAATCAGAATTCAAATATCTGCTTCACTCACGGACAGAAAGACTGTTTAGAGTGCTTTCCCGTGTCA
GAATCTCAACCCGTTTCTGTCGTCAAAAAGGCGTATCAGAAACTGTGCTACATTCATCATATCATGGGAAAGGTG
CCAGACGCTTGCACTGCCTGCGATCTGGTCAATGTGGATTTGGATGACTGCATCTTTGAACAATAA
GFP coding sequence
SEQ ID NO: 36
ATGGTGAGCAAGGGCGAGGAGCTGTTCACCGGGGTGGTGCCCATCCTGGTCGAGCTGGACGGCGACGTAAACGGC
CACAAGTTCAGCGTGTCCGGCGAGGGCGAGGGCGATGCCACCTACGGCAAGCTGACCCTGAAGTTCATTTGCACC
ACCGGCAAGCTGCCCGTGCCCTGGCCCACCCTCGTGACCACCCTGACCTACGGCGTGCAGTGCTTCAGCCGCTAC
CCCGACCACATGAAGCAGCACGACTTCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGGCGACACCCTGGTGAACCGCATCGAG
CTGAAGGGCATCGACTTCAAGGAGGACGGCAACATCCTGGGGCACAAGCTGGAGTACAACTACAACAGCCACAAC
GTCTATATCATGGCCGACAAGCAGAAGAACGGCATCAAGGTGAACTTCAAGATCCGCCACAACATCGAGGACGGC
AGCGTGCAGCTCGCCGACCACTACCAGCAGAACACCCCCATCGGCGACGGCCCCGTGCTGCTGCCCGACAACCAC
TACCTGAGCACCCAGTCCGCCCTGAGCAAAGACCCCAACGAGAAGCGCGATCACATGGTCCTGCTGGAGTTCGTG
ACCGCCGCCGGGATCACTCTCGGCATGGACGAGCTGTACAAGTAA
the transgene plasmid comprising CoSMN2
SEQ ID NO: 37
TTAATTAACAATTGACATTATTGAAGCATTTATCAGGGTTATTGTCTCAGACTGCGCGCTCGCTCGCTCACTGAG
GCCGCCCGGGCAAAGCCCGGGCGTCGGGCGACCTTTGGTCGCCCGGCCTCAGTGAGCGAGCGAGCGCGCAGAGAG
GGAGTGTGTAGTTAATGATTAACCCGCCATGCTACTTATCTACCAGGGTAATGGGGATCCTCTAGAACTATAGCT
AGTCGACATAACGCGTCAATTGCTAGTCGACATTGATTATTGACTAGTTATTAATAGTAATCAATTACGGGGTCA
TTAGTTCATAGCCCATATATGGAGTTCCGCGTTACATAACTTACGGTAAATGGCCCGCCTGGCTGACCGCCCAAC
GACCCCCGCCCATTGACGTCAATAATGACGTATGTTCCCATAGTAACGCCAATAGGGACTTTCCATTGACGTCAA
TGGGTGGAGTATTTACGGTAAACTGCCCACTTGGCAGTACATCAAGTGTATCATATGCCAAGTACGCCCCCTATT
GACGTCAATGACGGTAAATGGCCCGCCTGGCATTATGCCCAGTACATGACCTTATGGGACTTTCCTACTTGGCAG
TACATCTACGTATTAGTCATCGCTATTACCATGGTCGAGGTGAGCCCCACGTTCTGCTTCACTCTCCCCATCTCC
CCCCCCTCCCCACCCCCAATTTTGTATTTATTTATTTTTTAATTATTTTGTGCAGCGATGGGGGGGGGGGGGGGG
GGGGGGCGCGCGCCAGGCGGGGCGGGGGGGGGCGAGGGGGGGGGGGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATAAAAAGCGAAGC
GCGCGGCGGGCGGAAGCTTGTAAGTATCAAGGTTACAAGACAGGTTTAAGGAGACCAATAGAAACTGGGCTTGTC
GAGACAGAGAAGACTCTTGCGTTTCTGATAGGCACCTATTGGTCTTACTGACATCCACTTTGCCTTTCTCTCCAC
AGGGCCTCGAGGACGGGGTGAACTACGCCTGAGGATCCGCCACCATGGCGATGAGCAGCGGCGGCAGTGGTGGCG
GCGTCCCGGAGCAGGAGGATTCCGTGCTGTTCCGGCGCGGCACAGGCCAGAGCGATGATTCTGACATTTGGGATG
ATACAGCACTGATAAAAGCATATGATAAAGCTGTGGCTTCATTTAAGCATGCTCTAAAGAATGGTGACATTTGTG
AAACTTCGGGTAAACCAAAAACCACACCTAAAAGAAAACCTGCTAAGAAGAATAAAAGCCAAAAGAAGAATACTG
CAGCTTCCTTACAACAGTGGAAAGTTGGGGACAAATGTTCTGCCATTTGGTCAGAAGACGGTTGCATTTACCCAG
CTACCATTGCTTCAATTGATTTTAAGAGAGAAACCTGTGTTGTGGTTTACACTGGATATGGAAATAGAGAGGAGC
AAAATCTGTCCGATCTACTTTCCCCAATCTGTGAAGTAGCTAATAATATAGAACAAAATGCTCAAGAGAATGAAA
ATGAAAGCCAAGTTTCAACAGATGAAAGTGAGAACTCCAGGTCTCCTGGAAATAAATCAGATAACATCAAGCCCA
AATCTGCTCCATGGAACTCTTTTCTCCCTCCACCACCCCCCATGCCAGGGCCAAGACTGGGACCAGGAAAGCCAG
GTCTAAAATTCAATGGCCCACCACCGCCACCGCCACCACCACCACCCCACTTACTATCATGCTGGCTGCCTCCAT
TTCCTTCTGGACCACCAATAATTCCCCCACCACCTCCCATATGTCCAGATTCTCTTGATGATGCTGATGCTTTGG
GAAGTATGTTAATTTCATGGTACATGAGTGGCTATCATACTGGCTATTATATGGGTTTCAGACAAAATCAAAAAG
AAGGAAGGTGCTCACATTCCTTAAATTAATGTACAGGTACCTCGATGGATTTCCAAGGTTAATTCATTGGAATTG
AAAATTAACAGAGATCTAGAGCTGAATTCCTGCAGCCAGGGGGATCAGCCTGATCTTTTTCCCTCTGCCAAAAAT
TATGGGGACATCATGAAGCCCCTTGAGCATCTGACTTCTGGCTAATAAAGGAAATTTATTTTCATTGCAATAGTG
TGTTGGAATTTTTTGTGTCTCTCACTCGCGGACCGAGCGGCCGCGCCTAGGTAGATAAGTAGCATGGCGGGTTAA
TCATTAACTACAAGGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCGAGCGCGCAGCCCTGCCT
GCAGGCATTTAATTAACCTAATTCGGCAGCTTGAAGGAAATACTAAGGCAAAGGTACTGCAAGTGCTCGCAACAT
TCGCTTATGCGGATTATTGCCGTAGTGCCGCGACGCCGGGGGCAAGATGCAGAGATTGCCATGGTACAGGCCGTG
CGGTTGATATTGCCAAAACAGAGCTGTGGGGGAGAGTTGTCGAGAAAGAGTGCGGAAGATGCAAAGGCGTCGGCT
ATTCAAGGATGCCAGCAAGCGCAGCATATCGCGCTGTGACGATGCTAATCCCAAACCTTACCCAACCCACCTGGT
CACGCACTGTTAAGCCGCTGTATGACGCTCTGGTGGTGCAATGCCACAAAGAAGAGTCAATCGCAGACAACATTT
TGAATGCGGTCACACGTTAGCAGCATGATTGCCACGGATGGCAACATATTAACGGCATGATATTGACTTATTGAA
TAAAATTGGGTAAATTTGACTCAACGATGGGTTAATTCGCTCGTTGTGGTAGTGAGATGAAAAGAGGCGGCGCTT
ACTACCGATTCCGCCTAGTTGGTCACTTCGACGTATCGTCTGGAACTCCAACCATCGCAGGCAGAGAGGTCTGCA
AAATGCAATCCCGAAACAGTTCGCAGGTAATAGTTAGAGCCTGCATAACGGTTTCGGGATTTTTTATATCTGCAC
AACAGGTAAGAGCATTGAGTCGATAATCGTGAAGAGTCGGCGAGCCTGGTTAGCCAGTGCTCTTTCCGTTGTGCT
GAATTAAGCGAATACCGGAAGCAGAACCGGATCACCAAATGCGTACAGGCGTCATCGCCGCCCAGCAACAGCACA
ACCCAAACTGAGCCGTAGCCACTGTCTGTCCTGAATTCATTAGTAATAGTTACGCTGCGGCCTTTTACACATGAC
CTTCGTGAAAGCGGGTGGCAGGAGGTCGCGCTAACAACCTCCTGCCGTTTTGCCCGTGCATATCGGTCACGAACA
AATCTGATTACTAAACACAGTAGCCTGGATTTGTTCTATCAGTAATCGACCTTATTCCTAATTAAATAGAGCAAA
TCCCCTTATTGGGGGTAAGACATGAAGATGCCAGAAAAACATGACCTGTTGGCCGCCATTCTCGCGGCAAAGGAA
CAAGGCATCGGGGCAATCCTTGCGTTTGCAATGGCGTACCTTCGCGGCAGATATAATGGCGGTGCGTTTACAAAA
ACAGTAATCGACGCAACGATGTGCGCCATTATCGCCTGGTTCATTCGTGACCTTCTCGACTTCGCCGGACTAAGT
AGCAATCTCGCTTATATAACGAGCGTGTTTATCGGCTACATCGGTACTGACTCGATTGGTTCGCTTATCAAACGC
TTCGCTGCTAAAAAAGCCGGAGTAGAAGATGGTAGAAATCAATAATCAACGTAAGGCGTTCCTCGATATGCTGGC
GTGGTCGGAGGGAACTGATAACGGACGTCAGAAAACCAGAAATCATGGTTATGACGTCATTGTAGGCGGAGAGCT
ATTTACTGATTACTCCGATCACCCTCGCAAACTTGTCACGCTAAACCCAAAACTCAAATCAACAGGCGCCGGACG
CTACCAGCTTCTTTCCCGTTGGTGGGATGCCTACCGCAAGCAGCTTGGCCTGAAAGACTTCTCTCCGAAAAGTCA
GGACGCTGTGGCATTGCAGCAGATTAAGGAGCGTGGCGCTTTACCTATGATTGATCGTGGTGATATCCGTCAGGC
AATCGACCGTTGCAGCAATATCTGGGCTTCACTGCCGGGCGCTGGTTATGGTCAGTTCGAGCATAAGGCTGACAG
CCTGATTGCAAAATTCAAAGAAGCGGGCGGAACGGTCAGAGAGATTGATGTATGAGCAGAGTCACCGCGATTATC
TCCGCTCTGGTTATCTGCATCATCGTCTGCCTGTCATGGGCTGTTAATCATTACCGTGATAACGCCATTACCTAC
AAAGCCCAGCGCGACAAAAATGCCAGAGAACTGAAGCTGGCGAACGCGGCAATTACTGACATGCAGATGCGTCAG
CGTGATGTTGCTGCGCTCGATGCAAAATACACGAAGGAGTTAGCTGATGCTAAAGCTGAAAATGATGCTCTGCGT
GATGATGTTGCCGCTGGTCGTCGTCGGTTGCACATCAAAGCAGTCTGTCAGTCAGTGCGTGAAGCCACCACCGCC
TCCGGCGTGGATAATGCAGCCTCCCCCCGACTGGCAGACACCGCTGAACGGGATTATTTCACCCTCAGAGAGAGG
CTGATCACTATGCAAAAACAACTGGAAGGAACCCAGAAGTATATTAATGAGCAGTGCAGATAGAGTTGCCCATAT
CGATGGGCAACTCATGCAATTATTGTGAGCAATACACACGCGCTTCCAGCGGAGTATAAATGCCTAAAGTAATAA
AACCGAGCAATCCATTTACGAATGTTTGCTGGGTTTCTGTTTTAACAACATTTTCTGCGCCGCCACAAATTTTGG
CTGCATCGACAGTTTTCTTCTGCCCAATTCCAGAAACGAAGAAATGATGGGTGATGGTTTCCTTTGGTGCTACTG
CTGCCGGTTTGTTTTGAACAGTAAACGTCTGTTGAGCACATCCTGTAATAAGCAGGGCCAGCGCAGTAGCGAGTA
GCATTTTTTTCATGGTGTTATTCCCGATGCTTTTTGAAGTTCGCAGAATCGTATGTGTAGAAAATTAAACAAACC
CTAAACAATGAGTTGAAATTTCATATTGTTAATATTTATTAATGTATGTCAGGTGCGATGAATCGTCATTGTATT
CCCGGATTAACTATGTCCACAGCCCTGACGGGGAACTTCTCTGCGGGAGTGTCCGGGAATAATTAAAACGATGCA
CACAGGGTTTAGCGCGTACACGTATTGCATTATGCCAACGCCCCGGTGCTGACACGGAAGAAACCGGACGTTATG
ATTTAGCGTGGAAAGATTTGTGTAGTGTTCTGAATGCTCTCAGTAAATAGTAATGAATTATCAAAGGTATAGTAA
TATCTTTTATGTTCATGGATATTTGTAACCCATCGGAAAACTCCTGCTTTAGCAAGATTTTCCCTGTATTGCTGA
AATGTGATTTCTCTTGATTTCAACCTATCATAGGACGTTTCTATAAGATGCGTGTTTCTTGAGAATTTAACATTT
ACAACCTTTTTAAGTCCTTTTATTAACACGGTGTTATCGTTTTCTAACACGATGTGAATATTATCTGTGGCTAGA
TAGTAAATATAATGTGAGACGTTGTGACGTTTTAGTTCAGAATAAAACAATTCACAGTCTAAATCTTTTCGCACT
TGATCGAATATTTCTTTAAAAATGGCAACCTGAGCCATTGGTAAAACCTTCCATGTGATACGAGGGCGCGTAGTT
TGCATTATCGTTTTTATCGTTTCAATCTGGTCTGACCTCCTTGTGTTTTGTTGATGATTTATGTCAAATATTAGG
AATGTTTTCACTTAATAGTATTGGTTGCGTAACAAAGTGCGGTCCTGCTGGCATTCTGGAGGGAAATACAACCGA
CAGATGTATGTAAGGCCAACGTGCTCAAATCTTCATACAGAAAGATTTGAAGTAATATTTTAACCGCTAGATGAA
GAGCAAGCGCATGGAGCGACAAAATGAATAAAGAACAATCTGCTGATGATCCCTCCGTGGATCTGATTCGTGTAA
AAAATATGCTTAATAGCACCATTTCTATGAGTTACCCTGATGTTGTAATTGCATGTATAGAACATAAGGTGTCTC
TGGAAGCATTCAGAGCAATTGAGGCAGCGTTGGTGAAGCACGATAATAATATGAAGGATTATTCCCTGGTGGTTG
ACTGATCACCATAACTGCTAATCATTCAAACTATTTAGTCTGTGACAGAGCCAACACGCAGTCTGTCACTGTCAG
GAAAGTGGTAAAACTGCAACTCAATTACTGCAATGCCCTCGTAATTAAGTGAATTTACAATATCGTCCTGTTCGG
AGGGAAGAACGCGGGATGTTCATTCTTCATCACTTTTAATTGATGTATATGCTCTCTTTTCTGACGTTAGTCTCC
GACGGCAGGCTTCAATGACCCAGGCTGAGAAATTCCCGGACCCTTTTTGCTCAAGAGCGATGTTAATTTGTTCAA
TCATTTGGTTAGGAAAGCGGATGTTGCGGGTTGTTGTTCTGCGGGTTCTGTTCTTCGTTGACATGAGGTTGCCCC
GTATTCAGTGTCGCTGATTTGTATTGTCTGAAGTTGTTTTTACGTTAAGTTGATGCAGATCAATTAATACGATAC
CTGCGTCATAATTGATTATTTGACGTGGTTTGATGGCCTCCACGCACGTTGTGATATGTAGATGATAATCATTAT
CACTTTACGGGTCCTTTCCGGTGATCCGACAGGTTACGGCCTGATGCGGTATTTTCTCCTTACGCATCTGTGCGG
TATTTCACACCGCATACGTCAAAGCAACCATAGTACGCGCCCTGTAGCGGCGCATTAAGCGCGGCGGGTGTGGTG
GTTACGCGCAGCGTGACCGCTACACTTGCCAGCGCCCTAGCGCCCGCTCCTTTCGCTTTCTTCCCTTCCTTTCTC
GCCACGTTCGCCGGCTTTCCCCGTCAAGCTCTAAATCGGGGGCTCCCTTTAGGGTTCCGATTTAGTGCTTTACGG
CACCTCGACCCCAAAAAACTTGATTTGGGTGATGGTTCACGTAGTGGGCCATCGCCCTGATAGACGGTTTTTCGC
CCTTTGACGTTGGAGTCCACGTTCTTTAATAGTGGACTCTTGTTCCAAACTGGAACAACACTCAACCCTATCTCG
GGCTATTCTTTTGATTTATAAGGGATTTTGCCGATTTCGGCCTATTGGTTAAAAAATGAGCTGATTTAACAAAAA
TTTAACGCGAATTTTAACAAAATATTAACGTTTACAATTTTATGGTGCACTCTCAGTACAATCTGCTCTGATGCC
GCATAGTTAAGCCAGCCCCGACACCCGCCAACACCCGCTGACGCGCCCTGACGGGCTTGTCTGCTCCCGGCATCC
GCTTACAGACAAGCTGTGACCGTCTCCGGGAGCTGCATGTGTCAGAGGTTTTCACCGTCATCACCGAAACGCGCG
AGACGAAAGGGCCTCGTGATACGCCTATTTTTATAGGTTAATGTCATGATAATAATGGTTTCTTAGACGTCAGGT
GGCACTTTTCGGGGAAATGTGCGCGGAACCCCTATTTGTTTATTTTTCTAAATACATTCAAATATGTATCCGCTC
ATGAGACAATAACCCTGATAAATGCTTCAATAATATTGAAAAAGGAAGAGTATGAGCCATATTCAACGGGAAACG
TCGAGGCCGCGATTAAATTCCAACATGGATGCTGATTTATATGGGTATAAATGGGCTCGCGATAATGTCGGGCAA
TCAGGTGCGACAATCTATCGCTTGTATGGGAAGCCCGATGCGCCAGAGTIGTTTCTGAAACATGGCAAAGGTAGC
GTTGCCAATGATGTTACAGATGAGATGGTCAGACTAAACTGGCTGACGGAATTTATGCCTCTTCCGACCATCAAG
CATTTTATCCGTACTCCTGATGATGCATGGTTACTCACCACTGCGATCCCCGGAAAAACAGCATTCCAGGTATTA
GAAGAATATCCTGATTCAGGTGAAAATATTGTTGATGCGCTGGCAGTGTTCCTGCGCCGGTTGCATTCGATTCCT
GTTTGTAATTGTCCTTTTAACAGCGATCGCGTATTTCGTCTCGCTCAGGCGCAATCACGAATGAATAACGGTTTG
GTTGATGCGAGTGATTTTGATGACGAGCGTAATGGCTGGCCTGTTGAACAAGTCTGGAAAGAAATGCATAAACTT
TTGCCATTCTCACCGGATTCAGTCGTCACTCATGGTGATTTCTCACTTGATAACCTTATTTTTGACGAGGGGAAA
TTAATAGGTTGTATTGATGTTGGACGAGTCGGAATCGCAGACCGATACCAGGATCTTGCCATCCTATGGAACTGC
CTCGGTGAGTTTTCTCCTTCATTACAGAAACGGCTTTTTCAAAAATATGGTATTGATAATCCTGATATGAATAAA
TTGCAGTTTCATTTGATGCTCGATGAGTTTTTCTAACTGTCAGACCAAGTTTACTCATATATACTTTAGATTGAT
TTAAAACTTCATTITTAATTTAAAAGGATCTAGGTGAAGATCCTTTTTGATAATCTCATGACCAAAATCCCTTAA
CGTGAGTTTTCGTTCCACTGAGCGTCAGACCCCGTAGAAAAGATCAAAGGATCTTCTTGAGATCCTTTTTTTCTG
CGCGTAATCTGCTGCTTGCAAACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTTCTTCTAGTGTAGCCGTAGTTA
GGCCACCACTTCAAGAACTCTGTAGCACCGCCTACATACCTCGCTCTGCTAATCCTGTTACCAGTGGCTGCTGCC
AGTGGCGATAAGTCGTGTCTTACCGGGTTGGACTCAAGACGATAGTTACCGGATAAGGCGCAGCGGTCGGGCTGA
ACGGGGGGTTCGTGCACACAGCCCAGCTTGGAGCGAACGACCTACACCGAACTGAGATACCTACAGCGTGAGCTA
TGAGAAAGCGCCACGCTTCCCGAAGGGAGAAAGGCGGACAGGTATCCGGTAAGCGGCAGGGTCGGAACAGGAGAG
CGCACGAGGGAGCTTCCAGGGGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCAGCAACGCGGCCTTTTTACGGTTC
CTGGCCTTTTGCTGGCCTTTTGCTCACATGT
Forward primer for ddPCR
SEQ ID NO: 38
CCCACTTGGCAGTACATCAA
Reverse primer for ddPCR
SEQ ID NO: 39
GCCAAGTAGGAAAGTCCCATAA
Probe for ddPCR
SEQ ID NO: 40
CATAATGCCAGGCGGGCCATTTAC
Forward primer for qPCR of mouse GAPDH
SEQ ID NO: 41
CCACTCACGGCAAATTCAAC
Reverse primer for qPCR of mouse GAPDH
SEQ ID NO: 42
ACCAGTAGACTCCACGACATA
Probe for qPCR of mouse GAPDH
SEQ ID NO: 43
TTGTCATCAACGGGAAGCCCATCA
Forward primer for qPCR of CoSMN11
SEQ ID NO: 44
CTATCATCCCTCCTCCTCCTC
Reverse primer for qPCR of CoSMN11
SEQ ID NO: 45
GCCTGAAGCCCATGTAGTAG
Probe for qPCR of CoSMN11
SEQ ID NO: 46
CTGGTACATGAGCGGCTACCACAC

1. A recombinant adeno-associated virus (rAAV) comprising a genome comprising an expression construct comprising a polynucleotide of interest that comprises a nucleotide sequence selected from SEQ ID NOs: 2, 3, 4, 5, 6, 7, 8, 9, 10, 11, 12, 13, 14, 15, 16, 17, 18, 19 and 20, and is operably linked to a promoter.

2. The rAAV of claim 1, wherein the construct further comprises an intron.

3. The rAAV of claim 2, wherein the intron is between the promoter and the polynucleotide of interest.

4. The rAAV of claim 2, wherein the intron comprises a nucleotide sequence selected from the group consisting of SEQ ID NOs: 24 and 29.

5. The rAAV of claim 1, wherein the construct further comprises an enhancer.

6. The rAAV of claim 5, wherein the enhancer is upstream of the promoter.

7. The rAAV of claim 5, wherein the enhancer comprises a nucleotide sequence of SEQ ID NO: 22.

8. The rAAV of claim 1, wherein the promoter comprises a nucleotide sequence selected from a group consisting of SEQ ID NOs: 23, 28 and 31.

9. A pharmaceutical composition comprising the rAAV of claim 1.

10. The rAAV of claim 1, for use in the treatment of a disease associated with the deficiency of SMN1 gene.

11. The rAAV or the pharmaceutical composition of claim 10, wherein the disease is spinal muscular atrophy (SMA).
